(19) United States
(12) Patent Application Publication
(10) Pub. No.: US 2026/0345778 A1
(43) Pub. Date: Nov. 5, 2026

(54) REDIRECTING GLUCOSE METABOLISM TO LIMIT STRESS AND IMPROVE ADOPTIVE CELL THERAPY

(71) Applicant: University of Pittsburgh - Of the Commonwealth System of Higher Education, Pittsburgh, PA (US)

(72) Inventors: Greg M. Delgoffe, Pittsburgh, PA (US); Andrew Tyler Frisch, Pittsburgh, PA (US)

(73) Assignee: University of Pittsburgh - Of the Commonwealth System of Higher Education, Pittsburgh, PA (US)

(21) Appl. No.: 19/738,197

(22) Filed: Jul. 10, 2026

Related U.S. Application Data
(62) Division of application No. 18/034,027, filed on Oct. 27, 2021.
(60) Provisional application No. 63/106,340, filed on Oct. 27, 2020.

(51) Int. Cl. A61K 40/31; A61K 40/11; A61K 40/32; A61K 40/42; A61P 35/00; C12N 5/0783

(52) U.S. Cl. CPC A61K 40/31; A61K 40/11; A61K 40/32; A61K 40/4204; A61K 40/4211; A61K 40/4271; A61P 35/00; C12N 5/0636; A61K 2239/57; C12N 2501/2302; C12N 2501/515; C12N 2501/599; C12N 2501/727; C12N 2510/00

(57) ABSTRACT

Provided are improved methods for making immune effector cells, as well as improved immune effector cells generated using the methods. The improved T cells have improved respiratory capacity and mitochondrial mass. Adoptive T cell immunotherapies using such cells demonstrate improved survival, expansion, and persistence in vivo.

### CROSS REFERENCE TO RELATED APPLICATIONS

This application is a divisional of U.S. patent application Ser. No. 18/034,027, filed Apr. 26, 2023, which is the U.S. National Stage of International Application No. PCT/US2021/056898, filed Oct. 27, 2021, which in turn claims priority to U.S. Provisional Application No. 63/106,340 filed Oct. 27, 2020, all of which are incorporated by reference herein in their entireties.

### FIELD

The present disclosure relates to improved methods for manufacturing immune effector cells, as well as improved immune effector cells generated using the methods, and methods of using the immune effector cells. The new methods of immune effector cell manufacturing result in immune effector cells with improved metabolic profiles, respiratory capacity, and/or mitochondrial mass and adoptive T cell immunotherapies with improved survival, expansion, and persistence in vivo.

### INCORPORATION OF SEQUENCE LISTING

The Sequence Listing is submitted as an xml text file named “Sequence.xml” which was created on Jul. 10, 2026, and is 42,415 bytes, which is incorporated by reference herein.

### BACKGROUND

Adoptive immunotherapy is the transfer of T lymphocytes to a subject to provide therapy for a disease. Adoptive immunotherapy has yet unrealized potential for treating a wide variety of diseases including cancer, infectious disease, autoimmune disease, inflammatory disease, and immunodeficiency. However, most, if not all adoptive immunotherapy strategies require T cell activation and expansion steps to generate a clinically effective, therapeutic dose of T cells. Current technologies for generating therapeutic doses of T cells, including engineered T cells, remain limited by cumbersome T cell manufacturing processes. For example, T cell expansion often requires labor intensive and expensive cloning, and/or multiple rounds of activation/expansion to achieve therapeutically relevant T cell numbers. In addition, existing T cell activation/expansion methods are normally coupled with substantial T cell differentiation and usually result in short-lived effects, including short-lived survival and a lack of persistence and lack of in vivo expansion of the transferred T cells. Thus, existing T cell manufacturing processes produce an inferior T cell product that is prone to exhaustion and loss of effector immune cell function.

To date, clinical efficacy of engineered T cell adoptive immunotherapies is limited by poor T cell expansion and persistence after infusion into patients. Therefore, such therapies are not suitable for widespread clinical use. Accordingly, there is a persistent, unmet need for improvements in T cell manufacturing and therapeutic T cell compositions that survive, expand, and persist in vivo.

### SUMMARY

Provided herein are adoptive T cell immunotherapies comprising persistent and potent anti-tumor T cell compositions and methods of making the same. Also provided are improved T cell compositions and methods for manufacturing T cells. More particularly, the methods of T cell manufacturing result in improved metabolic profiles, respiratory capacity, and/or mitochondrial mass of the T cells, thereby increasing their survival, expansion, and/or persistence in vivo. Without being bound by any particular theory, it is proposed that culturing immune effector cells (such as T cells) that have been activated, stimulated, and/or transduced, in the presence of one or more metabolic enhancers selected from the group consisting of pyruvate dehydrogenase kinase 1 (PDHK1) inhibitors, pyruvate dehydrogenase phosphatase (PDP) activators, peroxisome proliferator-activated receptor gamma coactivator 1-alpha (PGC1α) polypeptides or variants thereof, and PGC1α agonists, redirects glucose to the mitochondria away from fermentation into lactate (e.g., towards oxidation, antagonizes glycolysis promoting signature during expansion/culturing step) (see, FIG. 1). In some examples, such a method increases the respiratory capacity of the transduced immune effector cells by at least 10%, at least 20%, at least 30%, at least 40%, or at least 50%, relative to transduced immune effector cells (e.g., T cells) cultured in the absence of the one or more of the metabolic enhancers. This is in contrast to prior methods that proposed inhibiting glycolysis at the activation and stimulation step.

Provided herein are methods for manufacturing immune effector cells. Such methods can include (a) activating a population of immune effector cells; (b) transducing the immune effector cells with a vector comprising a polynucleotide encoding an engineered antigen receptor; and (c) culturing the transduced immune effector cells to proliferate; wherein any one or more of steps (a)-(c) is performed in the presence of one or more metabolic enhancers selected from the group consisting of pyruvate dehydrogenase kinase 1 (PDHK1) inhibitors, pyruvate dehydrogenase phosphatase (PDP) activators, peroxisome proliferator-activated receptor gamma coactivator 1-alpha (PGC1α) polypeptides or variants thereof, and PGC1α agonists. Such a method can increase the respiratory capacity of the transduced immune effector cells by at least 10% relative to transduced immune effector cells cultured in the absence of the one or more of the metabolic enhancers.

Provided herein are methods for increasing respiratory capacity of immune effector cells. Such methods can include (a) activating a population of immune effector cells; (b) transducing the immune effector cells with a vector comprising a polynucleotide encoding an engineered antigen receptor; and (c) culturing the transduced immune effector cells to proliferate, wherein any one or more of steps (a)-(c) is performed in the presence of one or more metabolic enhancers selected from the group consisting of PDHK1 inhibitors, PDP activators, PGC1α polypeptides or variants thereof, and PGC1α agonists. Such a method can increase the respiratory capacity of the transduced immune effector cells is increased by at least 10% relative to transduced immune effector cells cultured in the absence of the one or more of the metabolic enhancers.

Provided herein are methods for increasing the mitochondrial mass of immune effector cells. Such methods can include (a) activating a population of immune effector cells; (b) transducing the immune effector cells with a vector comprising a polynucleotide encoding an engineered antigen receptor; and (c) culturing the transduced immune effector cells to proliferate; wherein any one or more of steps (a)-(c) is performed in the presence of one or more metabolic enhancers selected from the group consisting of PDHK1 inhibitors, PDP activators, PGC1α polypeptides or variants thereof, and PGC1α agonists. Such a method can increase the mitochondrial mass of the transduced immune effector cells by at least 25%, at least 30%, at least 40%, or at least 50%, relative to transduced immune effector cells cultured in the absence of the one or more of the metabolic enhancers.

Also provided is a population of immune effector cells generated using the disclosed methods. Such a population of immune effector cells can in some examples include a vector comprising an engineered antigen receptor (such as a TCR, DARIC, or CAR), and thus have increased respiratory capacity and/or mitochondrial mass as described herein. In certain embodiments, the immune effector cells are TILs. In various embodiments, a composition comprising a population of immune effector cells comprising a vector comprising an engineered antigen receptor (such as a TCR, DARIC, or CAR), produced using the culturing methods provided herein is provided. Such a composition can further include a physiologically acceptable excipient. Also provided are kits that include a population of immune effector cells comprising a vector comprising an engineered antigen receptor (such as a TCR, DARIC, or CAR), produced using the culturing methods provided herein, optionally further including one or more materials to allow administration of the immune effector cells, and/or additional anti-cancer agents.

In various certain embodiments, methods of treating a cancer, infectious disease, autoimmune disease, inflammatory disease, or immunodeficiency in a subject in need thereof, which include administering to the subject a therapeutically effect amount of an immune effector cell composition provided herein, are provided. In particular embodiments, the cancer is selected from the group consisting of Wilms' tumor, Ewing sarcoma, a neuroendocrine tumor, a glioblastoma, a neuroblastoma, a melanoma, skin cancer, breast cancer, colon cancer, rectal cancer, prostate cancer, liver cancer, renal cancer, pancreatic cancer, lung cancer, biliary cancer, cervical cancer, endometrial cancer, esophageal cancer, gastric cancer, head and neck cancer, medullary thyroid carcinoma, ovarian cancer, glioma, lymphoma, leukemia, myeloma, acute lymphoblastic leukemia, acute myelogenous leukemia, chronic lymphocytic leukemia, chronic myelogenous leukemia, Hodgkin's lymphoma, non-Hodgkin's lymphoma, and urinary bladder cancer.

The foregoing and other objects and features of the disclosure will become more apparent from the following detailed description, which proceeds with reference to the accompanying figures.

### BRIEF DESCRIPTION OF THE DRAWINGS

FIG. 1 shows a schematic representation of the effects of inhibition of PDHK1 (for example with DCA). By inhibiting pyruvate dehydrogenase kinase, DCA redirects the flow of glucose-derived pyruvate into the mitochondria for oxidation and away from the fermentation pathways typically stimulated in the presence of TCR, CD28, and IL2.

FIG. 2 shows a schematic summarizing various means for improving T cell therapies. These approaches include, but are not limited to, the enhancement of T cell longevity, improvement in mitochondrial health, antagonizing glycolysis promoting signals during expansion, use of various PDHK1 inhibitors, transient knockdown of PDHK1, combined use of DCA and other metabolic modulators during expansion, transient overexpression of PDP, and the use of particular CAR-T cells for specific types of treatment.

FIG. 3 shows improved survival in T cells expanded in DCA as measured by expression of anti-apoptotic BLC2.

FIGS. 4A-4E show improved mitochondrial respiratory capacity in an immunocompetent solid tumor mouse model for T cells expanded in DCA as determined by oxygen consumption rate (OCR) and extracellular acidification rate (ECAR). (A) is a graph of OCR versus time illustrating the effect on metabolism of the addition of various metabolic modulators, as discussed in Example 2. (B) is a bar graph showing a comparison of OCR under different parallel conditions for a T cell group expanded in 5 mM DCA versus a control group. (C) is a bar graph showing a comparison of ECAR under different parallel conditions for a T cell group expanded in 5 mM DCA versus a control group. (D) is an experimental repeat of (A). (E) shows mitochondrial mass as measured by MitoTracker®FM.

FIG. 5A shows a schematic of the in vivo experimental protocol discussed in Example 3, including B16 tumor growth in mice in combination with the adoptive transfer of pmel T cells expanded in 5 mM DCA. As shown in FIGS. 5B-C, pmel T cells expanded in DCA result in a superior metabolic phenotype, for example by treating the tumor more effectively and increasing the survival time of the mice, as compared to T cells not treated with DCA. FIG. 5D (left) shows that adoptive transfer of DCA-treated pmel T cells results in increased cytokine production, as assessed by increased intra-tumoral IFN-gamma proliferation. FIG. 5D (right) shows that adoptive transfer of DCA-treated pmel T cells results in increased T cell proliferation and reduced apoptosis, as assessed by Ki67 expression. FIGS. SE-F show an experimental repeat of FIGS. 5B-C.

FIGS. 6A-6D depicts improved mitochondrial respiratory capacity in human T cells expanded in DCA as determined by oxygen consumption rate (OCR) and extracellular acidification rate (ECAR). FIG. 6A shows a schematic of the method. PBMCs are activated and stimulated in the presence of CD3 and CD28 for 48 hours, then cultured in PDHK1 inhibitor (DCA) for 10 days. FIG. 6B is a graph displaying OCR of the DCA treated and untreated PBMC over time, showing an increase in OCR for T cells expanded in 5 mM DCA versus control. FIG. 6C shows an increase of at least 20 pmol/min in OCR over baseline observed in T cells in expanded in 5 mM DCA versus control. FIG. 6D demonstrates a Spare Respiratory Capacity (“SRC”) of at least 60 (OCCRmax−OCRbasal) for T cells expanded in 5 mM DCA versus control.

FIG. 7A-7C show that DCA metabolically rescues tumor-infiltrating T cells when expanded in vitro in PBLs from PBMCs of cancer patients. FIG. 7A shows a schematic of this in vivo experimental protocol. FIG. 7B shows a metabolic flux rate experiment wherein OCR is evaluated at serial time points. DCA-treated T cells show a superior metabolic phenotype at 5 mM DCA and 10 mM DCA relative to TIL control. FIG. 7C shows that DCA-treated cells have a superior metabolic phenotype, including an enhanced immune response and decreased apoptosis. An enhanced immune response is shown by increased cytokine production, as assessed by enhanced intra-tumoral IFN-gamma proliferation and TNF-alpha proliferation. Enhanced proliferation and decreased apoptosis is shown by the increasing ratio of: PD-1 negative T cells to PD-1 positive T cells and, Tim-3 negative T cells to Tim-3 positive T cells.

FIG. 8A shows the results of a metabolic flux rate experiment, demonstrating an at least 10-fold enhancement in respiratory capacity achieved for mice with T cells expanded in 5 mM DCA. FIG. 8B shows that, in a metabolic flux rate experiment, DCA-treated human anti-CD19 CAR-T cells show a superior metabolic phenotype at 5 mM DCA relative to a hCD19 CAR control. FIG. 8C shows that the observations in FIGS. 8A-8B are associated with enhanced survival, and FIG. 8D shows tumor growth is reduced to a greater extent when T cells exposed to DCA are administered (as compared to administration of untreated (UTD) or T cells without exposure to DCA).

FIGS. 9A-9C depict metabolic flux rate experiments and flow cytometry measurements, showing that DCA can metabolically enhance a solid-tumor targeted CAR. FIG. 9A shows that DCA-treated EGFR BBz CAR-T cells display a superior metabolic phenotype at 5 mM DCA and 10 mM DCA relative to UTD control. FIG. 9B shows that DCA-treated EGFR BBz CAR-T cells display a superior metabolic phenotype at 5 mM DCA and 10 mM DCA relative to UTD control as measured by ECAR analysis. FIG. 9C measures flow cytometry via MitoTracker® Deep Red FM and competency for glucose uptake via fluorescent 2-NBD-glucose (2NBDG). These measurements show that the observations in FIGS. 9A-9B are associated with a superior effector phenotype, including reduced mitochondrial mass, improved mitochondrial function, and enhanced glucose uptake.

FIG. 10 depicts exemplary conditions for mouse T cell expansion with DCA. On the first day (“day 0”), 1) mouse T cells were seeded with CD3 and CD28 antibodies or whole mouse splenocytes, and 2) T cell stimulation cocktail is added. After 24 hours (“day 1”), 3) stimulation is removed and cells are fed, and 4) DCA is added, for example to a final concentration of 5 mM. On days 2-6, 5) cells are fed to a concentration of 5×10⁵ cells/mL adding 50 IU/mL IL-2 and 5 mM DCA to new media. On day 7, 6) endpoint analysis and in vivo transfer is performed. Standard R10 media culture includes 950 mL RPMI, 100 mL FBS, 10 mL Sodium Pyruvate, 10 mL NEAA, 5 mL HEPES, 1 mL BME, 10 mL and L-glutamine.

FIG. 11 depicts exemplary conditions for human CAR-T cell production with DCA. On the first day (“day 0”), 1) PBMCs are seeded in T Cell Growth Media (TCGM), and 2) T cell stimulation cocktail is added. After 24 hours (“day 1”), 3) viral transduction is carried out with CAR constructs, and 4) DCA is added, for example to a final concentration of 5 mM. On day 3, 5) viral supernatant is removed, and 6) cells are fed with new media. On day 4, 7) a transfer of cells is made, for example to G-Rex® 6 multi-well plates. On day 6, 8) DCA is added, for example to a final concentration of 5 mM. On day 10, 9) cells are frozen. Standard T Cell Growth Media includes: 930 mL X-VIVO®-15, 50 mL Human AB Serum, 10 mL 100× GlutaMAX®, and 10 mL 1M HEPES.

FIG. 12A is a schematic of an in vivo experimental protocol discussed in Example 9. FIG. 12B shows that DCA pre-treatment improves survival of rechallenged mice.

FIGS. 13A-13B show that DCA treatment improves T cell persistence. FIG. 13A shows % Live CD8+ Thy1.1+ cells circulating 20 or 60 days after second tumor implant, generated from the flow cytometry data in FIG. 13B.

FIG. 14A is a schematic of an in vivo experimental protocol discussed in Example 9. FIGS. 14B-14C show that DCA treatment increases T cell viability post-infusion. FIG. 14C shows % untreated or DCA treated donor CD8 T cells in blood, spleen, or tumor in vivo at 24 hours, generated from the flow cytometry data in FIG. 14B.

FIG. 15A is a schematic of the experimental protocol for FIGS. 15B and 15C. Cells were cultured in the presence or absence of DCA for 7 days, then switched to low (1 mM glucose+0 mM pyruvate) or high (11.2 mM glucose+10 mM pyruvate) nutrient conditions for 14 hours. 15B and 15C show that DCA expanded T cells are more proficient at synthesizing cytokines when restimulated after expansion, and are more resistant to stress in low nutrient conditions.

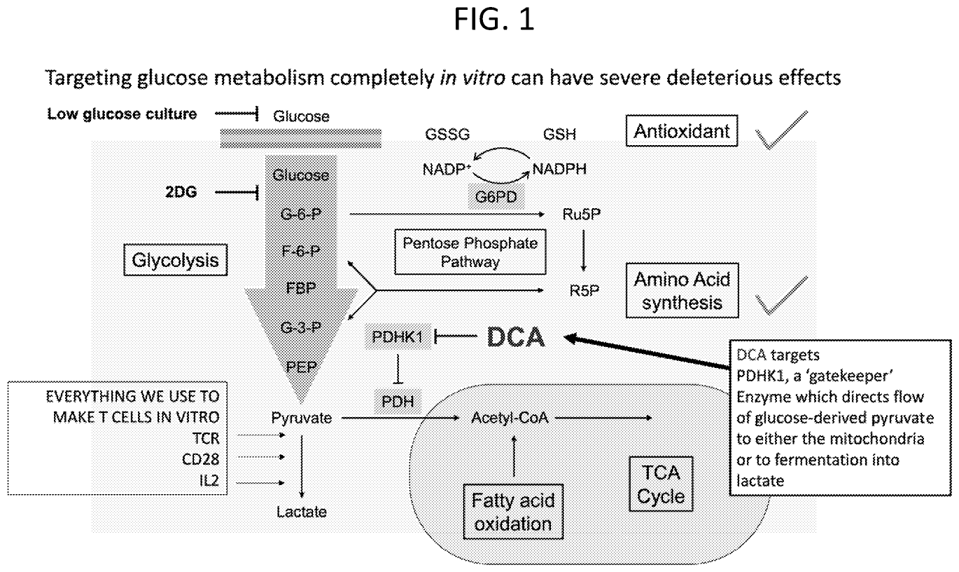

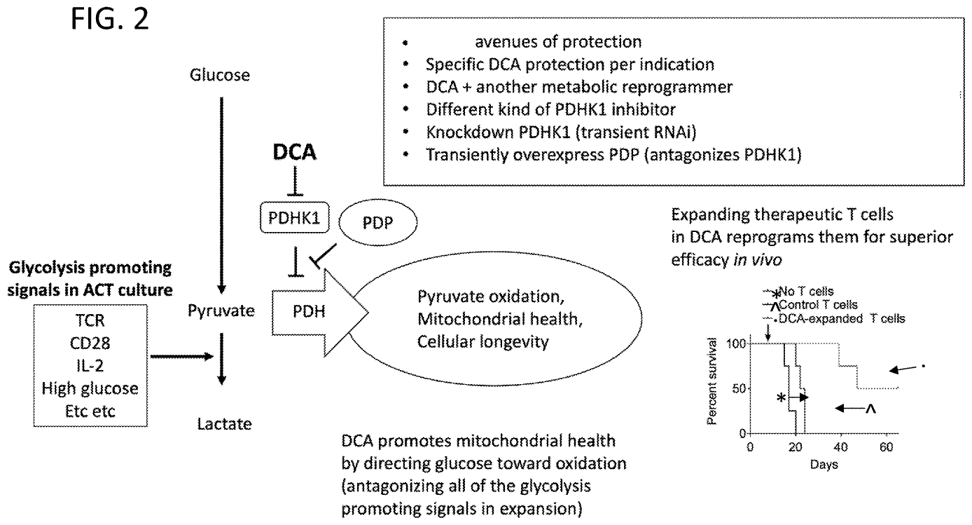

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. In the accompanying sequence listing:

SEQ ID NO: 1 is an exemplary coding sequence (Genbank® Accession No. BC039158.1) of wild-type human PDHK1, coding sequence nt 44-1354.

SEQ ID NO: 2 is an exemplary amino acid sequence (GenBank® Accession No. AAC42009.1) of wild-type human PDHK1.

SEQ ID NO: 3 is an exemplary coding sequence (NM_018444.4) of wild-type human PDP, coding sequence nt 202-1815.

SEQ ID NO: 4 is an exemplary amino acid sequence (NP_060914.2) of wild-type human PDP, isoform 1.

SEQ ID NOS: 5-7 are exemplary TEV (tobacco etch virus) protease cleavage sites).

SEQ ID NOS: 8-17 are exemplary linker sequences.

SEQ ID NO: 18 is an exemplary amino acid sequence (GenBank® Accession No. Q9UBK2.1) of wild-type human PGC-1α isoform 2.

SEQ ID NO: 19 is an exemplary amino acid sequence (GenBank® Accession No. Q9UBK2.1) of wild-type human PGC-1α isoform 1.

SEQ ID NOS: 20 and 21 are exemplary PDHK1 phosphorothioate antisense oligonucleotides.

SEQ ID NOS: 22-24 are exemplary human PDK1 RNAi sequences.

### DETAILED DESCRIPTION

Unless otherwise noted, technical terms are used according to conventional usage. Aspects of the disclosed methods employ, unless indicated specifically to the contrary, conventional methods of chemistry, biochemistry, organic chemistry, molecular biology, microbiology, recombinant DNA techniques, genetics, immunology, and cell biology, many of which are described below for the purpose of illustration. Such techniques are explained fully in the literature. See, e.g., Sambrook, et al., Molecular Cloning: A Laboratory Manual (3rd Edition, 2001); Sambrook, et al., Molecular Cloning: A Laboratory Manual (2nd Edition, 1989); Maniatis et al., Molecular Cloning: A Laboratory Manual (1982); Ausubel et al., Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (TRL Press, Oxford, 1985); Anand, Techniques for the Analysis of Complex Genomes, (Academic Press, New York, 1992); Transcription and Translation (B. Hames & S. Higgins, Eds., 1984); Perbal, A Practical Guide to Molecular Cloning (1984); Harlow and Lane, Antibodies, (Cold Spring Harbor Laboratory Press, Cold Spring Harbor, N.Y., 1998) Current Protocols in Immunology Q. E. Coligan, A. M. Kruisbeek, D. H. Margulies, E. M. Shevach and W. Strober, eds., 1991); Annual Review of Immunology; as well as monographs in journals such as Advances in Immunology.

As used herein, the singular forms “a,” “an,” and “the,” may refer to both the singular as well as plural, unless the context clearly indicates otherwise. As used herein, the term “comprises” can mean “includes.” Thus, “comprising an immune effector cell” can mean “including an immune effector cell” without excluding other elements. It is further to be understood that any and all base sizes given for nucleic acids are approximate, and are provided for descriptive purposes, unless otherwise indicated. Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting. All references, including patent applications and patents, and sequences associated with the GenBank® Accession Numbers listed (as of Oct. 27, 2020) are herein incorporated by reference.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment.

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%.

As used herein, the term “substantially” refers to a quantity, level, value, number, frequency, percentage, dimension, size, amount, weight or length that is 80%, 85%, 90%, 91%, 92%, 93%, 94%, 95%, 96%, 97%, 98%, 99% or higher of a reference quantity, level, value, number, frequency, percentage, dimension, size, amount, weight or length. In one embodiment, “substantially the same” refers to a quantity, level, value, number, frequency, percentage, dimension, size, amount, weight or length that produces an effect, e.g., a physiological effect, that is approximately the same as a reference quantity, level, value, number, frequency, percentage, dimension, size, amount, weight or length.

### I. TERMS

In order to facilitate review of the various embodiments of the disclosure, the following explanations of specific terms are provided:

Activation: The state of an immune effector cell (e.g., T cell) that has been sufficiently stimulated to induce detectable cellular proliferation. In particular embodiments, activation can also be associated with induced cytokine production, and detectable effector functions. The term “activated immune effector cells” refers to, among other things, immune effector cells that are proliferating. Signals generated through the TCR alone are insufficient for full activation of a T cell and one or more secondary or costimulatory signals are also required. Thus, immune effector cell activation comprises a primary stimulation signal through the TCR/CD3 complex and one or more secondary costimulatory signals. Costimulation can be evidenced by proliferation and/or cytokine production by immune effector cells that have received a primary activation signal, such as stimulation through the CD3/TCR complex or through CD2.

Administration: To provide or give a subject an agent, such as a disclosed immune effector cell (e.g., T cell) or other therapeutic agent (such as an anti-cancer agent), by any effective route. Exemplary routes of administration include, but are not limited to, injection (such as subcutaneous, subdermal, intramuscular, intradermal, intraperitoneal, intracerebroventricular, intraosseous, intratumoral, intraprostatic, and intravenous), transdermal, intranasal, oral, vaginal, rectal, and inhalation routes.

Allogeneic: With respect to cells, refers to cells of the same species that differ genetically to the cell in comparison.

Antibody (Ab): A polypeptide ligand comprising at least one variable region that recognizes and binds (such as specifically recognizes and specifically binds) an epitope of an antigen. Mammalian immunoglobulin molecules are composed of a heavy (H) chain and a light (L) chain, each of which has a variable region, termed the variable heavy (V_{H}) region and the variable light (V_{L}) region, respectively. Together, the V_{H} region and the V_{L} region are responsible for binding the antigen recognized by the antibody. There are five main heavy chain classes (or isotypes) of mammalian immunoglobulin, which determine the functional activity of an antibody molecule: IgM, IgD, IgG, IgA and IgE. Antibody isotypes not found in mammals include IgX, IgY, IgW and IgNAR. IgY is the primary antibody produced by birds and reptiles, and has some functionally similar to mammalian IgG and IgE. IgW and IgNAR antibodies are produced by cartilaginous fish, while IgX antibodies are found in amphibians.

Antibody variable regions contain “framework” regions and hypervariable regions, known as “complementarity determining regions” or “CDRs.” The CDRs are primarily responsible for binding to an epitope of an antigen. The framework regions of an antibody serve to position and align the CDRs in three-dimensional space. The amino acid sequence boundaries of a given CDR can be readily determined using any of a number of numbering schemes, including those described by Kabat et al. (Sequences of Proteins of Immunological Interest, U.S. Department of Health and Human Services, 1991; the “Kabat” numbering scheme), Chothia et al. (see Chothia and Lesk, J Mol Biol 196:901-917, 1987; Chothia et al., Nature 342:877, 1989; and Al-Lazikani et al., (JMB 273,927-948, 1997; the “Chothia” numbering scheme), and the ImMunoGeneTics (IMGT) database (see, Lefranc, Nucleic Acids Res 29:207-9, 2001; the “IMGT” numbering scheme). The Kabat and IMGT databases are maintained online.

A “single-domain antibody” refers to an antibody having a single domain (a variable domain) that is capable of specifically binding an antigen, or an epitope of an antigen, in the absence of an additional antibody domain. Single-domain antibodies include, for example, V_{H} domain antibodies, V_{NAR} antibodies, camelid V_{H}H antibodies, and V_{L} domain antibodies. V_{NAR} antibodies are produced by cartilaginous fish, such as nurse sharks, wobbegong sharks, spiny dogfish and bamboo sharks. Camelid V_{H}H antibodies are produced by several species including camel, llama, alpaca, dromedary, and guanaco, which produce heavy chain antibodies that are naturally devoid of light chains.

A “monoclonal antibody” is an antibody produced by a single clone of lymphocytes or by a cell into which the coding sequence of a single antibody has been transfected. Monoclonal antibodies include humanized monoclonal antibodies.

A “chimeric antibody” has framework residues from one species, such as human, and CDRs (which generally confer antigen binding) from another species.

A “humanized” antibody is an immunoglobulin including a human framework region and one or more CDRs from a non-human (for example a mouse, rabbit, rat, shark or synthetic) immunoglobulin. The non-human immunoglobulin providing the CDRs is termed a “donor,” and the human immunoglobulin providing the framework is termed an “acceptor.” In one embodiment, all CDRs are from the donor immunoglobulin in a humanized immunoglobulin. Constant regions need not be present, but if they are, they may be substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. A humanized antibody binds to the same antigen as the donor antibody that provides the CDRs. Humanized or other monoclonal antibodies can have additional conservative amino acid substitutions which have substantially no effect on antigen binding or other immunoglobulin functions.

Some examples include portions of antibodies, such as Fab fragments, Fab′ fragments, F(ab)′₂ fragments, single chain Fv proteins (“scFv”), and disulfide stabilized Fv proteins (“dsFv”).

Binding affinity/Specifically binds/Specific binding affinity/Specifically targets: Describes the binding of one molecule to another at greater binding affinity than background binding. Includes the affinity of an antibody for an antigen. In one embodiment, affinity is calculated by a modification of the Scatchard method described by Frankel et al., Mol. Immunol., 16:101-106, 1979. In another embodiment, binding affinity is measured by an antigen/antibody dissociation rate. In another embodiment, a high binding affinity is measured by a competition radioimmunoassay. In another embodiment, binding affinity is measured by ELISA. In another embodiment, antibody affinity is measured by flow cytometry. An antibody that “specifically binds” an antigen (such as PDHK1, PGC-1α, or PDP) is an antibody that binds the antigen with high affinity and does not significantly bind other unrelated antigens.

In some examples, an antibody or fragment thereof (such as an anti-PDHK1, anti-PGC-1α, or anti-PDP antibody provided herein) specifically binds to a target (such as a PDHK1, PGC-1α, or PDP) with a binding constant that is at least 10³ M⁻¹ greater, 10⁴M⁻¹ greater or 10⁵ M⁻¹ greater than a binding constant for other molecules in a sample or subject. In some examples, an antibody (e.g., monoclonal antibody) or fragments thereof, has an equilibrium constant (Kd) of 10 nM or less, such as 9 nM or less, 8.1 nM or less, 8 nM or less, 7 nM or less, 6 nM or less, 6.5 nM or less, 6.3 nM or less, 5 nM or less, 4.3 nM or less, 4 nM or less, 3 nM or less, 2 nM or less, 1.5 nM or less, 1.5 nM or less, 1.4 nM or less, 1.3 nM or less, or 1.2 nM or less. For example, an antibody or fragment thereof binds to a target, such as PDHK1 or PDP with a binding affinity of at least about 0.1×10⁻⁸ M, at least about 0.3×10⁻⁸ M, at least about 0.5×10⁻⁸ M, at least about 0.75×10⁻⁸ M, at least about 1.0×10⁻⁸ M, at least about 1.3×10⁻⁸ M at least about 1.5×10⁻⁸M, or at least about 2.0×10⁻⁸ M, at least about 2.5×10⁻⁸, at least about 3.0×10⁻⁸, at least about 3.5×10⁻⁸, at least about 4.0×10⁻⁸, at least about 4.5×10⁻⁸, at least about 5.0×10⁻⁸ M, at least about 1×10⁻⁹ M, at least about 1.3×10⁻⁹ M, at least about 1.5×10⁻⁹ M, at least about 2×10⁻⁹ M, at least about 3×10⁻⁹ M, at least about 4×10⁻⁹ M, at least about 4.3×10⁻⁹ M, at least about 5×10⁻⁹ M, at least about 6×10⁻⁹ M, at least about 6.3×10⁻⁹ M, at least about 6.9×10⁻⁹ M, at least about 7×10⁻⁹ M, at least about 8×10⁻⁹ M, at least about 8.1×10⁻⁹ M, or at least about 10×10⁻⁹ M. In certain embodiments, a specific binding agent that binds to its target has a dissociation constant (Kd) of ≤100 nM, ≤10 nM, ≤9 nM, ≤8 nM, ≤7 nM, ≤6.9 nM, ≤6.5 nM, ≤6.3 nM, ≤5 nM, ≤4 nM, ≤4.5 nM, ≤3 nM, ≤2 nM, ≤1.5 nM, ≤1 nM, ≤0.1 nM, ≤0.01 nM, or ≤0.001 nM (e.g., 10⁻⁸M or less, e.g., from 10⁻⁸M to 10⁻¹³M, e.g., from 10⁻⁹ M to 10⁻¹³ M). In one embodiment, Kd is measured by a radiolabeled antigen binding assay (RIA) performed with the Fab version of an antibody of interest and its antigen (see, e.g., Chen et al., J. Mol. Biol. 293:865-881, 1999). In another example, Kd is measured using surface plasmon resonance assays using a BIACORES-2000 or a BIACORES-3000 (BIAcore, Inc., Piscataway, N.J.) at 25 °C with immobilized antigen CM5 chips at about 10 response units (RU).

Also includes the affinity of a binding domain (or a TCR, CAR or DARIC comprising a binding domain or a fusion protein containing a binding domain) to its target. For example, a binding domain (or a TCR, CAR or DARIC comprising a binding domain or a fusion protein containing a binding domain) “specifically binds” to a target molecule if it binds to or associates with a target molecule with an affinity or Kₐ (i.e., an equilibrium association constant of a particular binding interaction with units of 1/M) of, for example, greater than or equal to about 10⁵ M⁻¹. In certain embodiments, a binding domain (or a fusion protein thereof) binds to a target with a Kₐ greater than or equal to about 10⁶ M⁻¹, 10⁷ M⁻¹, 10⁸ M⁻¹, 10⁹ M⁻¹, 10¹⁰ M⁻¹, 10¹¹ M⁻¹, 10¹² M⁻¹, or 10¹³ M⁻¹. “High affinity” binding domains (or single chain fusion proteins thereof) refers to those binding domains with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, at least 10¹³ M⁻¹, or greater. Alternatively, affinity may be defined as an equilibrium dissociation constant (K_{d}) of a particular binding interaction with units of M (e.g., 10⁻⁵ M to 10⁻¹³ M, or less). Affinities of binding domain polypeptides and TCR, CAR or DARIC proteins according to the present disclosure can be determined using conventional techniques, e.g., by competitive ELISA (enzyme-linked immunosorbent assay), or by binding association, or displacement assays using labeled ligands, or using a surface-plasmon resonance device such as the Biacore T100, which is available from Biacore, Inc., Piscataway, NJ, or optical biosensor technology such as the EPIC system or EnSpire that are available from Corning and Perkin Elmer respectively (see also, e.g., Scatchard et al. (1949) Ann. N.Y. Acad. Sci. 51:660; and U.S. Pat. Nos. 5,283,173; 5,468,614, or the equivalent).

In one embodiment, the affinity of specific binding is about 2 times greater than background binding, about 5 times greater than background binding, about 10 times greater than background binding, about 20 times greater than background binding, about 50 times greater than background binding, about 100 times greater than background binding, or about 1000 times greater than background binding or more.

Antigen (Ag): A compound, composition, or substance that can stimulate the production of antibodies or a T cell response in an animal, including compositions (such as one that includes a tumor-specific protein) that are injected or absorbed into an animal. An antigen reacts with the products of specific humoral or cellular immunity, including those induced by heterologous antigens, such as the disclosed antigens. A “target antigen” or “target antigen of interest” is an antigen that a binding domain of a TCR, CAR or DARIC contemplated herein, is designed to bind.

Autoimmune disease: A disease in which the body produces an immunogenic (i.e., immune system) response to some constituent of its own tissue. In other words, the immune system loses its ability to recognize some tissue or system within the body as “self” and targets and attacks it as if it were foreign. Autoimmune diseases can be classified into those in which predominantly one organ is affected (e.g., hemolytic anemia and anti-immune thyroiditis), and those in which the autoimmune disease process is diffused through many tissues (e.g., systemic lupus erythematosus). For example, multiple sclerosis is thought to be caused by T cells attacking the sheaths that surround the nerve fibers of the brain and spinal cord. This results in loss of coordination, weakness, and blurred vision. Autoimmune diseases are known in the art and include, for instance, Hashimoto's thyroiditis, Grave's disease, lupus, multiple sclerosis, rheumatic arthritis, hemolytic anemia, anti-immune thyroiditis, systemic lupus erythematosus, celiac disease, Crohn's disease, colitis, diabetes, scleroderma, psoriasis, and the like.

Autologous: With respect to cells, refers to cells from the same subject.

Benign/non-malignant: Tumors that may grow larger but do not spread to other parts of the body. Benign tumors are self-limited and typically do not invade or metastasize.

Cancer: A class of diseases or conditions in which abnormal cells divide without control and can invade nearby tissues. A malignant cancer is one in which a group of tumor cells display one or more of uncontrolled growth (i.e., division beyond normal limits), invasion (i.e., intrusion on and destruction of adjacent tissues), and metastasis (i.e., spread to other locations in the body via lymph or blood). As used herein, the term “metastasize” refers to the spread of cancer from one part of the body to another. A tumor formed by cells that have spread is called a “metastatic tumor” or a “metastasis.” The metastatic tumor contains cells that are like those in the original (primary) tumor. A “cancer cell” or “tumor cell” refers to an individual cell of a cancerous growth or tissue. A tumor refers generally to a swelling or lesion formed by an abnormal growth of cells, which may be benign, pre-malignant, or malignant. Most cancers form tumors, but some, e.g., leukemia, do not necessarily form tumors. For those cancers that form tumors, the terms cancer (cell) and tumor (cell) are used interchangeably. The amount of a tumor in an individual is the “tumor burden” which can be measured as the number, volume, or weight of the tumor.

Exemplary tumors, such as cancers, that can be treated using the disclosed immune effector cells include solid tumors, such as breast carcinomas (e.g. lobular and duct carcinomas, such as a triple negative breast cancer), sarcomas, carcinomas of the lung (e.g., non-small cell carcinoma, large cell carcinoma, squamous carcinoma, and adenocarcinoma), mesothelioma of the lung, colorectal adenocarcinoma, stomach carcinoma, prostatic adenocarcinoma, ovarian carcinoma (such as serous cystadenocarcinoma and mucinous cystadenocarcinoma), ovarian germ cell tumors, testicular carcinomas and germ cell tumors, pancreatic adenocarcinoma, biliary adenocarcinoma, hepatocellular carcinoma, bladder carcinoma (including, for instance, transitional cell carcinoma, adenocarcinoma, and squamous carcinoma), renal cell adenocarcinoma, endometrial carcinomas (including, e.g., adenocarcinomas and mixed Mullerian tumors (carcinosarcomas)), carcinomas of the endocervix, ectocervix, and vagina (such as adenocarcinoma and squamous carcinoma of each of same), tumors of the skin (e.g., squamous cell carcinoma, basal cell carcinoma, malignant melanoma, skin appendage tumors, Kaposi sarcoma, cutaneous lymphoma, skin adnexal tumors and various types of sarcomas and Merkel cell carcinoma), esophageal carcinoma, carcinomas of the nasopharynx and oropharynx (including squamous carcinoma and adenocarcinomas of same), salivary gland carcinomas, brain and central nervous system tumors (including, for example, tumors of glial, neuronal, and meningeal origin), tumors of peripheral nerve, soft tissue sarcomas and sarcomas of bone and cartilage, head and neck squamous cell carcinoma, and lymphatic tumors (including B-cell and T-cell malignant lymphoma). In one example, the tumor is an adenocarcinoma.

The disclosed immune effector cells can also be used to treat liquid tumors, such as a lymphatic, white blood cell, or other type of leukemia. In a specific example, the tumor treated is a tumor of the blood, such as a leukemia (for example acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia (T-PLL), large granular lymphocytic leukemia, and adult T-cell leukemia), a lymphoma (such as Hodgkin's lymphoma or non-Hodgkin's lymphoma), or a myeloma.

Chimeric antigen receptor (CAR): A chimeric molecule that includes an antigen-binding portion (such as a single domain antibody or scFv) and a signaling domain, such as a signaling domain from a T cell receptor (e.g., CD3 D). Typically, CARs include an antigen-binding portion, a transmembrane domain, and an intracellular domain. The intracellular domain typically includes a signaling chain having an immunoreceptor tyrosine-based activation motif (ITAM), such as CD3□ or Fc□RI□. In some instances, the intracellular domain also includes the intracellular portion of at least one additional co-stimulatory domain, such as CD28, 4-1BB (CD137), ICOS, OX40 (CD134), CD27 and/or DAP10.

Contact: Placement in direct physical association, including a solid or a liquid form. Contacting can occur in vitro or ex vivo, for example, by adding a reagent to a sample (such as one containing immune effector cells), or in vivo by administering to a subject.

Costimulatory signal: A signal, which in combination with a primary signal, such as TCR/CD3 ligation, leads to immune effector cell (e.g., T cell) proliferation, cytokine production, and/or upregulation or downregulation of particular molecules (e.g., CD28).

Costimulatory ligand: A molecule that binds a costimulatory molecule. A costimulatory ligand may be soluble or provided on a surface. A “costimulatory molecule” refers to the cognate binding partner on an immune effector cell (e.g., T cell) cell that specifically binds with a costimulatory ligand (e.g., anti-CD28 antibody).

Decrease/lower/lessen/reduce/abate: Refers generally to the ability of composition contemplated herein to produce, elicit, or cause a lesser physiological response (i.e., downstream effects) compared to the response caused by either vehicle or a control molecule/composition. A “decrease” or “reduced” amount is typically a “statistically significant” amount, and may include an decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response (reference response) produced by vehicle, a control composition, or the response in a particular cell lineage.

Differentiation: A method of decreasing the potency or proliferation of a cell or moving the cell to a more developmentally restricted state. In particular embodiments, differentiated T cells acquire immune effector cell functions.

Effective amount/therapeutically effective amount): The amount of an agent (such as a genetically modified therapeutic cell, e.g., immune effector cell (e.g., T cell), disclosed herein, or other anti-cancer agents) that is sufficient to effect beneficial or desired therapeutic result, including clinical results.

In some examples, a “prophylactically effective amount” refers to an amount of a genetically modified therapeutic cell, e.g., T cell, disclosed herein, effective to achieve the desired prophylactic result. Typically but not necessarily, since a prophylactic dose is used in subjects prior to or at an earlier stage of disease, the prophylactically effective amount is less than the therapeutically effective amount.

An effective amount may vary depending upon one or more of: the subject and disease condition being treated, the sex, weight and age of the subject, the severity of the disease condition, the manner of administration, the ability of the T cells to elicit a desired response in the individual, and the like. The beneficial therapeutic effect can include enablement of diagnostic determinations; amelioration of a disease, symptom, disorder, or pathological condition; reducing or preventing the onset of a disease, symptom, disorder or condition; and generally counteracting a disease, symptom, disorder or pathological condition.

An effective amount can be one in which any toxic or detrimental effects of the virus or transduced therapeutic cells are outweighed by the therapeutically beneficial effects. The term “effective amount” includes an amount that is effective to “treat” a subject (e.g., a patient). When a therapeutic amount is indicated, the precise amount of the compositions of the present disclosure to be administered can be determined by a physician with consideration of individual differences in age, weight, tumor size, extent of infection or metastasis, and condition of the patient (subject).

In one embodiment, an “effective amount” (e.g., of an immune effector cell (e.g., T cell) disclosed herein) may be an amount sufficient to reduce the volume/size of a tumor, the weight of a tumor, the number/extent of metastases, reduce the volume/size of a metastasis, the weight of a metastasis, or combinations thereof, for example by at least about 10%, at least about 20%, at least about 25%, at least about 50%, at least about 70%, at least about 75%, at least about 80%, at least about 90%, at least about 95%, or at least about 99% (as compared to no administration of the therapeutic agent or compared to transduced immune effector cells cultured in the absence of the one or more of the metabolic enhancers). In one embodiment, an “effective amount” (e.g., of an immune effector cell (e.g., T cell) disclosed herein) may be an amount sufficient to increase the survival time of a subject, such as a subject with cancer, for example by at least about 10%, at least about 20%, at least about 25%, at least about 50%, at least about 70%, at least about 75%, at least about 80%, at least about 90%, at least about 95%, 100%, 200%, 300%, 400%, or 500% (as compared to no administration of the therapeutic agent or compared to transduced immune effector cells cultured in the absence of the one or more of the metabolic enhancers). In one embodiment, an “effective amount” (e.g., of a metabolic enhancer, such as PDHK1 inhibitor, PGC1α polypeptide or variants thereof, PGC1α agonists and/or activator of PDP) may be an amount sufficient to increase the respiratory capacity of the transduced immune effector cells (e.g., T cells), for example by at least about 10%, at least about 20%, at least about 25%, at least about 50%, at least about 70%, at least about 75%, at least about 80%, at least about 90%, at least about 95%, at least about 99%, at least about 100%, at least about 200%, at least about 300%, at least about 400%, at least about 500%, or at least about 600% (as compared to no administration of the therapeutic agent or compared to transduced immune effector cells cultured in the absence of the one or more of the metabolic enhancers). In one embodiment, an “effective amount” (e.g., of a metabolic enhancer, such as PDHK1 inhibitor, PGC1α polypeptide or variants thereof, PGC1α agonists and/or activator of PDP) may be an amount sufficient to increase the mitochondrial mass of the transduced immune effector cells (e.g., T cells), for example by at least about 25%, at least about 30%, at least about 40%, or at least about 50% (as compared to no administration of the therapeutic agent or compared to transduced immune effector cells cultured in the absence of the one or more of the metabolic enhancers).

Enhance/promote/increase/expand: Refers generally to the ability of a composition contemplated herein to produce, elicit, or cause a greater physiological response (i.e., downstream effects) compared to the response caused by either vehicle or a control molecule/composition. A measurable physiological response may include an increase in immune effector cell (e.g., T cell) expansion, activation, persistence, respiratory capacity, and/or an increase in cancer cell death killing ability, among others apparent from the understanding in the art and the description herein. An “increased” or “enhanced” amount is typically a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response produced by vehicle or a control composition.

Epitope/antigenic determinant: The region of an antigen to which a binding agent binds.

ex vivo: Refers generally to activities that take place outside an organism, such as experimentation or measurements done in or on living tissue in an artificial environment outside the organism, for example with minimum alteration of the natural conditions. In particular embodiments, “ex vivo” procedures involve living cells or tissues taken from an organism and cultured or modulated in a laboratory apparatus, usually under sterile conditions, and typically for a few hours or up to about 24 hours, but including up to 48 or 72 hours, depending on the circumstances. In certain embodiments, such tissues or cells can be collected and frozen, and later thawed for ex vivo treatment. Tissue culture experiments or procedures lasting longer than a few days using living cells or tissue are typically considered to be “in vitro,” though in certain embodiments, this term can be used interchangeably with ex vivo.

Genetically engineered/genetically modified: Refers to the addition of extra genetic material in the form of DNA or RNA into the total genetic material in a cell. The terms, “genetically modified cells,” “modified cells,” “transformed cells,” and, “redirected cells,” are used interchangeably.

Gene therapy: Refers to the introduction of extra genetic material in the form of DNA or RNA into the total genetic material in a cell that restores, corrects, or modifies expression of a gene, or for the purpose of expressing a therapeutic polypeptide, e.g., a TCR, DARIC, or CAR and/or one or more cytokines. In particular embodiments, immune effector cells (e.g., T cells) are modified to express an engineered TCR, DARIC, or CAR without modifying the genome of the cells, e.g., by introducing an episomal vector that expresses the TCR, DARIC or CAR into the cell.

in vivo: Refers generally to activities that take place inside an organism, such as cell self-renewal and expansion of cells. In one embodiment, the term “in vivo expansion” refers to the ability of a cell population to increase in number in vivo.

Infectious disease: A disease that can be transmitted from person to person or from organism to organism, and is caused by a microbial agent (e.g., virus, bacterium, fungus, or parasite). Examples of infectious diseases include hepatitis, sexually transmitted diseases (e.g., Chlamydia, gonorrhea, HPV), tuberculosis, HIV/AIDS, diphtheria, hepatitis B, hepatitis C, cholera, SARS-Covid 19, and influenza.

In some examples, an infectious disease is caused by positive-strand RNA viruses and negative-strand RNA viruses. Exemplary positive-strand RNA viruses include, but are not limited to: Picornaviruses (such as Aphthoviridae [for example foot-and-mouth-disease virus (FMDV)]), Cardioviridae; Enteroviridae (such as Coxsackie viruses, Echoviruses, Enteroviruses, and Polioviruses); Rhinoviridae (Rhinoviruses)); Hepataviridae (Hepatitis A viruses); Togaviruses (examples of which include rubella; alphaviruses (such as Western equine encephalitis virus, Eastern equine encephalitis virus, and Venezuelan equine encephalitis virus)); Flaviviruses (examples of which include Dengue virus, West Nile virus, and Japanese encephalitis virus); Calciviridae (which includes Norovirus and Sapovirus); and Coronaviruses (examples of which include SARS coronaviruses, such as the Urbani strain, SARS-CoV-1 and SARS-CoV-2). Exemplary negative-strand RNA viruses include, but are not limited to: Orthomyxyoviruses (such as the influenza virus), Rhabdoviruses (such as Rabies virus), and Paramyxoviruses (examples of which include measles virus, respiratory syncytial virus, and parainfluenza viruses).

In some examples, an infectious disease is caused by a DNA virus. Exemplary DNA viruses include, but are not limited to: Herpesviruses (such as Varicella-zoster virus, for example the Oka strain; cytomegalovirus; and Herpes simplex virus (HSV) types 1 and 2), Adenoviruses (such as Adenovirus type 1 and Adenovirus type 41), Poxviruses (such as Vaccinia virus), and Parvoviruses (such as Parvovirus B19).

In some examples, an infectious disease is caused by a Retrovirus. Examples of retroviruses include, but are not limited to: human immunodeficiency virus type 1 (HIV-1), such as subtype C; HIV-2; equine infectious anemia virus; feline immunodeficiency virus (FIV); feline leukemia viruses (FeLV); simian immunodeficiency virus (SIV); and avian sarcoma virus.

In one example, the virus is one or more of the following: HIV-1 (for example an HIV antibody, p24 antigen, or HIV genome); Hepatitis A virus (for example an Hepatitis A antibody, or Hepatitis A viral genome); Hepatitis B (HB) virus (for example an HB core antibody, HB surface antibody, HB surface antigen, or HB viral genome); Hepatitis C (HC) virus (for example an HC antibody, or HC viral genome); Hepatitis D (HD) virus (for example an HD antibody, or HD viral genome); Hepatitis E virus (for example a Hepatitis E antibody, or HE viral genome); a respiratory virus (such as influenza A & B, respiratory syncytial virus, human parainfluenza virus, a cornonavirus such as SARS-CoV-1 or SARS-CoV-2 (including variants thereof, such as alpha (B.1.1.7 and Q lineages); beta (B.1.351 and descendent lineages); delta (B.1.617.2 and AY lineages); gamma (P.1 and descendent lineages); epsilon (B.1.427 and B.1.429); eta (B.1.525); iota (B.1.526); kappa (B.1.617.1); 1.617.3; mu (B.1.621, B.1.621.1) and zeta (P.2)) or human metapneumovirus), or West Nile Virus.

In some examples, an infectious disease is caused by a bacterium. Bacteria can be classified as gram-negative or gram-positive. Exemplary gram-negative bacteria include, but are not limited to: Escherichia coli (e.g., K-12 and O157:H7), Shigella dysenteriae, and Vibrio cholerae. Exemplary target gram-positive bacteria include, but are not limited to: Bacillus anthracis, Staphylococcus aureus, Listeria, pneumococcus, gonococcus, and streptococcal meningitis. In one example, the bacteria causing an infectious disease is one or more of the following: Group A Streptococcus; Group B Streptococcus; Helicobacter pylori; Methicillin-resistant Staphylococcus aureus; vancomycin-resistant enterococci; Clostridium difficile; E. coli (e.g., Shiga toxin producing strains); Listeria; Salmonella; Campylobacter; B. anthracis (such as spores); Chlamydia trachomatis; Ebola, and Neisseria gonorrhoeae.

In some examples, an infectious disease is caused by a protozoa, nemotode, or fungi. Exemplary protozoa include, but are not limited to, Plasmodium (e.g., Plasmodium falciparum: to diagnose malaria), Leishmania, Acanthamoeba, Giardia, Entamoeba, Cryptosporidium, Isospora, Balantidium, Trichomonas, Trypanosoma (e.g., Trypanosoma brucei), Naegleria, and Toxoplasma. Exemplary fungi include, but are not limited to, Coccidiodes immitis and Blastomyces dermatitidis.

Immune effector cell: Any cell of the immune system that has one or more effector functions (e.g., cytotoxic cell killing activity, secretion of cytokines, induction of ADCC and/or CDC). Examples include T lymphocytes, in particular cytotoxic T cells (CTLs; CD8⁺ T cells), TILs, natural killer (NK) cells, and helper T cells (HTLs; CD4⁺ T cells). Specific examples of immune effector cell include but are not limited to: αβ-T cells, γδ-T cells, and natural killer T (NKT) cells. In some examples an immune effector cell population used in the disclosed methods is purified.

Immunodeficiency: The state of a patient whose immune system has been compromised by disease or by administration of chemicals. This condition makes the system deficient in the number and type of blood cells needed to defend against a foreign substance. Immunodeficiency conditions or diseases are known in the art and include, for example, AIDS (acquired immunodeficiency syndrome), SCID (severe combined immunodeficiency disease), selective IgA deficiency, common variable immunodeficiency, X-linked agammaglobulinemia, chronic granulomatous disease, hyper-IgM syndrome, and diabetes.

Isolated: An “isolated” biological component (such as an immune effector cell (e.g., T cell), a nucleic acid molecule, or a protein) has been substantially separated, produced apart from, or purified away from other biological components in the cell or tissue of an organism in which the component occurs, such as other cells (e.g., RBCs), chromosomal and extrachromosomal DNA and RNA, and proteins. Nucleic acids and proteins that have been “isolated” include nucleic acids and proteins purified by standard purification methods. The term also embraces nucleic acids and proteins prepared by recombinant expression in a host cell as well as chemically synthesized nucleic acids and proteins. In some examples, an “isolated peptide” or an “isolated polypeptide” and the like refer to in vitro isolation and/or purification of a peptide or polypeptide molecule from a cellular environment, and from association with other components of the cell, i.e., it is not significantly associated with in vivo substances. Similarly, an “isolated cell” refers to a cell that has been obtained from an in vivo tissue or organ and is substantially free of extracellular matrix. An “isolated polynucleotide” in some examples refers to a polynucleotide that has been purified from the sequences which flank it in a naturally-occurring state, e.g., a DNA fragment that has been removed from the sequences that are normally adjacent to the fragment. An “isolated polynucleotide” can also refer to a complementary DNA (cDNA), a recombinant DNA, or other polynucleotide that does not exist in nature and that has been made by the hand of man. Isolated cells, nucleic acid molecules, or proteins in some examples are at least 50% pure, such as at least 75%, at least 80%, at least 90%, at least 95%, at least 98%, or at least 100% pure.

Maintain/preserve/maintenance/no change/no substantial change/no substantial decrease: Refers generally to the ability of a composition contemplated herein to produce, elicit, or cause a comparable physiological response (i.e., downstream effects) in a cell, as compared to the response caused by either vehicle, a control molecule/composition, or the response in a particular cell lineage. A comparable response is one that is not significantly different or measurable different from the reference response.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence (such as a PDHK1, PGC1α, or PDP coding sequence). Generally, operably linked sequences are contiguous and, where necessary to join two protein coding regions, in the same reading frame.

Peroxisome proliferator-activated receptor gamma coactivator 1-alpha (PGC1α): (e.g., OMIM 604517): A transcriptional coactivator of nuclear receptors and other transcription factors that regulate metabolic processes, including mitochondrial biogenesis and respiration, hepatic gluconeogenesis, and muscle fiber-type switching.

Human PGC1α is encoded by the PPARGC1A gene at chromosome 4p15. PGC1α sequences are publicly available, for example from the GenBank® sequence database (e.g., Accession Nos. Q9UBK2.1, NP_001341754.1, NP_112637.1, NP_032930.1 and NP_999128.2 provide exemplary PGC1α protein sequences, while Accession Nos. NM_001354825.2, NM_013261.5, NM_008904.2 and NM_213963.2 provide exemplary PGC1α nucleic acid sequences). One of ordinary skill in the art can identify additional PGC1α nucleic acid and protein sequences, including PGC1α variants, such as those having at least 80%, at least 85%, at least 90%, at least 92%, at least 95%, at least 98%, or at least 99% sequence identity to these GenBank® sequences, such as at least 80%, at least 85%, at least 90%, at least 92%, at least 95%, at least 96%, at least 97%, at least 98%, or at least 99% sequence identity to SEQ ID NO: 18, 19, aa 1-270 of SEQ ID NO: 18 or 19, or aa 1-290 of SEQ ID NO: 18 or 19.

Pharmaceutically acceptable carriers: The pharmaceutically acceptable carriers useful in this disclosure are conventional. Remington's Pharmaceutical Sciences, by E. W. Martin, Mack Publishing Co., Easton, PA, 15th Edition (1975), describes compositions and formulations suitable for pharmaceutical delivery of a therapeutic agent, such as an immune effector cell (e.g., T cell) made using the methods disclosed herein.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Prevent: An approach for preventing, inhibiting, or reducing the likelihood of the occurrence or recurrence of, a disease or condition, e.g., cancer. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

Proliferation: An increase in cell division, either symmetric or asymmetric division of cells. In particular embodiments, “proliferation” refers to the symmetric or asymmetric division of immune effector cells (e.g., T cells). “Increased proliferation” occurs when there is an increase in the number of cells in a treated sample compared to cells in a non-treated sample.

Promoter: An array of nucleic acid control sequences which direct transcription of a nucleic acid. A promoter includes necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription.

Examples of promoters that can used with the disclosed methods include, but are not limited to viral promoters, such as 7.5 promoter, SV40 promoter, CMV enhancer-promoter, and the CMV enhancer/β-actin promoter. Both constitutive and inducible promoters can be used (see e.g., Bitter et al., Methods in Enzymology 153:516-544, 1987). Also included are those promoter elements which are sufficient to render promoter-dependent gene expression controllable for cell-type specific, tissue-specific, or inducible by external signals or agents; such elements may be located in the 5′ or 3′ regions of the gene. Promoters produced by recombinant DNA or synthetic techniques can also be used to provide for transcription of the nucleic acid sequences.

Pyruvate dehydrogenase kinase 1 (PDHK1 or PDK1): (e.g., OMIM 602524): EC 2.7.11.2. A member of a family of pyruvate dehydrogenase (PDH) kinases that reversibly inactivates the mitochondrial PDH complex by ATP-dependent serine phosphorylation of the alpha subunit of the complex's E1 component. By downregulating the activity of PDH complex, PDHK1 decreases the oxidation of pyruvate in mitochondria and increases the conversion of pyruvate to lactate in the cytosol.

Human PDHK1 is encoded by the PDK1 gene on chromosome 2, and the native protein is 436 amino acids. PDHK1 sequences are publicly available, for example from the GenBank® sequence database (e.g., Accession Nos. NP_002601.1 and AAC42009.1 (mature peptide aa 29-436), NP_001265478.11 (mature peptide aa 29-456), NP_001346931.1, and NP_446278.2 (mature peptide aa 27-434) provide exemplary PDHK1 protein sequences, while Accession Nos. BC039158.1 NM_002610.5 (coding sequence 62-1372), NR 103731.1, NM_001360002.1 (coding sequence nt 105-1409) and NM_053826.2 (coding sequence nt 105-1409) provide exemplary PDHK1 nucleic acid sequences). One of ordinary skill in the art can identify additional PDHK1 nucleic acid and protein sequences, including PDHK1 variants, such as those having at least 80%, at least 85%, at least 90%, at least 92%, at least 95%, at least 98%, or at least 99% sequence identity to these GenBank® sequences, such as at least 80%, at least 85%, at least 90%, at least 92%, at least 95%, at least 96%, at least 97%, at least 98%, or at least 99% sequence identity to SEQ ID NO: 1,2, nt 44-1354 of SEQ ID NO: 1, or aa 29-436 of SEQ ID NO: 2.

Pyruvate dehydrogenase phosphatase (PDP): (e.g., OMIM 605993): EC 3.1.3.43. Also called PDP1, PDH, PDPC, PPM2A, and PPM2C. An enzyme that serves to reverse the effects of pyruvate dehydrogenase kinase upon pyruvate dehydrogenase. In humans, catalytic subunit 1 of PDP is encoded by the PDP1 gene on chromosome 8. In humans, isoform 1 is 537 aa, while isoform 2 is 562 aa.

PDP sequences are publicly available, for example from the GenBank® sequence database (e.g., Accession Nos. NP_060914.2, NP_001155253.1 NP_001155251.1, NP_001277320.1, NP_001028625.1, NP_001091701.1, AAC40167.1 and AAC40168.1 provide exemplary PDP protein sequences, while Accession Nos. NM_018444.4 (coding sequence nt 202-1815), NM_001161779.2, (coding sequence 301-1989), NM 001161780.2 (coding sequence 162-1850), NM_001098231.1 (coding sequence nt 37-1761), AF062741.1 (coding sequence nt 28-1620) and NM_001161781.2 (coding sequence 341-1954) provide exemplary PDP nucleic acid sequences). One of ordinary skill in the art can identify additional PDP nucleic acid and protein sequences, including PDP variants, such as those having at least 80%, at least 85%, at least 90%, at least 92%, at least 95%, at least 98%, or at least 99% sequence identity to these GenBank® sequences, such as at least 80%, at least 85%, at least 90%, at least 92%, at least 95%, at least 96%, at least 97%, at least 98%, or at least 99% sequence identity to SEQ ID NO: 3, 4, or nt 202-1815 of SEQ ID NO: 3.

Recombinant: A recombinant nucleic acid molecule is one that has a sequence that is not naturally occurring (e.g., a human PDP or PGC1α coding sequence operably linked to a non-human promoter) or has a sequence that is made by an artificial combination of two otherwise separated segments of sequence. In some examples, this artificial combination is accomplished by chemical synthesis or by the artificial manipulation of isolated segments of nucleic acids, such as by genetic engineering techniques. Similarly, a recombinant protein is one encoded by a recombinant nucleic acid molecule. Similarly, a recombinant or transgenic cell is one that contains a recombinant nucleic acid molecule and expresses a recombinant protein.

Sequence identity: The similarity between amino acid (or nucleotide) sequences is expressed in terms of the similarity between the sequences, otherwise referred to as sequence identity. Sequence identity is frequently measured in terms of percentage identity (or similarity or homology); the higher the percentage, the more similar the two sequences are.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, MD) and on the internet, for use in connection with the sequence analysis programs blastp, blastn, blastx, tblastn and tblastx. A description of how to determine sequence identity using this program is available on the NCBI website on the internet.

Variants of native protein or coding sequences are typically characterized by possession of at least about 80%, at least 90%, at least 95%, at least 96%, at least 97%, at least 98% or at least 99% sequence identity counted over the full-length alignment with the amino acid sequence using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or at least 95% depending on their similarity to the reference sequence. Methods for determining sequence identity over such short windows are available at the NCBI website on the internet. These sequence identity ranges are provided for guidance only; it is possible that strongly significant homologs could be obtained that fall outside of the ranges provided.

Thus, a PDHK1, PGC1α, and/or PDP protein or nucleic acid sequence can have at least 80%, at least 85%, at least 90%, at least 91%, at least 92%, at least 93%, at least 94%, at least 95%, at least 96%, at least 97%, at least 98% or at least 99% sequence identity to any of the sequences shown in the GenBank® Accession Nos. provided herein (such as to SEQ ID NO: 1, 2, 3, 4, 18, 19, nt 44-1354 of SEQ ID NO: 1, aa 29-436 of SEQ ID NO 2, nt 202-1815 of SEQ ID NO: 3, aa 1-270 of SEQ ID NO: 18, aa 1-270 of SEQ ID NO: 19, aa 1-290 of SEQ ID NO: 18, or aa 1-290 of SEQ ID NO: 19).

Stimulation: A primary response induced by binding of a stimulatory molecule (e.g., a TCR/CD3 complex) with its cognate ligand thereby mediating a signal transduction event including, but not limited to, signal transduction via the TCR/CD3 complex. A “stimulatory molecule,” refers to a molecule on an immune effector cell (e.g., T cell) that specifically binds with a cognate stimulatory ligand. A “stimulatory ligand,” as used herein, means a ligand that when present on an antigen presenting cell (e.g., an artificial APC (aAPC), a dendritic cell, a B-cell, and the like) can specifically bind with a cognate binding partner (referred to herein as a “stimulatory molecule”) on an immune effector cell (e.g., T cell), thereby mediating a primary response by the immune effector cell (e.g., T cell), including, but not limited to, activation, initiation of an immune response, proliferation, and the like. Stimulatory ligands include, but are not limited to CD3 ligands, e.g., an anti-CD3 antibody and CD2 ligands, e.g., anti-CD2 antibody, and peptides, e.g., CMV, HPV, EBV peptides.

Subject/Individual/Patient: A vertebrate, such as a mammal, for example a human. Mammals include, but are not limited to, murines, simians, humans, farm animals, sport animals, and pets. In one embodiment, the subject is a non-human mammalian subject, such as a monkey or other non-human primate, mouse, rat, rabbit, guinea pig, pig, goat, sheep, dog, cat, horse, or cow. In some examples, the subject has a tumor, such as a cancer, that can be treated using the immune effector cells (e.g., T cells) disclosed herein. In some examples, the subject is a laboratory animal/organism, such as a mouse, rabbit, guinea pig, or rat. In some examples, is any animal that exhibits a symptom of a cancer that can be treated with the gene therapy vectors, cell-based therapeutics, and methods disclosed herein. In one example a subject includes farm animals and domestic animals or pets (such as a cat or dog). In one example, a subject is a human patient that has a cancer, has been diagnosed with a cancer, or are at risk or having a cancer. A “patient” can refer to a subject that has been diagnosed with a particular indication that can be treated with the gene therapy vectors, cell-based therapeutics, and methods disclosed elsewhere herein.

Syngeneic: With respect to cells, refers to cells of a different subject that are genetically identical to the cell in comparison.

T cell/T lymphocyte: White blood cells containing a T cell receptor on their cell surface, which play a role in cell-mediated immunity. Include thymocytes, naïve T lymphocytes, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD4⁺ T cell) CD4⁺ T cell, a cytotoxic T cell (CTL; CD8⁺ T cell), a tumor infiltrating cytotoxic T cell (TIL; CD8⁺ T cell), CD4⁺CD8⁺ T cell, CD4-CD8⁺ T cell, or any other subset of T cells. Other illustrative populations of T cells suitable for use in particular embodiments include naïve T cells and memory T cells.

“Potent T cells,” and “young T cells,” are used interchangeably in particular embodiments and refer to T cell phenotypes wherein the T cell is capable of proliferation and a concomitant decrease in differentiation. In particular embodiments, the young T cell has the phenotype of a “naïve T cell.” In various embodiments, the manufacturing methods contemplated herein produce young T cells; cells wherein T cell proliferation has been uncoupled from T cell differentiation during T cell stimulation, activation, and expansion. Without wishing to be bound by any particular theory, the potent T cells manufactured with the compositions and methods contemplates possess greater antitumor efficacy after adoptive transfer. In particular embodiments, young T cells comprise one or more of, or all of the following biological markers: CD62L, CCR7, CD28, CD27, CD122, CD127, CD197, and CD38. In one embodiment, young T cells comprise one or more of, or all of the following biological markers: CD62L, CD127, CD197, and CD38. In one embodiment, the young T cells lack expression of CD57, CD244, CD160, PD-1, CTLA4, TIM3, and LAG3.

“Modified T cells” refer to T cells that have been modified by the introduction of a polynucleotide encoding an engineered TCR, DARIC, or CAR, such as those provided herein. Modified T cells include both genetic and non-genetic modifications (e.g., episomal or extrachromosomal).

T cell manufacturing/methods of manufacturing T cells: Process of producing a therapeutic composition of T cells, which manufacturing methods may include one or more of, or all of the following steps: harvesting, stimulation, activation, and expansion.

Transgene: An exogenous gene supplied by a vector, such as a viral vector. In one example, a transgene includes an engineered T cell receptor (TCR), a dimerizing agent regulated immunoreceptor complex (DARIC), or a chimeric antigen receptor (CAR).

Treating, Treatment, and Therapy: Any success or indicia of success in the attenuation or amelioration of an injury, pathology or condition, including any objective or subjective parameter such as abatement, remission, diminishing of symptoms or making the condition more tolerable to the patient, slowing in the rate of degeneration or decline, making the final point of degeneration less debilitating, improving a subject's physical or mental well-being, or prolonging the length of survival. Treatment does not necessarily indicate complete eradication or cure of the disease or condition, or associated symptoms thereof. The treatment may be assessed by objective or subjective parameters; including the results of a physical examination, blood and other clinical tests (such as imaging), and the like. In some examples, treatment with the disclosed methods results in a decrease in the number, volume, and/or weight of a tumor and/or metastases.

Under conditions sufficient for: A phrase that is used to describe any environment that permits a desired activity. In one example the desired activity is increased respiratory capacity of an immune effector cell (e.g., T cell). In one example the desired activity is treatment of a tumor in vivo, for example using the immune effector cells (e.g., T cells) made using the disclosed methods.

Vector: A nucleic acid molecule as introduced into a host cell (such as a tumor cell or immune effector cell), thereby producing a transformed host cell. A vector may include nucleic acid sequences that permit it to replicate in the host cell, such as an origin of replication. A vector may also include one or more coding sequences, for example in combination other sequences. A vector can transduce, transform or infect a cell, such as an immune effector cell (e.g., T cell), thereby causing the cell to express nucleic acids and/or proteins other than those native to the cell. A vector optionally includes materials to aid in achieving entry of the nucleic acid into the cell, such as a viral particle, liposome, protein coating or the like. Exemplary vectors include plasmids and viral vectors, such as those from herpes simplex virus (HSV), lentivirus, vaccinia virus, adenovirus, poxvirus, reovirus, poliovirus, coxsackie virus, measles virus, vesicular stomatitis virus (VSV), Seneca valley virus, ECHO virus, Newcastle disease virus, chicken anemia virus, and parovirus.

Xenogeneic: With respect to cells, refers to cells of a different species to the cell in comparison. In preferred embodiments, the cells of the disclosure are allogeneic.

### II. Overview

The immunotherapeutic treatment of cancer embodies multiple approaches, including checkpoint blockade with antibodies, oncolytic viruses, and the transfer of tumor-specific T cells into cancer patients. T cells can be genetically modified in culture to be redirected to tumor targets or have greater in vivo function. However, the genetic modification and subsequent expansion of T cells in vitro can cause metabolic stresses that limit their persistence and function in vivo. It is shown herein that by redirecting glucose metabolism using a PDHK1 inhibitor, namely dichloroacetate, promotes mitochondrial health during therapeutic T cell generation. As metabolic stress in culture is limited, this results in superior antitumor responses with cellular therapy.

While prior methods propose inhibition of glycolysis as a means to improve T cell therapy, this has serious adverse effects on expansion and generation of therapeutic cells. In contrast, the inventors propose that redirection, rather than inhibition, of glucose flux in cells leads to superior therapeutic efficacy in a way that does not impact the generation of these cells. Thus, PDHK1, PGC1α, and PDP pathways can be targeted pharmacologically (small molecules) or genetically (transient expression of PDHK1 inhibitors, PGC1α peptides and activators, or PDP activators) to improve therapeutic T cell function and fate. Specifically, the methods herein can include culturing immune effector cells (e.g., T cells) in the presence of one or more metabolic enhancers selected from the group consisting of pyruvate dehydrogenase kinase 1 (PDHK1) inhibitors, pyruvate dehydrogenase phosphatase (PDP) activators, peroxisome proliferator-activated receptor gamma coactivator 1-alpha (PGC1α) polypeptides or variants thereof, and PGC1α agonists, after the immune effector cells have been activated and transfected with a vector (such as a viral vector) including a polynucleotide encoding an engineered antigen receptor (e.g., engineered T cell receptor (TCR), dimerizing agent regulated immunoreceptor complex (DARIC), or chimeric antigen receptor (CAR)).

The disclosure generally relates to improved methods for manufacturing immune effector cells (e.g., T cells). Without wishing to be bound to any particular theory, the methods contemplated herein culture immune effector cells (e.g., T cells) in the presence of one or more metabolic enhancers selected from the group consisting of PDHK1 inhibitors/antagonists, PDP activators/agonists, PGC1α polypeptides or variants thereof, and PGC1α agonists, after the immune effector cells (e.g., T cells) have been activated, stimulated, and transfected with a vector (such as a viral vector) comprising a polynucleotide encoding an engineered antigen receptor (e.g., TCR, CAR, or DARIC) to produce immune effector cells (e.g., T cells) having superior properties (e.g., increased respiratory capacity and/or increased mitochondrial mass), compared to existing immune effector cell (e.g., T cell) compositions. Accordingly, immune effector cell (e.g., T cell) compositions contemplated herein include immune effector cells (e.g., T cells), which in some examples, have increased survival, expansion, and persistence in vivo. As a result, methods using such immune effector cell (e.g., T cell) compositions can have superior results, such as a superior ability to treat cancer, infectious disease, autoimmune disease, inflammatory disease, or immunodeficiency, for example as compared to an immune effector cell (e.g., T cell) composition not cultured in the presence of one or more metabolic enhancers selected from the group consisting of PDHK1 inhibitors/antagonists, PDP activators/agonists, PGC1α polypeptides or variants thereof, and PGC1α agonists.

Manufactured immune effector cell (e.g., T cell) compositions contemplated herein are useful in the treatment or prevention of numerous conditions including, but not limited to cancer, infectious disease, autoimmune disease, inflammatory disease, and immunodeficiency. Without wishing to be bound to any particular theory, the inventors haves surprisingly and unexpectedly discovered that redirecting glucose towards oxidation (e.g., antagonizes glycolysis promoting signature during expansion/culturing step) into the mitochondria and away from lactose fermentation in immune effector cells (e.g., T cells) during the culturing stage (e.g., after activation, stimulation, and transformation with a vector (such as a viral vector) comprising a polynucleotide encoding an engineered antigen receptor (e.g., TCR, CAR, or DARIC)), results in substantial improvement in respiratory capacity of the immune effector cells (e.g., T cells) and superior results on cancer treatment in vivo, compared to immune effector cells (e.g., T cells) where glucose is not redirected.

Accordingly, the methods and compositions contemplated herein represent a quantum improvement compared to existing adoptive cell immunotherapies.

### III. Methods of Manufacturing Immune Effector Cells

The immune effector cells (e.g., T cells) manufactured by the methods provided herein provide improved adoptive immunotherapy compositions. Without being bound to any particular theory, it is believed that the immune effector cells (e.g., T cells) manufactured by the methods contemplated herein are imbued with superior properties, including increased respiratory capacity and/or increased mitochondrial mass. In some examples, the respiratory capacity of the transduced immune effector cells (e.g., T cells) cultured in the presence of one or more metabolic enhancers selected from the group consisting of: PDHK1 inhibitors/antagonists, PDP activators/agonists, PGC1α polypeptides or variants thereof, and PGC1α agonists, is increased by at least 10%, at least 20%, at least 30%, at least 40%, at least 50%, at least 75%, at least 90%, at least 2-fold, or at least 3-fold, compared to transduced immune effector cells (e.g., T cells) cultured in the absence of one or more metabolic enhancers selected from the group consisting of PDHK1 inhibitors/antagonists, PDP activators/agonists, PGC1α polypeptides or variants thereof, and PGC1α agonists. In some examples, the mitochondrial mass of the transduced immune effector cells (e.g., T cells) cultured in the presence of one or more metabolic enhancers selected from the group consisting of: PDHK1 inhibitors/antagonists, PDP activators/agonists, PGC1α polypeptides or variants thereof, and PGC1α agonists, is increased by at least 20%, at least 30%, at least 40%, or at least 50%, compared to transduced immune effector cells (e.g., T cells) cultured in the absence of one or more metabolic enhancers selected from the group consisting of: PDHK1 inhibitors/antagonists, PDP activators/agonists, PGC1α polypeptides or variants thereof, and PGC1α agonists. In some examples, combinations of these effects are achieved.

In one embodiment, a method of manufacturing immune effector cells comprises (a) activating a population of immune effector cells; (b) transducing the immune effector cells with a vector comprising a polynucleotide encoding an engineered antigen receptor; and (c) culturing the transduced immune effector cells to proliferate; wherein any one or more of steps (a)-(c) is performed in the presence of one or more metabolic enhancers selected from the group consisting of: PDHK1 inhibitors/antagonists, PDP activators/agonists, PGC1α polypeptides or variants thereof, and PGC1α agonists. Such a method can increase the respiratory capacity of the transduced immune effector cells by at least 10% relative to transduced immune effector cells cultured in the absence of the one or more of the metabolic enhancers

Provided herein are methods for increasing respiratory capacity of immune effector cells. Such methods can include (a) activating a population of immune effector cells; (b) transducing the immune effector cells with a vector comprising a polynucleotide encoding an engineered antigen receptor; and (c) culturing the transduced immune effector cells to proliferate, wherein any one or more of steps (a)-(c) is performed in the presence of one or more metabolic enhancers selected from the group consisting of PDHK1 inhibitors, PDP activators, PGC1α polypeptides or variants thereof, and PGC1α agonists. Such a method can increase the respiratory capacity of the transduced immune effector cells is increased by at least 10% relative to transduced immune effector cells cultured in the absence of the one or more of the metabolic enhancers.

Provided herein are methods for increasing the mitochondrial mass of immune effector cells. Such methods can include (a) activating a population of immune effector cells; (b) transducing the immune effector cells with a vector comprising a polynucleotide encoding an engineered antigen receptor; and (c) culturing the transduced immune effector cells to proliferate; wherein any one or more of steps (a)-(c) is performed in the presence of one or more metabolic enhancers selected from the group consisting of PDHK1 inhibitors, PDP activators, PGC1α polypeptides or variants thereof, and PGC1α agonists. Such a method can increase the mitochondrial mass of the transduced immune effector cells by at least 25%, at least 30%, at least 40%, or at least 50%, relative to transduced immune effector cells cultured in the absence of the one or more of the metabolic enhancers.

In some embodiments, the methods of manufacturing immune effector cells, increasing respiratory capacity of immune effector cells, or methods for increasing the mitochondrial mass of immune effector cells, comprises (i) performing one or more of steps (a)-(c) in the presence of DCA and (ii) electroporating the immune effector cells with an mRNA encoding a biologically active PGC1α polypeptide fragment selected from the group consisting of amino acids 1-270 of PGC1α (e.g., aa 1-270 of SEQ ID NO: 18 or 19), amino acids 1-290 of PGC1α (e.g., aa 1-290 of SEQ ID NO: 18 or 19), or no more than about the first (N-terminal) 270 or 290 amino acids of PGC1α (e.g., no more than about the first (N-terminal) 270 or 290 aa of SEQ ID NO: 18 or 19).

In some embodiments, the methods of manufacturing immune effector cells, increasing respiratory capacity of immune effector cells, or methods for increasing the mitochondrial mass of immune effector cells, comprises (i) performing one or more of steps (a)-(c) in the presence of DCA and (ii) transducing the immune effector cells with a vector comprising a polynucleotide encoding an engineered antigen receptor and a polynucleotide encoding a biologically active PGC1α polypeptide fragment selected from the group consisting of amino acids 1-270 of PGC1α (e.g., aa 1-270 of SEQ ID NO: 18 or 19), amino acids 1-290 of PGC1α (e.g., aa 1-290 of SEQ ID NO: 18 or 19), or no more than about the first (N-terminal) 270 or 290 amino acids of PGC1α (e.g., no more than about the first (N-terminal) 270 or 290 aa of SEQ ID NO: 18 or 19).

Examples of immune effector cells that can be used with the disclosed methods include but are not limited to: T cells, αβ-T cells, γδ-T cells, natural killer (NK) cells, natural killer T (NKT) cells, CD4+ T cells, and CD8+ T cells. In some examples, the immune effector cells are a purified population of T cells, αβ-T cells, γδ-T cells, natural killer (NK) cells, natural killer T (NKT) cells, CD4+ T cells, or CD8+ T cells, such as a purified population of CD4+ T cells and/or CD8+ T cells.

In some examples, the method also includes isolating a population of immune effector cells (such as T cells, e.g., tumor infiltrating cytotoxic T lymphocytes (TIL)), from a subject, prior to the activating step (a), for example isolating peripheral blood mononuclear cells (PBMCs) as the source of immune effector cells.

In some examples, activating a population of immune effector cells comprises stimulating the immune effector cells to proliferate in the presence of (i) interleukin 2 (IL-2); (ii) an anti-CD3 antibody or antigen binding fragment thereof, and (iii) an anti-CD28 antibody or antigen binding fragment thereof.

In some examples, the immune effector cells are transduced with a viral vector. In some embodiments, the immune effector cells are transduced with the vector prior to their activation. In certain embodiments, the immune effector cells are transduced with the vector after being activated. In particular embodiments, the viral vector is a retroviral vector. In some embodiments, the viral vector is a lentiviral vector.

In some embodiments, one or more of the metabolic enhancers is introduced into the immune effector cells by mRNA electroporation or by lipid nanoparticle. In some examples, the vector comprising a polynucleotide encoding an engineered antigen receptor further comprises a polynucleotide encoding one or more of the metabolic enhancers.

In some examples, prior to culturing, the immune effector cells can be modified to express one or more engineered antigen receptors, such as TCRs, DARICs, or CARs. In one embodiment, the immune effector cells are modified by transducing the immune effector cells with a viral vector comprising an engineered TCR, DARIC, or CAR. In a certain embodiment, the immune effector cells are modified prior to stimulation and activation. In another embodiment, immune effector cells are modified after stimulation and activation. In a particular embodiment, immune effector cells are modified within 12 hours, 24 hours, 36 hours, or 48 hours of stimulation and activation.

In some examples, after the immune effector cells are activated, stimulated, and transformed, the cells are cultured to proliferate in the presence of one or more metabolic enhancers selected from the group consisting of: PDHK1 inhibitors/antagonists, PDP activators/agonists, PGC1α polypeptides or variants thereof, and PGC1α agonists. Immune effector cells may be cultured for at least 5 days, such as at least 6, at least 7, at least 8, at least 9, or at least 10 days. In some examples, immune effector cells may be cultured for at least 2 weeks, at least 1, 2, 3, 4, 5, or 6 months or more with at least 1, at least 2, at least 5 rounds of expansion, such as 1, 2, 3, 4, 5, 6, 7, 8, 9, or 10 or more rounds of expansion.

In particular embodiments, the one or more inhibitors of PDHK1 comprise a peptide, a PDHK1 antibody or antibody fragment, a small molecule, or a combination of two or more thereof. In particular embodiments, the small molecule is dichloroacetic acid (DCA). In particular embodiments, the small molecule is AZD7545. In particular embodiments, the one or more inhibitors of PDHK1 comprise a PDHK1 inhibitory RNA (RNAi) molecule, a PDHK1 aptamer, or combinations thereof. In particular embodiments, the PDHK1 RNAi molecule is a PDHK1 antisense molecule, a PDHK1 siRNA molecule, a PDHK1 microRNA molecule, or combinations thereof.

In particular embodiments, the PDP activator comprises a peptide, an enzyme, a PDP antibody, a PDP antibody fragment, a small molecule, or a combination of two or more thereof. In particular embodiments, the peptide comprises insulin. In particular embodiments, the small molecule comprises PEP, AMP, or a combination thereof. In particular embodiments, the PDP activator comprises a PDP protein, PDP coding sequence, PDP antibody or antibody fragment, or a combination thereof.

In particular embodiments, the PGC1α polypeptide or variant thereof is a full length PGC1α polypeptide (e.g., SEQ ID NO: 18 or 19), a PGC1α polypeptide comprising one or more amino acid insertions, deletions, or substitutions, or a biologically active PGC1α polypeptide fragment. In particular embodiments, the PGC1α polypeptide or variant thereof is a biologically active PGC1α polypeptide fragment. In one example, the metabolic enhancer includes a PGC1α polypeptide or variant thereof, such as one comprising at least 80%, at least 90%, at least 95%, at least 98%, at least 99% or 100% sequence identity to SEQ ID NO: 18 or 19, while retaining PGC1α activity. In one example, the PGC1α polypeptide or variant thereof is a biologically active PGC1α polypeptide fragment comprising or consisting of amino acids 1-270 of PGC1α, amino acids 1-290 of PGC1α, or no more than about the first (N-terminal) 270 or 290 amino acids of PGC1α, such as aa 1-270 of SEQ ID NO: 18 or 19, aa 1-290 of SEQ ID NO; 18 or 19, such as one comprising at least 80%, at least 90%, at least 95%, at least 98%, at least 99% or 100% sequence identity to aa 1-270 of SEQ ID NO: 18 or 19, or one comprising at least 80%, at least 90%, at least 95%, at least 98%, at least 99% or 100% sequence identity to aa 1-290 of SEQ ID NO: 18 or 19, while retaining PGC1α activity. In one example the PGC1α activator is

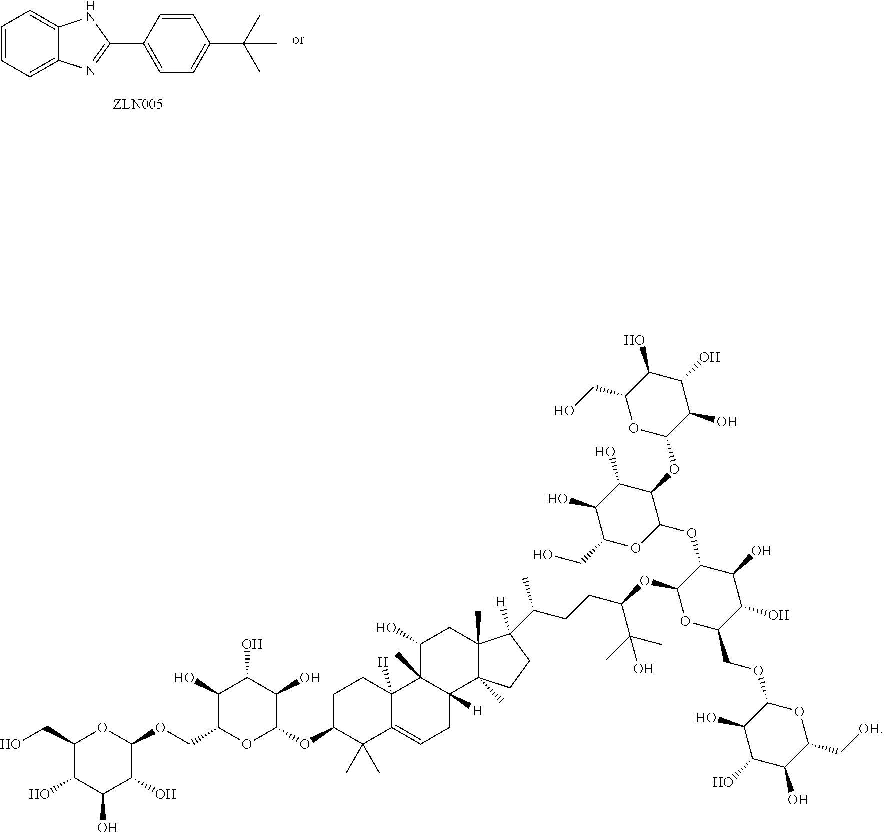 [SMILES: CC(C)(C)c1ccc(-c2nc3ccccc3[nH]2)cc1 ; C[C@H](CC[C@@H](O[C@@H]1O[C@H](CO[C@@H]2O[C@H](CO)[C@@H](O)[C@H](O)[C@H]2O)[C@@H](O)[C@H](O)[C@H]1OC1O[C@H](CO)[C@@H](O)[C@H](O)[C@H]1O[C@@H]1O[C@H](CO)[C@@H](O)[C@H](O)[C@H]1O)C(C)(C)O)[C@H]1CC[C@@]2(C)[C@@H]3CC=C4[C@@H](CC[C@H](O[C@@H]5O[C@H](CO[C@@H]6O[C@H](CO)[C@@H](O)[C@H](O)[C@H]6O)[C@@H](O)[C@H](O)[C@H]5O)C4(C)C)[C@]3(C)[C@H](O)C[C@]12C]

In one embodiment, peripheral blood mononuclear cells (PBMCs) are used as a source of immune effector cells (e.g., T cells) in the manufacturing methods contemplated herein. PBMCs form a heterogeneous population of T lymphocytes that can be CD4³⁰, CD8⁺, or CD4⁺ and CD8⁺ and can include other mononuclear cells such as monocytes, B cells, NK cells and NKT cells. An expression vector comprising a polynucleotide encoding an engineered TCR or CAR contemplated herein can be introduced into a population of human donor T cells, NK cells or NKT cells. Successfully transduced T cells that carry the expression vector can be sorted using flow cytometry to isolate CD3 positive T cells and then further propagated to increase the number of the modified T cells in addition to cell activation using anti-CD3 antibodies and or anti-CD28 antibodies and IL-2, IL-7, and/or IL-15 or any other methods known in the art as described elsewhere herein.

Manufacturing methods contemplated herein may further comprise cryopreservation of modified immune effector cells (e.g., T cells) for storage and/or preparation for use in a mammalian subject, such as a human subject. Immune effector cells (e.g., T cells) are cryopreserved such that the cells remain viable upon thawing. When needed, the cryopreserved transformed immune effector cells can be thawed, grown and expanded for more such cells. As used herein, “cryopreserving,” refers to the preservation of cells by cooling to sub-zero temperatures, such as (typically) 77 K or −196 °C (the boiling point of liquid nitrogen). Cryoprotective agents are often used at sub-zero temperatures to prevent the cells being preserved from damage due to freezing at low temperatures or warming to room temperature. Cryopreservative agents and optimal cooling rates can protect against cell injury. Cryoprotective agents which can be used include but are not limited to dimethyl sulfoxide (DMSO) (Lovelock and Bishop, Nature, 1959; 183: 1394-1395; Ashwood-Smith, Nature, 1961; 190: 1204-1205), glycerol, polyvinylpyrrolidine (Rinfret, Ann. N.Y. Acad. Sci., 1960; 85: 576), and polyethylene glycol (Sloviter and Ravdin, Nature, 1962; 196: 48). In one example, the cooling rate is 1° to 3 °C/minute. After at least two hours, the T cells have reached a temperature of −80 °C and can be placed directly into liquid nitrogen (−196 °C) for permanent storage such as in a long-term cryogenic storage vessel. Thus, provided herein is a population of immune effector cells (e.g., T cells) generated using the disclosed methods, and a cryopreservation agent, such as DMSO or polyethylene glycol.

### A. Immune Effector Cells

Provided are methods of manufacturing improved immune effector cells (e.g., T cells), such as those with increased respiratory capacity and/or increased mitochondrial mass, and compositions containing such. Immune effector cells (e.g., T cells) may be autologous/autogeneic (“self”) or non-autologous (“non-self,” e.g., allogeneic, syngeneic or xenogeneic). In some embodiments, the immune effector cells (e.g., T cells) are obtained from a mammalian subject. In some embodiments, the immune effector cells (e.g., T cells) are obtained from a primate subject. In one embodiment, the immune effector cells (e.g., T cells) are obtained from a human subject.

Immune effector cells (e.g., T cells) can be obtained from a number of sources including, but not limited to, peripheral blood mononuclear cells, bone marrow, lymph nodes tissue, cord blood, thymus issue, tissue from a site of infection, ascites, pleural effusion, spleen tissue, and tumors. In certain embodiments, immune effector cells (e.g., T cells) are obtained from a unit of blood collected from a subject using any number of techniques, such as sedimentation, e.g., FICOLL™ separation. Thus, in some examples, the disclosed methods include obtaining immune effector cells (e.g., T cells) from a subject, prior to their activation and stimulation. In one embodiment, cells from the circulating blood of an individual are obtained by apheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing. The cells can be washed with PBS or with another suitable solution that lacks calcium, magnesium, and most, if not all other, divalent cations. A washing step may be accomplished by methods known to those in the art, such as by using a semiautomated flowthrough centrifuge. For example, the Cobe 2991 cell processor, the Baxter CytoMate, or the like. After washing, the cells may be resuspended in a variety of biocompatible buffers or other saline solution with or without buffer. In certain embodiments, the undesirable components of the apheresis sample may be removed in the cell directly resuspended culture media.

In particular embodiments, a population of cells comprising immune effector cells (e.g., T cells), e.g., PBMCs, is used in the manufacturing methods contemplated herein. In other embodiments, an isolated or purified population of immune effector cells (e.g., T cells) is used in the manufacturing methods contemplated herein. Cells can be isolated from peripheral blood mononuclear cells (PBMCs) by lysing the red blood cells and depleting the monocytes, for example, by centrifugation through a PERCOLL® gradient. In some embodiments, after isolation of PBMC, both cytotoxic and helper T lymphocytes can be sorted into naïve, memory, and effector T cell subpopulations either before or after activation, expansion, and/or genetic modification.

A specific subpopulation of T cells, expressing one or more of the following markers: CD3, CD4, CD8, CD28, CD45RA, CD45RO, CD62, CD127, and HLA-DR can be further isolated by positive or negative selection techniques. In one embodiment, a specific subpopulation of T cells, expressing one or more of the markers selected from the group consisting of CD62L, CCR7, CD28, CD27, CD122, CD127, CD197; or CD38 or CD62L, CD127, CD197, and CD38, is further isolated by positive or negative selection techniques. In various embodiments, the manufactured T cell compositions do not express or do not substantially express one or more of the following markers: CD57, CD244, CD160, PD-1, CTLA4, TIM3, and LAG3.

With respect to T cells, the T cell populations resulting from the various expansion methodologies contemplated herein may have a variety of specific phenotypic properties, depending on the conditions employed. In various embodiments, expanded T cell populations comprise one or more of the following phenotypic markers: CD62L, CD127, CD197, CD38, and HLA-DR.

In one embodiment, such phenotypic markers include enhanced expression of one or more of, or all of CD62L, CD127, CD197, and CD38. In particular embodiments, CD8⁺ T lymphocytes characterized by the expression of phenotypic markers of naive T cells including CD62L, CD127, CD197, and CD38 are expanded.

In particular embodiments, T cells characterized by the expression of phenotypic markers of central memory T cells including CD45RO, CD62L, CD127, CD197, and CD38 and negative for granzyme B are expanded. In some embodiments, the central memory T cells are CD45RO⁺, CD62L⁺, CD8⁺ T cells. In certain embodiments, CD4⁺ T lymphocytes characterized by the expression of phenotypic markers of naïve CD4⁺ cells including CD62L and negative for expression of CD45RA and/or CD45RO are expanded. In some embodiments, CD4⁺ cells characterized by the expression of phenotypic markers of central memory CD4⁺ cells including CD62L and CD45RO positive. In some embodiments, effector CD4⁺ cells are CD62L positive and CD45RO negative.

In certain embodiments, the immune effector cells (e.g., T cells) are isolated from an individual and modified without further manipulation ex vivo or in vitro. Such cells can then be directly re-administered into the individual. In further embodiments, the immune effector cells (e.g., T cells) are first activated and stimulated to proliferate in vitro prior to being genetically modified to express an engineered TCR, DARIC, or CAR. In this regard, the immune effector cells (e.g., T cells) may be cultured before and/or after being genetically modified (i.e., transduced or transfected to express an engineered TCR, DARIC, or CAR contemplated herein).

### B. Activation, Stimulation, and Culturing

In order to achieve sufficient therapeutic doses of immune effector cells (e.g., T cells) compositions, immune effector cells (e.g., T cells) are often subjected to one or more rounds of stimulation, activation and expansion (culturing). T cells and other immune effector cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041, each of which is incorporated herein by reference in its entirety. T cells or other immune effector cells modified to express an engineered antigen receptor, such as TCR, DARIC, or CAR, can be activated and stimulated before and/or after the immune effector cells are modified. In addition, T cells or other immune effector cells may be contacted with one or more metabolic enhancers selected from the group consisting of: PDHK1 inhibitors, PDP activators, PGC1α polypeptides or variants thereof, and PGC1α agonists, after activation, stimulation, and transformation. In one embodiment, T cells or other immune effector cells manufactured by the methods contemplated herein undergo one, two, three, four, or five or more rounds of activation, stimulation, and culturing.

In one embodiment, a costimulatory ligand is presented on an antigen presenting cell (e.g., an APC, dendritic cell, B cell, and the like) that specifically binds a cognate costimulatory molecule on a T cell or other immune effector cell, thereby providing a signal which, in addition to the primary signal provided by, for instance, binding of a TCR/CD3 complex, mediates a desired T cell response. Suitable costimulatory ligands include, but are not limited to, CD7, B7-1 (CD80), B7-2 (CD86), PD-L 1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, ILT3, ILT4, an agonist or antibody that binds Toll ligand receptor, and a ligand that specifically binds with B7-H3.

In a particular embodiment, a costimulatory ligand comprises an antibody or antigen binding fragment thereof that specifically binds to a costimulatory molecule present on a T cell or other immune effector cells, including but not limited to, CD27, CD28, 4-IBB, OX40, CD30, CD40, PD-1, 1COS, lymphocyte function-associated antigen-1 (LFA-1), CD7, LIGHT, NKG2C, B7-H3, and a ligand that specifically binds with CD83.

Suitable costimulatory ligands further include target antigens, which may be provided in soluble form or expressed on APCs or aAPCs that bind engineered TCRs, DARICs, or CARs expressed on modified immune effector cells.

In various embodiments, a method for manufacturing immune effector cells contemplated herein comprises activating a population of cells comprising immune effector cells and expanding the population of immune effector cells. Immune effector cell (e.g., T cell) activation can be accomplished by providing a primary stimulation signal through the TCR/CD3 complex or via stimulation of the CD2 surface protein and by providing a secondary costimulation signal through an accessory molecule, e.g., CD28.

The TCR/CD3 complex may be stimulated by contacting the cell with a suitable CD3 binding agent, e.g., a CD3 ligand or an anti-CD3 monoclonal antibody. Illustrative examples of CD3 antibodies include, but are not limited to, OKT3, G19-4, BC3, and 64.1.

In another embodiment, a CD2 binding agent may be used to provide a primary stimulation signal to the cells. Illustrative examples of CD2 binding agents include, but are not limited to, CD2 ligands and anti-CD2 antibodies, e.g., the T11.3 antibody in combination with the T11.1 or T11.2 antibody (Meuer, S. C. et al. (1984) Cell 36:897-906) and the 9.6 antibody (which recognizes the same epitope as TI 1.1) in combination with the 9-1 antibody (Yang, S. Y. et al. (1986) J. Immunol. 137:1097-1100). Other antibodies which bind to the same epitopes as any of the above described antibodies can also be used. Additional antibodies, or combinations of antibodies, can be prepared and identified by standard techniques as disclosed elsewhere herein.

In addition to the primary stimulation signal provided through the TCR/CD3 complex, or via CD2, induction of immune effector cell (e.g., T cell) responses requires a second, costimulatory signal. In particular embodiments, a CD28 binding agent can be used to provide a costimulatory signal. Illustrative examples of CD28 binding agents include but are not limited to: natural CD28 ligands, e.g., a natural ligand for CD28 (e.g., a member of the B7 family of proteins, such as B7-1(CD80) and B7-2 (CD86); and anti-CD28 monoclonal antibody or fragment thereof capable of crosslinking the CD28 molecule, e.g., monoclonal antibodies 9.3, B-T3, XR-CD28, KOLT-2, 15E8, 248.23.2, and EX5.3D10.

In one embodiment, the molecule providing the primary stimulation signal, for example a molecule which provides stimulation through the TCR/CD3 complex or CD2, and the costimulatory molecule are coupled to the same surface.

In certain embodiments, binding agents that provide stimulatory and costimulatory signals are localized on the surface of a cell. This can be accomplished by transfecting or transducing a cell with a nucleic acid encoding the binding agent in a form suitable for its expression on the cell surface or alternatively by coupling a binding agent to the cell surface.

In another embodiment, the molecule providing the primary stimulation signal, for example a molecule which provides stimulation through the TCR/CD3 complex or CD2, and the costimulatory molecule are displayed on antigen presenting cells.

In one embodiment, the molecule providing the primary stimulation signal, for example a molecule which provides stimulation through the TCR/CD3 complex or CD2, and the costimulatory molecule are provided on separate surfaces.

In a certain embodiment, one of the binding agents that provide stimulatory and costimulatory signals is soluble (provided in solution) and the other agent(s) is provided on one or more surfaces.

In a particular embodiment, the binding agents that provide stimulatory and costimulatory signals are both provided in a soluble form (provided in solution).

In various embodiments, the methods for manufacturing immune effector cells (e.g., T cells) contemplated herein comprise activating immune effector cells (e.g., T cells) with anti-CD3 and anti-CD28 antibodies.

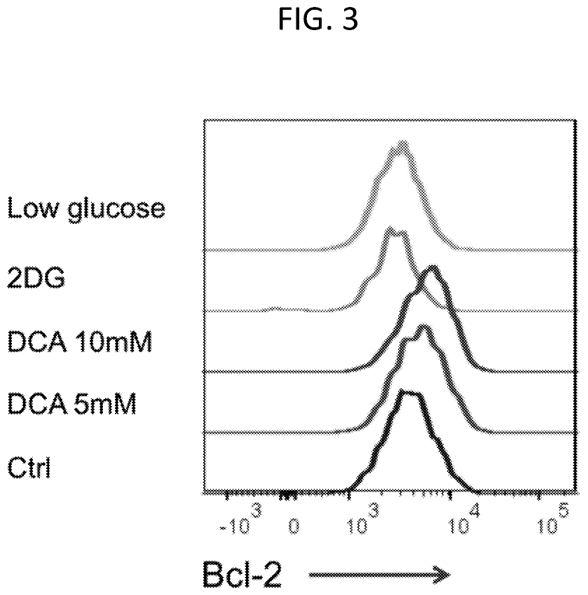

Immune effector cell (e.g., T cell) compositions manufactured by the methods contemplated herein comprise transformed immune effector cells (e.g., T cells) expanded/cultured in the presence of one or more metabolic enhancers selected from the group consisting of PDHK1 inhibitors, PDP activators, PGC1α polypeptides or variants thereof, and PGC1α agonists (FIG. 3). T cells modified to express an engineered antigen receptor, such as an engineered TCR, DARIC, or CAR can be activated/stimulated before and/or after the immune effector cells (e.g., T cells) are transformed. In particular embodiments, a population of immune effector cells (e.g., T cells) is activated, modified to express an engineered TCR, DARIC, or CAR, and then cultured for expansion.

In one embodiment, immune effector cells (e.g., T cells) manufactured by the methods contemplated herein comprise increased respiratory capacity by at least 10%, at least 20%, at least 30%, at least 40%, at least 50%, at least 75%, at least 90%, at least 2-fold, or at least 3-fold, compared to transduced immune effector cells (e.g., T cells) cultured in the absence of one or more metabolic enhancers selected from the group consisting of PDHK1 inhibitors, PDP activators, PGC1α polypeptides or variants thereof, and PGC1α agonists. In one embodiment, immune effector cells (e.g., T cells) manufactured by the methods contemplated herein have an increased mitochondrial mass of at least 10%, at least 20%, at least 30%, at least 40%, or at least 50%, compared to transduced immune effector cells (e.g., T cells) cultured in the absence of one or more metabolic enhancers selected from the group consisting of PDHK1 inhibitors, PDP activators, PGC1α polypeptides or variants thereof, and PGC1α agonists. These immune effector cells (e.g., T cells) provide an improved therapeutic immune effector cell (e.g., T cell) composition. For example, the respiratory capacity of the transduced immune effector cells (e.g., T cells) can be increased when one or more metabolic enhancers selected from the group consisting of PDHK1 inhibitors, PDP activators, PGC1α polypeptides or variants thereof, and PGC1α agonists, redirects metabolism of glucose toward oxidation (e.g., toward the mitochondria and away from fermentation into lactate) in the population of transduced immune effector cells (e.g., T cells).

In one embodiment, culturing/expanding the transformed immune effector cells (e.g., T cells) by the methods includes culturing the transformed immune effector cells (e.g., T cells) for at least 5 days, at least 6 days, at least 7 days, at least 8 days, at least 9 days, or at least 10 days, for example 5 to 10 days, 5 to 28 days, or 7 to 28 days. In another embodiment, the immune effector cell (e.g., T cell) composition is cultured for 14 days. In a particular embodiment, immune effector cells (e.g., T cells) are cultured for about 21 days. In another embodiment, the immune effector cell (e.g., T cell) compositions are cultured for about 2-3 days. Several cycles of stimulation/activation/expansion may also be desired such that culture time of immune effector cells (e.g., T cells) can be at least 30 days, at least 45 days, at least 60 days, or more.

In particular embodiments, conditions appropriate for immune effector cell (e.g., T cell) culture include an appropriate media (e.g., Minimal Essential Media or RPMI Media 1640 or, X-Vivo®-15, (Lonza)) and one or more factors necessary for proliferation and viability including, but not limited to serum (e.g., fetal bovine or human serum), interleukin-2 (IL-2), insulin, IFN-γ, IL-4, IL-7, IL-21, GM-CSF, IL-10, IL-12, IL-15, TGFβ, and TNF-α or any other additives suitable for the growth of cells.

Further illustrative examples of cell culture media include, but are not limited to RPMI 1640, Clicks, AIM-V, DMEM, MEM, a-MEM, F-12, X-Vivo®-1 5, and X-Vivo®20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells.

Illustrative examples of other additives for immune effector cell (e.g., T cell) expansion include, but are not limited to, surfactant, piasmanate, pH buffers such as HEPES, and reducing agents such as N-acetyl-cysteine and 2-mercaptoethanol

Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37 °C) and atmosphere (e.g., air plus 5% C02).

In particular embodiments, PBMCs or isolated immune effector cells (e.g., T cells) are contacted with a stimulatory agent and costimulatory agent, such as anti-CD3 and anti-CD28 antibodies, generally attached to a bead or other surface, in a culture medium with appropriate cytokines, such as IL-2, IL-7, and/or IL-15.

In other embodiments, artificial APC (aAPC) made by engineering K562, U937, 721.221, T2, and C1R cells to direct the stable expression and secretion, of a variety of costimulatory molecules and cytokines. In a particular embodiment K32 or U32 aAPCs are used to direct the display of one or more antibody-based stimulatory molecules on the AAPC cell surface. Populations of T cells or other immune effector cells can be expanded by aAPCs expressing a variety of costimulatory molecules including, but not limited to, CD137L (4-1BBL), CD134L (OX40L), and/or CD80 or CD86. Finally, the aAPCs provide an efficient platform to expand genetically modified T cells and to maintain CD28 expression on CD8 T cells. aAPCs provided in WO 03/057171 and US2003/0147869 are hereby incorporated by reference in their entirety.

### 1. Agents

In various embodiments, a method for manufacturing immune effector cells (e.g., T cells) is provided that cultures activated, stimulated and transformed immune effector cells (e.g., T cells) comprising contacting immune effector cells (e.g., T cells) with one or more metabolic enhancers selected from the group consisting of PDHK1 inhibitors, PDP activators, PGC1α polypeptides or variants thereof, and PGC1α agonists. In various embodiments, a method for manufacturing immune effector cells (e.g., T cells) with increased respiratory capacity is provided that cultures activated and transformed immune effector cells (e.g., T cells) including contacting immune effector cells (e.g., T cells) with one or more metabolic enhancers selected from the group consisting of PDHK1 inhibitors, PDP activators, PGC1α polypeptides or variants thereof, and PGC1α agonists. The immune effector cell (e.g., T cell) compositions retain sufficient immune effector cell (e.g., T cell) potency such that they may undergo multiple rounds of expansion without a substantial increase in differentiation.

As used herein, the terms “modulate,” “modulator,” or “modulatory agent” or comparable term refer to an agent's ability to elicit a change in a cell signaling pathway. A modulator may increase or decrease an amount, activity of a pathway component or increase or decrease a desired effect or output of a cell signaling pathway. In one embodiment, the modulator is an inhibitor. In another embodiment, the modulator is an activator.

An “agent” refers to a compound, small molecule, e.g., small organic molecule, nucleic acid molecule, peptide, antibody or antibody fragment, aptamer, isoform, variant, analog, or derivative thereof used in the inhibition of PDHK1, activation of PGC1α, or activation of PDP.

A “small molecule” refers to a composition that has a molecular weight of less than about 5 kD, less than about 4 kD, less than about 3 kD, less than about 2 kD, less than about 1 kD, or less than about 0.5 kD. Small molecules may comprise nucleic acids, peptides, polypeptides, peptidomimetics, peptoids, carbohydrates, lipids, components thereof or other organic or inorganic molecules. Libraries of chemical and/or biological mixtures, such as fungal, bacterial, or algal extracts, are known and can be screened with any of the assays of the disclosure. Examples of methods for the synthesis of molecular libraries can be found in: (Carell et al., 1994a; Carell et al., 1994b; Cho et al., 1993; DeWitt et al., 1993; Gallop et al., 1994; Zuckermann et al., 1994).

An “analog” refers to a small organic compound, a nucleotide, a protein, or a polypeptide that possesses similar or identical activity or function(s) as the compound, nucleotide, protein or polypeptide or compound having the desired activity of the present disclosure, but need not necessarily comprise a sequence or structure that is similar or identical to the sequence or structure of the preferred embodiment.

A “derivative” refers to either a compound, a protein or polypeptide that comprises an amino acid sequence of a parent protein or polypeptide that has been altered by the introduction of amino acid residue substitutions, deletions or additions, or a nucleic acid or nucleotide that has been modified by either introduction of nucleotide substitutions or deletions, additions or mutations. The derivative nucleic acid, nucleotide, protein or polypeptide possesses a similar or identical function as the parent polypeptide.

In various embodiments, an agent activates PDP or PGC1α activity. An “activator,” or “agonist” refers to an agent that promotes, increases, or induces one or more activities of PDP or PGC1α. In some examples, such an agent increases PDP activity in a T cell by at least 10%, at least 20%, at least 30%, at least 40%, at least 50%, at least 75%, at last 80%, at least 90%, at least 95%, at least 100%, at least 200%, at least 300%, at least 400%, or at least 500%.

In various embodiments, an agent inhibits PDHK1. An “inhibitor” or “antagonist” refers to an agent that inhibits, decreases, or reduces one or more activities of PDHK1. In one embodiment, the inhibitor is a dual molecule inhibitor. In particular embodiment, the inhibitor may inhibit a class of molecules have the same or substantially similar activities (a pan-inhibitor) or may specifically inhibit a molecule's activity (a selective or specific inhibitor). Inhibition may also be irreversible or reversible. Inhibition of PDHK1 activity need not be 100%. In some examples, an agent inhibits or reduces PDP activity in a T cell by at least 10%, at least 20%, at least 30%, at least 40%, at least 50%, at least 75%, at last 80%, at least 90%, at least 95%, at least 98% at least 99%, or 100%.

In one embodiment, the PDHK1 inhibitor has an IC50 of at least 1 nM, at least 2 nM, at least 5 nM, at least 10 nM, at least 50 nM, at least 100 nM, at least 200 nM, at least 500 nM, at least 1 μM, at least 10 μM, at least 50 μM, or at least 100 μM. IC50 determinations can be accomplished using any conventional techniques. For example, an IC50 can be determined by measuring the activity of PDHK1 in the presence of a range of concentrations of the inhibitor under study. The experimentally obtained values of enzyme activity then are plotted against the inhibitor concentrations used. The concentration of the inhibitor that shows 50% PDHK1 activity (as compared to the activity in the absence of any inhibitor) is taken as the “IC50” value. Analogously, other inhibitory concentrations can be defined through appropriate determinations of activity.

In various embodiments, T cells are contacted or treated or cultured with one or more PDHK1 inhibitors at a concentration of at least 1 nM, at least 2 nM, at least 5 nM, at least 10 nM, at least 50 nM, at least 100 nM, at least 200 nM, at least 500 nM, at least 1 μM, at least 10 μM, at least 50 M, at least 100 μM, or at least 1 M, such as 1 to 100 mM, 1 to 20 mM, or 5 to 10 mM. In various embodiments, T cells are contacted or treated or cultured with one or more PDP activators at a concentration of at least 1 nM, at least 2 nM, at least 5 nM, at least 10 nM, at least 50 nM, at least 100 nM, at least 200 nM, at least 500 nM, at least 1 μM, at least 10 μM, at least 50 μM, at least 100 μM, or at least 1 M, such as 1 to 100 mM, 1 to 20 mM, or 5 to 10 mM.

In particular embodiments, activated, stimulated, transduced T cells are contacted or treated or cultured with one or more metabolic enhancers, such as PDHK1 inhibitors, PDP activators, PGC1α polypeptides or variants thereof, and PGC1α agonists, for at least 15, 6, or 7 days, at least 2 weeks, at least 1, 2, 3, 4, 5, or 6 months or more with 1, 2, 3, 4, 5, 6, 7, 8, 9, or 10 or more rounds of expansion.

a. PDHK1 Inhibitors

As used herein, the term “PDHK1 inhibitor” refers to a nucleic acid, peptide, compound, antibody, antibody fragment, aptamer, or small organic molecule that inhibits at least one activity of PDHK1. In some examples, such an inhibitor specifically binds to PDHK1. In other examples, the inhibition of PDHK1 is indirect. Thus, PDHK1 inhibitors encompasses inhibitors that prevent PDHK1 from engaging with its downstream signaling pathway(s). Inhibition of PDHK1 affects a range of activities and cellular pathways. For example, inhibition of PDHK1 and/or its downstream signaling pathways may reduce or inhibit PDHK1-mediated mitochondrial PDH phosphorylation, increase oxidation of pyruvate in mitochondria, increase glucose in the mitochondria, and/or reduce or inhibit the conversion of pyruvate to lactate in the cytosol.

A PDHK1 inhibitor that only targets PDHK1 can be referred to as a selective PDHK1 inhibitor. In one embodiment, a selective PDHK1 inhibitor refers to an agent that exhibits a 50% inhibitory concentration with respect to PDHK1 that is at least 10-fold, at least 20-fold, at least 30-fold, at least 50-fold, at least 100-fold, at least 1000-fold, or more, lower than the inhibitor's IC50 with respect to PDH and/or other proteins in the PDHK1 pathway.

In a particular embodiment, exemplary PDHK1 inhibitors inhibit PDHK1 with an IC50 (concentration that inhibits 50% of the activity) of about 200 nM or less, such as about 100 nm or less, such as about 60 nM or less, about 25 nM, about 10 nM, about 5 nM, about 1 nM, 100 μM, 50 μM, 25 μM, 10 M, 1 M, or less. In one embodiment, a PDHK1 inhibitor inhibits PDHK1 with an IC50 from about 2 nM to about 100 nm, such as from about 2 nM to about 50 nM, such as from about 2 nM to about 15 nM.

Illustrative examples of PDHK1 inhibitors suitable for use in culturing T cells or other immune effector cells that have been activated (and in some examples stimulated), and transduced with an engineered antigen receptor such as TCR, DARIC, or CAR, using the manufacturing methods contemplated herein include, but are not limited to, a peptide, a PDHK1 antibody or antibody fragment, a small molecule, inhibitory RNA (RNAi) molecule, or aptamer. In some examples, the PDHK1 inhibitors comprises a PDHK1 inhibitory RNA (RNAi) molecule, a PDHK1 aptamer, or combinations thereof.

Non-limiting examples of useful PDHK1 inhibitors include, e.g., interfering RNA (e.g., siRNA), dsRNA, RNA polymerase III transcribed DNAs, ribozymes, and anti-sense nucleic acids. In one example, a PDHK1 inhibitor is an inhibitory RNA (RNAi), such as a PDHK1 antisense molecule, a PDHK1 siRNA molecule, a PDHK1 microRNA molecule, or combinations thereof.

PDHK1 antisense oligonucleotides, including antisense DNA, RNA, and DNA/RNA molecules, act to directly block the translation of PDHK1 mRNA by binding to PDHK1 mRNA and reducing or preventing PDHK1 protein translation. For example, PDHK1 antisense oligonucleotides of at least about 15 bases and complementary to unique regions of the PDHK1 DNA sequence (e.g., SEQ ID NO: 1) can be synthesized, e.g., by phosphodiester techniques (Dallas et al, (2006) Med Sci. Monit. 12(4):RA67-74; Kalota et al., (2006) Handb. Exp. Pharmacol. 173:173-96; Lutzelburger et al., (2006) Handb. Exp. Pharmacol. 173:243-59). In one example, an antisense oligonucleotide of at least 20 contiguous nucleotides of a PDHK1 DNA sequence (e.g., SEQ ID NO: I) and limits tissue expression of PDHK1. In one example, an antisense oligonucleotide may include a chimeric phosphorothioate antisense oligonucleotide. Exemplary phosphorothioate antisense oligonucleotides that can be used include: 1) 5′-AGCTGGCTGGTGGTCCTGGC-3′ (SEQ ID NO: 20) and, and 2) 5′-TGAATGATGCCCTTGCCGTG-3′ (SEQ ID NO: 21).

siRNA comprises a double stranded structure typically containing 15 to 50 base pairs, such as 21 to 27 or 21 to 25 base pairs and having a nucleotide sequence identical or nearly identical to a PDHK1 gene or RNA. Antisense polynucleotides include, but are not limited to: morpholinos, 2-O-methyl polynucleotides, DNA, RNA and the like.

In one example, a human PDK1 RNAi sequence comprises or consists of the sequence (1) GCTCTGTCAACAGACTCAATA (SEQ ID NO: 22), (2) CATCCGTTCAATTGGTACAAA (SEQ ID NO: 23), or (3) CGTGAATATGTTGAAGTAGAA (SEQ ID NO: 24).

RNA polymerase III transcribed DNAs contain promoters, such as the U6 promoter. These DNAs can be transcribed to produce small hairpin RNAs in the cell that can function as siRNA or linear RNAs that can function as antisense RNA. The inhibitor may be polymerized in vitro, recombinant RNA, contain chimeric sequences, or derivatives of these groups. The inhibitor may contain ribonucleotides, deoxyribonucleotides, synthetic nucleotides, or any suitable combination such that the target RNA and/or gene is inhibited. In addition, these forms of nucleic acid may be single, double, triple, or quadruple stranded. (see for example Bass (2001) Nature, 411, 428 429: Elbashir et al., (2001) Nature, 411, 494 498; and PCT Publication Nos. WO 00/44895, WO 01/36646, WO 99/32619, WO 00/01846, WO 01/29058, WO 99/07409, WO 00/44914).

Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hammerhead motif ribozyme molecules that specifically and efficiently catalyze endonucleolytic cleavage of mRNA sequences are also within the scope of the present invention. Scanning a PDHK1 nucleic acid molecule (e.g., SEQ ID NO: 1 or nt 44-1354 of SEQ ID NO: 1) for ribozyme cleavage sites that include the following sequences, GUA, GUU, and GUC initially identifies specific ribozyme cleavage sites within a PDHK1 RNA-target. Once identified, short RNA sequences of between about 15 and 20 ribonucleotides corresponding to the region of the PDHK1 gene containing the cleavage site can be evaluated for structural features such as secondary structure that may render the oligonucleotide sequence unsuitable. The suitability of candidate targets can also be evaluated by testing their accessibility to hybridization with complementary oligonucleotides using, e.g., ribonuclease protection assays.

PDHK1 expression inhibitors can be prepared by several methods. These include techniques for chemical synthesis such as, e.g., by solid phase phosphoamite chemical synthesis. Alternatively, antisense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. See, e.g., Weintraub, H. et al, Antisense RNA as a molecular tool for genetic analysis, Reviews—Trends in Genetics, Vol. 1 (1) 1986.

Various modifications to the PDHK1 inhibitory oligonucleotides can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Aptamer nucleic acid sequences can be made that inhibit PDHK1 activity. The aptamer nucleic acid sequences can be comprised entirely of RNA or partially of RNA, or entirely or partially of DNA and/or other nucleotide analogs. Aptamers are typically developed to bind particular ligands by employing known in vivo or in vitro (most typically, in vitro) selection techniques known as SELEX (Systematic Evolution of Ligands by Exponential Enrichment). Methods of making aptamers are described in, for example, Ellington and Szostak (1990) Nature 346:818, Tuerk and Gold (1990) Science 249:505, U.S. Pat. No. 5,582,981; PCT Publication No. WO 00/20040; U.S. Pat. No. 5,270,163; Lorsch and Szostak (1994) Biochem. 33:973; Mannironi et al., (1997) Biochem. 36:9726; Blind (1999) Proc. Nat'l. Acad. Sci. USA 96:3606-3610, Huizenga and Szostak (1995) Biochem. 34:656-665, PCT Publication Nos. WO 99/54506, WO 99/27133, and WO 97/42317; and U.S. Pat. No. 5,756,291.

In one example, a PDHK1 inhibitor is a PDHK1 small molecule, such as dichloroacetic acid (DCA), AZD7545, BX-912, BX-320, BX-795, or a combination thereof.

In one example, a PDHK1 inhibitor is a PDHK1 antibody or antibody fragment, such as one that specifically binds to an epitope of SEQ ID NO: 2 (or aa 29-436 of SEQ ID NO: 2). Exemplary PDHK1 commercially available antibodies include, but are not limited to sc-28783, HPA059083, and 1HPA027376.
b. PDP and PGC1α Activators

Activators of PDP include a PDP peptide, an enzyme, a PDP antibody, a PDP antibody fragment, a small molecule, or a combination of two or more thereof. Activators of PGC1α include a PGC1α peptide, an enzyme, a PGC1α antibody, a PGC1α antibody fragment, a small molecule, or a combination of two or more thereof.

In one example, the PDP activator is a peptide, such as insulin.

In one example, the PDP activator is a small molecule, such as PEP, AMP, or a combination thereof. In one example, PDP is activated via modulation of downstream signaling pathway(s).

In one example, the PGC1α activator is a small molecule, such as ZLN005 or mogroside VI B.

In one example, the PDP activator is a PDP protein, or nucleic acid encoding such. Thus, in some examples, an activated and transduced T cell or other immune effector cell is incubated with a PDP protein, such as one having at least at least 80%, at least 85%, at least 90%, at least 92%, at least 95%, at least 98%, at least 99%, or 100% sequence identity to SEQ ID NO: 4 or any PDP GenBank® Accession No. provided herein (or the immune effector cell is transformed with a PDP protein coding sequence).

Nucleic acid molecules encoding a native or variant PDP protein can be used as PDP activators, such as a nucleic acid molecule having at least 80%, at least 85%, at least 90%, at least 92%, at least 95%, at least 98%, or at least 99% sequence identity to SEQ ID NO: 3 or nt 202-1815 of SEQ ID NO: 3 or any PDP GenBank® Accession No. provided herein. In addition, one of skill can readily construct a variety of clones containing functionally equivalent nucleic acids, such as nucleic acids which differ in sequence but which encode the same protein sequence.

In one example, the PGC1α activator is a PGC1α protein, or nucleic acid encoding such. Thus, in some examples, an activated and transduced T cell or other immune effector cell is incubated with a PGC1α protein, such as one having at least at least 80%, at least 85%, at least 90%, at least 92%, at least 95%, at least 98%, at least 99%, or 100% sequence identity to SEQ ID NO: 18 or 19 or any PGC1α GenBank® Accession No. provided herein (or a nucleic acid encoding such a PGC1α protein). In one example, the PGC1α activator is a PGC1α polypeptide fragment (or nucleic acid encoding such), such as amino acids 1-270 of PGC1α (e.g., aa 1-270 of SEQ ID NO: 18 or 19), amino acids 1-290 of PGC1α (e.g., aa 1-290 of SEQ ID NO: 18 or 19), or no more than about the first (N-terminal) 270 or 290 amino acids of PGC1α (e.g., no more than about the first (N-terminal) 270 or 290 aa of SEQ ID NO: 18 or 19).

Nucleic acid molecules include DNA, cDNA and RNA sequences which encode a PDP or PGC1α peptide. Silent mutations in a PDP or PGC1α coding sequence result from the degeneracy (i.e., redundancy) of the genetic code, whereby more than one codon can encode the same amino acid residue. Thus, for example, leucine can be encoded by CTT, CTC, CTA, CTG, TTA, or TTG; serine can be encoded by TCT, TCC, TCA, TCG, AGT, or AGC; asparagine can be encoded by AAT or AAC; aspartic acid can be encoded by GAT or GAC; cysteine can be encoded by TGT or TGC; alanine can be encoded by GCT, GCC, GCA, or GCG; glutamine can be encoded by CAA or CAG; tyrosine can be encoded by TAT or TAC; and isoleucine can be encoded by ATT, ATC, or ATA.

Codon preferences and codon usage tables for a particular species can be used to engineer isolated nucleic acid molecules encoding a PDP or PGC1α protein that take advantage of the codon usage preferences of that particular species. For example, the PDP or PGC1α protein expressed from the vector(s) can be designed to have codons that are preferentially used by a particular organism of interest (e.g., in one whom the T cells are introduced).

A nucleic acid encoding a PDP or PGC1α protein can be cloned or amplified by in vitro methods, such as polymerase chain reaction (PCR). In addition, nucleic acids encoding sequences encoding a PDP or PGC1α protein can be prepared by cloning techniques. Examples of appropriate cloning and sequencing techniques, and instructions are found in Sambrook et al. (ed.), Molecular Cloning: A Laboratory Manual 2nd ed., vol. 1-3, Cold Spring Harbor Laboratory Press, Cold Spring, Harbor, N.Y., 1989, and Ausubel et al., (1987) in “Current Protocols in Molecular Biology,” John Wiley and Sons, New York, N.Y.

Nucleic acid sequences encoding a PDP or PGC1α protein can be prepared by any suitable method including, for example, cloning of appropriate sequences or by direct chemical synthesis by methods such as the phosphotriester method of Narang et al., Meth. Enzymol. 68:90-99, 1979; the phosphodiester method of Brown et al., Meth. Enzymol. 68:109-151, 1979; the diethylphosphoramidite method of Beaucage et al., Tetra. Lett. 22:1859-1862, 1981; the solid phase phosphoramidite triester method described by Beaucage & Caruthers, Tetra. Letts. 22(20):1859-1862, 1981, for example, using an automated synthesizer as described in, for example, Needham-VanDevanter et al., Nucl. Acids Res. 12:6159-6168, 1984; and, the solid support method of U.S. Pat. No. 4,458,066. Chemical synthesis produces a single stranded oligonucleotide. This can be converted into double stranded DNA by hybridization with a complementary sequence, or by polymerization with a DNA polymerase using the single strand as a template. While chemical synthesis of DNA is generally limited to sequences of about 100 bases, longer sequences may be obtained by the ligation of shorter sequences.

In some examples, such a sequence is optimized for expression in a host immune effector cell, such as a mammalian or human immune effector cell. Such a PDP or PGC1α coding sequence can be incorporated into a vector to allow for expression of PDP and/or PGC1α in an immune effector cell. The insertion can be made so that the PDP or PGC1α protein is read in frame so that the PDP or PGC1α protein is produced. Examples of vectors that can be used include plasmids, viral vectors, such as a lentiviral vector or retrovirus. The expression vector can contain additional elements necessary for the transfer and subsequent replication of the expression vector containing the PDP or PGC1α protein coding sequence in the cell. Examples of such elements include, but are not limited to, origins of replication and selectable markers, such as a thymidine kinase gene or an antibiotic resistance marker. In another example, a naked nucleic acid molecule encoding a PDP or PGC1α protein is used.

Nucleic acid sequences encoding a PDP or PGC1α protein can be operatively linked to expression control sequences, such as a native or no-native promoter. An expression control sequence operatively linked to PDP or PGC1α protein coding sequence is ligated such that expression of the PDP or PGC1α protein coding sequence is achieved under conditions compatible with the expression control sequences. Exemplary expression control sequences include, but are not limited to appropriate promoters, enhancers, transcription terminators, a start codon (i.e., ATG) in front of PDP or PGC1α protein, splicing signal for introns, maintenance of the correct reading frame of that gene to permit proper translation of mRNA, and stop codons. Examples of expression control elements that can be used include, but are not limited to, lac system, operator and promoter regions of phage lambda, and promoters derived from polyoma, adenovirus, retrovirus or SV40. Additional operational elements include, but are not limited to, leader sequence, termination codons, polyadenylation signals and any other sequences necessary for the appropriate transcription and subsequent translation of the nucleic acid sequence encoding the PDP or PGC1α protein in the T cell. In one example, the promoter is a 7.5 promoter. In one example, an IRES is used to drive expression. In some examples, two promoters are used.

Viral vectors can be prepared that encode a PDP or PGC1α protein (or both). Exemplary viral vectors that can be used include, but are not limited to, polyoma, SV40, adenovirus, vaccinia virus, adeno-associated virus, herpes viruses including HSV and EBV, Sindbis viruses, alphaviruses and retroviruses of avian, murine, and human origin. Baculovirus (Autographa californica multinuclear polyhedrosis virus; AcMNPV) vectors can also be used. Other suitable vectors include orthopox vectors, avipox vectors, fowlpox vectors, capripox vectors, suipox vectors, lentiviral vectors, alpha virus vectors, and poliovirus vectors. Specific exemplary vectors are poxvirus vectors such as vaccinia virus, fowlpox virus and a highly attenuated vaccinia virus (MVA), adenovirus, baculovirus and the like. Pox viruses of use include orthopox, suipox, avipox, and capripox virus. Orthopox include vaccinia, ectromelia, and raccoon pox. One example of an orthopox of use is vaccinia. Avipox includes fowlpox, canary pox and pigeon pox. Capripox include goatpox and sheeppox. In one example, the suipox is swinepox. Other viral vectors that can be used include other DNA viruses such as herpes virus and adenoviruses, and RNA viruses such as retroviruses and polio.

In another example, the PDP activator is a PDP antibody or antibody fragment, such as one that specifically binds to an epitope of SEQ ID NO: 3, 4, or nt 202-1815 of SEQ ID NO: 3. Commercially available PDP antibodies include 21176-1-AP, PA5-54338, HPA021152 (polyclonal), orb30461 (polyclonal), 21176-1-AP, PA5-54338, PA5-50477, ABIN954075, ABIN954075, NBP1-82432, NBP1-87308, NBP1-87308, HPA021152, HPA018483, LS-C405254, LS-C163497, 114139, 07-1223, and other commercially available antibodies. In one example, the PDP activator is an agonist PDP aptamer.

In another example, the PGC1α activator is a PGC1α antibody or antibody fragment, such as one that specifically binds to an epitope of SEQ ID NO:18 or 19. In one example, the PGC1α activator is an agonist PGC1α aptamer.

### IV. Engineered Antigen Receptors

The immune effector cell manufacturing methods contemplated are useful for expanding immune effector cells modified to express one or more engineered antigen receptors, such as high affinity T cell receptors (engineered TCRs), dimerizing agent regulated immunoreceptor complexes (DARIC), or chimeric antigen receptors (CARs) without a concomitant increase in the differentiation of these modified immune effector cells. In one embodiment, the immune effector cell is genetically modified to express one or more engineered antigen receptors, such as TCRs, DARICs, or CARs. As used herein, immune effector cells modified to express an engineered antigen receptor, such as TCR, DARIC, or CAR contemplated herein may be referred to as, “antigen-specific redirected immune effector cells.”

### A. Engineered TCRs

Naturally occurring T cell receptors comprise two subunits, an α-subunit and a β-subunit, each of which is a unique protein produced by recombination event in each T cell's genome. Libraries of TCRs may be screened for their selectivity to particular target antigens. In this manner, natural TCRs, which have a high-avidity and reactivity toward target antigens may be selected, cloned, and subsequently introduced into a population of T cells used for adoptive immunotherapy.

In one embodiment, immune effector cells (e.g., T cells) are modified by introducing a polynucleotide encoding a subunit of a TCR that has the ability to form TCRs that confer specificity to T cells for tumor cells expressing a target antigen. In particular embodiments, the subunits have one or more amino acid substitutions, deletions, insertions, or modifications compared to the naturally occurring subunit, so long as the subunits retain the ability to form TCRs conferring upon transfected immune effector cells (e.g., T cells) the ability to home to target cells, and participate in immunologically-relevant cytokine signaling. The engineered TCRs in some examples also bind target cells displaying the relevant tumor-associated peptide with high avidity, and optionally mediate efficient killing of target cells presenting the relevant peptide in vivo.

The nucleic acids encoding engineered TCRs can be isolated from their natural context in a (naturally-occurring) chromosome of a T cell, and can be incorporated into suitable vectors as described elsewhere herein. Both the nucleic acids and the vectors comprising them usefully can be transferred into an immune effector cell, such as a T cell. The modified immune effector cells (e.g., T cells) are then able to express one or more chains of a TCR (such as two chains) encoded by the transduced nucleic acid or nucleic acids. In some embodiments, the engineered TCR is an exogenous TCR because it is introduced into T cells that do not normally express the particular TCR. One aspect of the engineered TCRs is that it has high avidity for a tumor antigen presented by a major histocompatibility complex (MHC) or similar immunological component. In contrast to engineered TCRs, CARs are engineered to bind target antigens in an MHC independent manner.

The protein encoded by the nucleic acids can be expressed with additional polypeptides attached to the amino-terminal or carboxyl-terminal portion of the α-chain or β-chain of a TCR so long as the attached additional polypeptide does not interfere with the ability of the α-chain or β-chain to form a functional T cell receptor and the MHC dependent antigen recognition.

Antigens that are recognized by the engineered TCRs contemplated herein include, but are not limited to cancer antigens, including antigens on both hematological cancers and solid tumors. Illustrative antigens include, but are not limited to α-fetoprotein (AFP), B Melanoma Antigen (BAGE) family members, Brother of the regulator of imprinted sites (BORIS), Cancer-testis antigens, Cancer-testis antigen 83 (CT-83), Carbonic anhydrase IX (CAIX), Carcinoembryonic antigen (CEA), Cytomegalovirus (CMV) antigens, Cytotoxic T cell (CTL)-recognized antigen on melanoma (CAMEL), Epstein-Barr virus (EBV) antigens, G antigen 1 (GAGE-1), GAGE-2, GAGE-3, GAGE-4, GAGE-5, GAGE-6, GAGE-7B, GAGE-8, Glycoprotein 100 (GP100), Hepatitis B virus (HBV) antigens, Hepatitis C virus (HCV) non-structure protein 3 (NS3), Human Epidermal Growth Factor Receptor 2 (HER-2), Human papillomavirus (HPV)-E6, HPV-E7, Human telomerase reverse transcriptase (hTERT), Latent membrane protein 2 (LMP2), Melanoma antigen family A, 1 (MAGE-A1), MAGE-A2, MAGE-A3, MAGE-A4, MAGE-A6, MAGE-A10, MAGE-A12, Melanoma antigen recognized by T cells (MART-1), Mesothelin (MSLN), Mucin 1 (MUC1), Mucin 16 (MUC16), New York esophageal squamous cell carcinoma-1 (NYESO-1), P53,P antigen (PAGE) family members, Placenta-specific 1 (PLAC1), Preferentially expressed antigen in melanoma (PRAME), Survivin, Synovial sarcoma X 1 (SSX1), Synovial sarcoma X 2 (SSX2), Synovial sarcoma X 3 (SSX3), Synovial sarcoma X 4 (SSX4), Synovial sarcoma X 5 (SSX5), Synovial sarcoma X 8 (SSX8), Thyroglobulin, Tyrosinase, Tyrosinase related protein (TRP)1, TRP2, Wilms tumor protein (WT-1), X Antigen Family Member 1 (XAGE1), and X Antigen Family Member 2 (XAGE2).

### B. Chimeric Antigen Receptors (CARs)

The immune effector cell manufacturing methods contemplated herein include modifying T cells or other immune effector cells to express one or more CARs as contemplated herein. In various embodiments, the present disclosure provides immune effector cells (e.g., T cells) genetically engineered with vectors designed to express CARs that redirect cytotoxicity toward tumor cells. CARs are molecules that combine antibody-based specificity for a target antigen (e.g., tumor antigen) with a T cell receptor-activating intracellular domain to generate a chimeric protein that exhibits a specific anti-tumor cellular immune activity. As used herein, the term, “chimeric,” describes being composed of parts of different proteins or DNAs from different origins.

The CARs contemplated herein comprise an extracellular domain that binds to a specific target antigen (also referred to as a binding domain or antigen-specific binding domain), a transmembrane domain and an intracellular signaling domain. The main characteristic of CARs is their ability to redirect immune effector cell specificity, thereby triggering proliferation, cytokine production, phagocytosis or production of molecules that can mediate cell death of the target antigen expressing cell in a major histocompatibility (MHC) independent manner, exploiting the cell specific targeting abilities of monoclonal antibodies, soluble ligands or cell specific coreceptors.

In particular embodiments, a CAR includes an extracellular binding domain including but not limited to an antibody or antigen binding fragment thereof, a tethered ligand, or the extracellular domain of a coreceptor, that specifically binds a target antigen selected from the group consisting of alpha folate receptor (FRα), αᵥβ₆ integrin, B cell maturation antigen (BCMA), B7-H3 (CD276), B7-H6, carbonic anhydrase IX (CAIX), CD16, CD19, CD20, CD22, CD30, CD33, CD37, CD38, CD44, CD44v6, CD44v7/8, CD70, CD79a, CD79b, CD123, CD133, CD138, CD171, carcinoembryonic antigen (CEA), claudin 6, (CLDN6), claudin 1_8 isoform 2 (CLDN18.2), C-type lectin-like molecule-1 (CLL-1), CD2 subset 1 (CS-1), chondroitin sulfate proteoglycan 4 (CSPG4), cutaneous T cell lymphoma-associated antigen 1 (CTAGE1), delta like canonical Notch ligand 3 (DLL3), epidermal growth factor receptor (EGFR), epidermal growth factor receptor variant III (EGFRvIII), epithelial glycoprotein 2 (EGP2), epithelial glycoprotein 40 (EGP40), epithelial cell adhesion molecule (EPCAM), ephrin type-A receptor 2 (EPLA2), erb-b2 receptor tyrosine kinase 4 (ERBB4), fibroblast activation protein (FAP), Fc Receptor Like 5 (FCRL5), fetal acetylcholinesterase receptor (AchR), ganglioside G2 (GD2), ganglioside G3 (GD3), Glypican-3 (GPC3), EGFR family including ErbB2 (HER2), HER2 p95, IL-10Rα, IL-13Rα2, Kappa, cancer/testis antigen 2 (LAGE-1A), Lambda, Lewis-Y (LeY), L1 cell adhesion molecule (L1-CAM), melanoma antigen gene (MAGE)-A1, MAGE-A3, MAGE-A4, MAGE-A6, MAGEA10, melanoma antigen recognized by T cells 1 (MelanA or MART1), Mesothelin (MSLN), MUC1, MUC16, MHC class I chain related proteins A (MICA), MHC class I chain related proteins B (MICB), neural cell adhesion molecule (NCAM), cancer/testis antigen 1 (NY-ESO-1), polysialic acid; placenta-specific 1 (PLAC1), preferentially expressed antigen in melanoma (PRAME), prostate stem cell antigen (PSCA), prostate-specific membrane antigen (PSMA), receptor tyrosine kinase-like orphan receptor 1 (ROR1), synovial sarcoma, X breakpoint 2 (SSX2), Survivin, tumor associated glycoprotein 72 (TAG72), tumor endothelial marker 1 (TEM1/CD248), tumor endothelial marker 7-related (TEM7R), trophoblast glycoprotein (TPBG), UL16-binding protein (ULBP) 1, ULBP2, ULBP3, ULBP4, ULBP5, ULBP6, vascular endothelial growth factor receptor 2 (VEGFR2), and Wilms tumor 1 (WT-1); a transmembrane domain including, but not limited to, transmembrane domains from CD8α, CD28, CD4, CD45, PD-1, and CD152; one or more intracellular costimulatory signaling domains including but not limited to intracellular costimulatory signaling domains from CD28, CD54 (ICAM), CD134 (OX40), CD137 (41BB), CD152 (CTLA4), CD273 (PD-L2), CD274 (PD-L1), and CD278 (ICOS); and a primary signaling domain from CD3ζ or FcRγ. In some examples, the extracellular binding domain binds BCMA.

In some examples, a CAR further comprises a signal peptide, such as an IgG1 heavy chain signal polypeptide or a CD8α signal polypeptide.

### 1. Binding Domain

In particular embodiments, CARs contemplated herein include an extracellular binding domain that specifically binds to a target polypeptide, e.g., target antigen, expressed on tumor cell. As used herein, the terms, “binding domain,” “extracellular domain,” “extracellular binding domain,” “antigen-specific binding domain,” and “extracellular antigen specific binding domain,” are used interchangeably and provide a CAR with the ability to specifically bind to the target antigen of interest. A binding domain may include any protein, polypeptide, oligopeptide, or peptide that possesses the ability to specifically recognize and bind to a biological molecule (e.g., a cell surface receptor or tumor protein, lipid, polysaccharide, or other cell surface target molecule, or component thereof). A binding domain includes any naturally occurring, synthetic, semi-synthetic, or recombinantly produced binding partner for a biological molecule of interest.

In particular embodiments, the extracellular binding domain of a CAR includes an antibody or antigen binding fragment thereof. An “antibody” refers to a binding agent that is a polypeptide comprising at least a light chain or heavy chain immunoglobulin variable region which specifically recognizes and binds an epitope of a target antigen, such as a peptide, lipid, polysaccharide, or nucleic acid containing an antigenic determinant, such as those recognized by an immune cell. Antibodies include antigen binding fragments thereof. The term also includes genetically engineered forms such as chimeric antibodies (for example, humanized murine antibodies), heteroconjugate antibodies (such as, bispecific antibodies) and antigen binding fragments thereof. See also, Pierce Catalog and Handbook, 1994-1995 (Pierce Chemical Co., Rockford, IL); Kuby, J., Immunology, 3rd Ed., W. H. Freeman & Co., New York, 1997.

In particular embodiments, the target antigen is an epitope of an alpha folate receptor (FRα), αᵥβ₆ integrin, B cell maturation antigen (BCMA), B7-H3 (CD276), B7-16, carbonic anhydrase IX (CAIX), CD16, CD19, CD20, CD22, CD30, CD33, CD37, CD38, CD44, CD44v6, CD44v7/8, CD70, CD79a, CD79b, CD123, CD133, CD138, CD171, carcinoembryonic antigen (CEA), claudin 6, (CLDN6), claudin 18 isoform 2 (CLDN18.2), C-type lectin-like molecule-1 (CLL-1), CD2 subset 1 (CS-1), chondroitin sulfate proteoglycan 4 (CSPG4), cutaneous T cell lymphoma-associated antigen 1 (CTAGE1), delta like canonical Notch ligand 3 (DLL3), epidermal growth factor receptor (EGFR), epidermal growth factor receptor variant III (EGFRvIII), epithelial glycoprotein 2 (EGP2), epithelial glycoprotein 40 (EGP40), epithelial cell adhesion molecule (EPCAM), ephrin type-A receptor 2 (EPHA2), erb-b2 receptor tyrosine kinase 4 (ERBB4), fibroblast activation protein (FAP), Fc Receptor Like 5 (FCRL5), fetal acetylcholinesterase receptor (AchR), ganglioside G2 (GD2), ganglioside GT3 (GD3), Glypican-3 (GPC3), EGFR family including ErbB2 (HER2), HER2 p95, IL-10Rα, IL-13Rα2, Kappa, cancer/testis antigen 2 (LAGE-1A), Lambda, Lewis-Y (LeY), L1 cell adhesion molecule (L1-CAM), melanoma antigen gene (MAGE)-A1, MAGE-A3, MAGE-A4, MAGE-A6, MAGEA10, melanoma antigen recognized by T cells 1 (MelanA or MART1), Mesothelin (MSLN), MUC1, MUC16, MHC class I chain related proteins A (MICA), MHC class I chain related proteins B (MICB), neural cell adhesion molecule (NCAM), cancer/testis antigen 1 (NY-ESO-1), polysialic acid; placenta-specific 1 (PLAC1), preferentially expressed antigen in melanoma (PRAME), prostate stem cell antigen (PSCA), prostate-specific membrane antigen (PSMA), receptor tyrosine kinase-like orphan receptor 1 (ROR1), synovial sarcoma, X breakpoint 2 (SSX2), Survivin, tumor associated glycoprotein 72 (TAG72), tumor endothelial marker 1 (TEM1/CD248), tumor endothelial marker 7-related (TEM7R), trophoblast glycoprotein (TPBG), UL16-binding protein (ULBP) 1, ULBP2, ULBP3, ULBP4, ULBP5, ULBP6, vascular endothelial growth factor receptor 2 (VEGFR2), or Wilms tumor 1 (WT-1).

Light and heavy chain variable regions contain a “framework” region interrupted by three hypervariable regions, also called “complementarity-determining regions” or “CDRs.” The CDRs can be defined or identified by conventional methods, such as by sequence according to Kabat et al (Wu, TT and Kabat, E. A., J Exp Med. 132(2):211-50, (1970); Borden, P. and Kabat E. A., PNAS, 84: 2440-2443 (1987); (see, Kabat et al., Sequences of Proteins of Immunological Interest, U.S. Department of Health and Human Services, 1991, which is hereby incorporated by reference), or by structure according to Chothia et al (Choithia, C. and Lesk, A. M., J Mol. Biol., 196(4): 901-917 (1987), Choithia, C. et al, Nature, 342: 877-883 (1989)).

The sequences of the framework regions of different light or heavy chains are relatively conserved within a species, such as humans. The framework region of an antibody, that is the combined framework regions of the constituent light and heavy chains, serves to position and align the CDRs in three-dimensional space. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, the CDRs located in the variable domain of the heavy chain of the antibody are referred to as CDRH1, CDRH2, and CDRH3, whereas the CDRs located in the variable domain of the light chain of the antibody are referred to as CDRL1, CDRL2, and CDRL3. Antibodies with different specificities (i.e., different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

References to “VH” or “VH” refer to the variable region of an immunoglobulin heavy chain, including that of an antibody, Fv, scFv, dsFv, Fab, or other antibody fragment as disclosed herein. References to “VL” or “VL” refer to the variable region of an immunoglobulin light chain, including that of an antibody, Fv, scFv, dsFv, Fab, or other antibody fragment as disclosed herein.

A “monoclonal antibody” is an antibody produced by a single clone of B lymphocytes or by a cell into which the light and heavy chain genes of a single antibody have been transfected. Monoclonal antibodies can be produced by making hybrid antibody-forming cells from a fusion of myeloma cells with immune spleen cells. Monoclonal antibodies include humanized monoclonal antibodies.

A “chimeric antibody” has framework residues from one species, such as human, and CDRs (which generally confer antigen binding) from another species, such as a mouse. In particular embodiments, a CAR include an antigen-specific binding domain that is a chimeric antibody or antigen binding fragment thereof.

In certain embodiments, the antibody is a humanized antibody (such as a humanized monoclonal antibody) that specifically binds to a surface protein on a tumor cell. A “humanized” antibody is an immunoglobulin including a human framework region and one or more CDRs from a non-human (for example a mouse, rat, or synthetic) immunoglobulin. Humanized antibodies can be constructed by means of genetic engineering (see for example, U.S. Pat. No. 5,585,089).

In some examples, the extracellular domain of a CAR includes an antibody or antigen binding fragment that specifically binds to the antigen, such as a camel Ig, a llama Ig, shark Ig, an Alpaca Ig, Ig NAR, a Fab fragment, a F(ab′)2 fragment, a bispecific Fab dimer (Fab2), a trispecific Fab trimer (Fab3), an Fv, an single chain Fv protein (“scFv”), a bis-scFv, (scFv)2, a minibody, a diabody, a triabody, a tetrabody, a disulfide stabilized Fv protein (“dsFv”), or a single-domain antibody (sdAb, a camelid VHH, nanobody).

“Camel Ig” or “camelid VHH” as used herein refers to the smallest known antigen-binding unit of a heavy chain antibody (Koch-Nolte, et al, FASEB J., 21: 3490-3498 (2007)). A “heavy chain antibody” or a “camelid antibody” refers to an antibody that contains two VH domains and no light chains (Riechmann L. et al, J. Immunol. Methods 231:25-38 (1999); WO94/04678; WO94/25591; U.S. Pat. No. 6,005,079).

“IgNAR” of “immunoglobulin new antigen receptor” refers to class of antibodies from the shark immune repertoire that consist of homodimers of one variable new antigen receptor (VNAR) domain and five constant new antigen receptor (CNAR) domains.

Papain digestion of antibodies produces two identical antigen-binding fragments, called “Fab” fragments, each with a single antigen-binding site, and a residual “Fc” fragment, whose name reflects its ability to crystallize readily. The Fab fragment contains the heavy- and light-chain variable domains and also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

“Fv” is the minimum antibody fragment which contains a complete antigen-binding site. In a single-chain Fv (scFv) species, one heavy- and one light-chain variable domain can be covalently linked by a flexible peptide linker such that the light and heavy chains can associate in a “dimeric” structure analogous to that in a two-chain Fv species.

The term “diabodies” refers to antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies may be bivalent or bispecific. Diabodies are described more fully in, for example, EP 404,097; WO 1993/01161; Hudson et al., Nat. Med 9:129-134 (2003); and Hollinger et al., PNAS USA 90: 6444-6448 (1993). Triabodies and tetrabodies are also described in Hudson et al., Nat. Med. 9:129-134 (2003).

“Single domain antibody” or “sdAb” or “nanobody” refers to an antibody fragment that consists of the variable region of an antibody heavy chain (VH domain) or the variable region of an antibody light chain (VL domain) (Holt, L., et al, Trends in Biotechnology, 21(11): 484-490).

“Single-chain Fv” or “scFv” antibody fragments comprise the VH and VL domains of antibody, wherein these domains are present in a single polypeptide chain and in either orientation (e.g., VL-VH or VH-VL). Generally, the scFv polypeptide further comprises a polypeptide linker between the VH and VL domains which enables the scFv to form the desired structure for antigen binding. For a review of scFv, see, e.g., Pluckthün, in The Pharmacology of Monoclonal Antibodies, vol. 113, Rosenburg and Moore eds., (Springer-Verlag, New York, 1994), pp. 269-315.

In some embodiments, a CAR contemplated herein includes an antigen-specific binding domain that is an scFv (a murine, human or humanized scFv) that binds an antigen expressed on a cancer cell. In a certain embodiment, the scFv binds an antigen selected from the group consisting of alpha folate receptor (FRα), αᵥβ₆ integrin, B cell maturation antigen (BCMA), B7-H3 (CD276), B7-H6, carbonic anhydrase IX (CAIX), CD16, CD19, CD20, CD22, CD30, CD33, CD37, CD38, CD44, CD44v6, CD44v7/8, CD70, CD79a, CD79b, CD123, CD133, CD138, CD171, carcinoembryonic antigen (CEA), claudin 6, (CLDN6), claudin 18 isoform 2 (CLDN18.2), C-type lectin-like molecule-1 (CLL-1), CD2 subset 1 (CS-1), chondroitin sulfate proteoglycan 4 (CSPG4), cutaneous T cell lymphoma-associated antigen 1 (CTAGE1), delta like canonical Notch ligand 3 (DLL3), epidermal growth factor receptor (EGFR), epidermal growth factor receptor variant III (EGFRvIII), epithelial glycoprotein 2 (EGP2), epithelial glycoprotein 40 (EGP40), epithelial cell adhesion molecule (EPCAM), ephrin type-A receptor 2 (EPHA2), erb-b2 receptor tyrosine kinase 4 (ERBB4), fibroblast activation protein (FAP), Fc Receptor Like 5 (FCRL5), fetal acetylcholinesterase receptor (AchR), ganglioside G2 (GD2), ganglioside G3 (GD3), Glypican-3 (GPC3), EGFR family including ErbB2 (HER2), HER2 p95, IL-10Rα, IL-13Rα2, Kappa, cancer/testis antigen 2 (LAGE-1A), Lambda, Lewis-Y (LeY), L1 cell adhesion molecule (L1-CAM), melanoma antigen gene (MAGE)-A1, MAGE-A3, MAGE-A4, MAGE-A6, MAGEA10, melanoma antigen recognized by T cells 1 (MelanA or MART1), Mesothelin (MSLN), MUC1, MUC16, MHC class I chain related proteins A (MICA), MHC class I chain related proteins B (MICB), neural cell adhesion molecule (NCAM), cancer/testis antigen 1 (NY-ESO-1), polysialic acid; placenta-specific 1 (PLAC1), preferentially expressed antigen in melanoma (PRAME), prostate stein cell antigen (PSCA), prostate-specific membrane antigen (PSMA), receptor tyrosine kinase-like orphan receptor 1 (ROR1), synovial sarcoma, X breakpoint 2 (SSX2), Survivin, tumor associated glycoprotein 72 (TAG72), tumor endothelial marker 1 (TEM/CD248), tumor endothelial marker 7-related (TEM7R), trophoblast glycoprotein (TPBG), UL16-binding protein (ULBP) 1, ULBP2, ULBP3, ULBP4, UBPS, ULBP6, vascular endothelial growth factor receptor 2 (VEGFR2), and Wilms tumor 1 (WT-1).

In some examples, the CAR includes an extracellular binding domain that binds B-cell maturation antigen (BCMA, e.g., OMIM 109545). In some examples, the CAR includes one or more antibody fragments that bind BCMA. In some examples, the CAR includes one or more antibody fragments that bind BCMA, a CD8α hinge and a CD8α transmembrane domain, a 4-1BB costimulatory domain, and a CD3ζ primary signaling domain.

### 2. Linkers

In certain embodiments, the CARs contemplated herein may include linker residues between the various domains, e.g., between V_{H} and V_{L} domains, added for appropriate spacing and conformation of the molecule. CARs contemplated herein, may comprise one, two, three, four, or five or more linkers. In particular embodiments, the length of a linker is about 1 to about 25 amino acids, about 5 to about 20 amino acids, or about 10 to about 20 amino acids, or any intervening length of amino acids. In some embodiments, the linker is 1, 2, 3, 4, 5, 6, 7, 8, 9, 10, 11, 12, 13, 14, 15, 16, 17, 18, 19, 20, 21, 22, 23, 24, 25, or more amino acids long.

Illustrative examples of linkers include glycine polymers (G)ₙ; glycine-serine polymers (G₁₋₅S₁₋₅)ₙ, where n is an integer of at least one, two, three, four, or five; glycine-alanine polymers; alanine-serine polymers; and other flexible linkers known in the art. Glycine and glycine-serine polymers are relatively unstructured, and therefore may be able to serve as a neutral tether between domains of fusion proteins such as the CARs described herein. Glycine accesses significantly more phi-psi space than even alanine, and is much less restricted than residues with longer side chains (see Scheraga, Rev. Computational Chem. 11173-142 (1992)). The ordinarily skilled artisan will recognize that design of a CAR in particular embodiments can include linkers that are all or partially flexible, such that the linker can include a flexible linker as well as one or more portions that confer less flexible structure to provide for a desired CAR structure.

Other exemplary linkers include, but are not limited to the following amino acid sequences: GGG; DGGGS (SEQ ID NO: 8); TGEKP (SEQ ID NO: 9) (see, e.g., Liu et al., PNAS 5525-5530 (1997)); GGRR (SEQ ID NO: 10) (Pomerantz et al. 1995, supra); (GGGGS)ₙ wherein=1, 2, 3, 4 or 5 (SEQ ID NO: 11) (Kim et al., PNAS 93, 1156-1160 (1996.); EGKSSGSGSESKVD (SEQ ID NO: 12) (Chaudhary et al., 1990, Proc. Natl. Acad. Sci. U.S.A. 87:1066-1070); KESGSVSSEQLAQFRSLD (SEQ ID NO: 13) (Bird et al., 1988, Science 242:423-426), GGRRGGGS (SEQ ID NO: 14); LRQRDGERP (SEQ ID NO: 15); LRQKDGGGSERP (SEQ ID NO: 16); and LRQKD(GGGS)₂ ERP (SEQ ID NO: 17). Alternatively, flexible linkers can be rationally designed using a computer program capable of modeling both DNA-binding sites and the peptides themselves (Desjarlais & Berg, PNAS 90:2256-2260 (1993), PNAS 91:11099-11103 (1994) or by phage display methods.

In particular embodiments a CAR includes a scFV that further comprises a variable region linking sequence. A “variable region linking sequence,” is an amino acid sequence that connects a heavy chain variable region to a light chain variable region and provides a spacer function compatible with interaction of the two sub-binding domains so that the resulting polypeptide retains a specific binding affinity to the same target molecule as an antibody that comprises the same light and heavy chain variable regions. In one embodiment, the variable region linking sequence is 1, 2, 3, 4, 5, 6, 7, 8, 9, 10, 11, 12, 13, 14, 15, 16, 17, 18, 19, 20, 21, 22, 23, 24, 25, or more amino acids long. In a particular embodiment, the variable region linking sequence comprises a glycine-serine polymer (G₁₋₅S₁₋₅)ₙ, where n is an integer of at least 1, 2, 3, 4, or 5. In another embodiment, the variable region linking sequence comprises a (G₄S)₃ amino acid linker.

### 3. Spacer Domain

In particular embodiments, the binding domain of the CAR is followed by one or more “spacer domains,” which refers to the region that moves the antigen binding domain away from the effector cell surface to enable proper cell/cell contact, antigen binding and activation (Patel et al., Gene Therapy, 1999; 6: 412-419). The spacer domain may be derived either from a natural, synthetic, semi-synthetic, or recombinant source. In certain embodiments, a spacer domain is a portion of an immunoglobulin, including, but not limited to, one or more heavy chain constant regions, e.g., CH2 and CH3. The spacer domain can include the amino acid sequence of a naturally occurring immunoglobulin hinge region or an altered immunoglobulin hinge region.

In one embodiment, the spacer domain comprises the CH2 and CH3 of IgG1.

### 4. Hinge Domain

The binding domain of the CAR is generally followed by one or more “hinge domains,” which plays a role in positioning the antigen binding domain away from the effector cell surface to enable proper cell/cell contact, antigen binding and activation. A CAR generally comprises one or more hinge domains between the binding domain and the transmembrane domain (TM). The hinge domain may be derived either from a natural, synthetic, semi-synthetic, or recombinant source. The hinge domain can include the amino acid sequence of a naturally occurring immunoglobulin hinge region or an altered immunoglobulin hinge region.

Illustrative hinge domains suitable for use in the CARs described herein include the hinge region derived from the extracellular regions of type 1 membrane proteins such as CD8α, CD4, CD28 and CD7, which may be wild-type hinge regions from these molecules or may be altered. In another embodiment, the hinge domain comprises a CD8α hinge region.

In one example, the hinge region polypeptide comprises a hinge region or portion thereof of IgG2, IgG4, IgG1, CD28, CD8α, or a suitable combination thereof.

### 5. Transmembrane (TM) Domain

The “transmembrane domain” is the portion of the CAR that fuses the extracellular binding portion and intracellular signaling domain and anchors the CAR to the plasma membrane of the immune effector cell. The TM domain may be derived either from a natural, synthetic, semi-synthetic, or recombinant source.

Illustrative TM domains may be derived from (i.e., include at least the transmembrane region(s) of) the alpha, beta or zeta chain of the T-cell receptor, CD3 epsilon, CD3 zeta, CD4, CD5, CD8, CD9, CD16, CD22, CD27, CD28, CD33, CD37, CD45, CD64, CD80, CD86, CD134, CD137, CD152, and CD154. In some embodiments, the transmembrane domain is derived from a polypeptide selected from the group consisting of: CD8α, CD4, CD28, CD45, PD-1, and CD152. In some examples, the transmembrane domain is derived from CD8α or CD28.

In one embodiment, the CARs contemplated herein include a TM domain derived from CD8α. In another embodiment, a CAR contemplated herein include a TM domain derived from CD8α and a short oligo- or polypeptide linker, for example between 1, 2, 3, 4, 5, 6, 7, 8, 9, or 10 amino acids in length that links the TM domain and the intracellular signaling domain of the CAR. A glycine-serine linker provides a particularly suitable linker.

### 6. Intracellular Signaling Domain

In particular embodiments, CARs contemplated herein include an intracellular signaling domain. An “intracellular signaling domain,” refers to the part of a CAR that participates in transducing the message of effective CAR binding to a target antigen into the interior of the immune effector cell to elicit effector cell function, e.g., activation, cytokine production, proliferation and cytotoxic activity, including the release of cytotoxic factors to the CAR-bound target cell, or other cellular responses elicited with antigen binding to the extracellular CAR domain.

The term “effector function” refers to a specialized function of the cell. Effector function of the T cell, for example, may be cytolytic activity or help or activity including the secretion of a cytokine. Thus, the term “intracellular signaling domain” refers to the portion of a protein which transduces the effector function signal and that directs the cell to perform a specialized function. While usually the entire intracellular signaling domain can be employed, in many cases it is not necessary to use the entire domain. To the extent that a truncated portion of an intracellular signaling domain is used, such truncated portion may be used in place of the entire domain as long as it transduces the effector function signal. The term intracellular signaling domain is meant to include any truncated portion of the intracellular signaling domain sufficient to transducing effector function signal.

Signals generated through the TCR alone are insufficient for full activation of the T cell or other immune effector cell and that a secondary or costimulatory signal is also required. Thus, immune effector cell (e.g., T cell) activation can be said to be mediated by two distinct classes of intracellular signaling domains: primary signaling domains that initiate antigen-dependent primary activation through the TCR (e.g., a TCR/CD3 complex) and costimulatory signaling domains that act in an antigen-independent manner to provide a secondary or costimulatory signal. In some embodiments, a CAR contemplated herein comprises an intracellular signaling domain that comprises one or more “costimulatory signaling domain” and a “primary signaling domain.”

Primary signaling domains regulate primary activation of the TCR complex either in a stimulatory way, or in an inhibitory way. Primary signaling domains that act in a stimulatory manner may contain signaling motifs, known as immunoreceptor tyrosine-based activation motifs or ITAMs.

Illustrative examples of ITAM containing primary signaling domains include those derived from TCRζ, FcRγ, FcRβ, CD3γ, CD3δ, CD3ε, CD3ζ, CD22, CD79a, CD79b, and CD66d. In particular embodiments, a CAR includes a CD3ζ primary signaling domain and one or more costimulatory signaling domains. The intracellular primary signaling and costimulatory signaling domains may be linked in any order in tandem to the carboxyl terminus of the transmembrane domain.

CARs contemplated herein include one or more costimulatory signaling domains to enhance the efficacy and expansion of T cells or other immune effector cells expressing CAR receptors. As used herein, the term, “costimulatory signaling domain,” or “costimulatory domain”, refers to an intracellular signaling domain of a costimulatory molecule.

Illustrative examples of such costimulatory molecules include CD27, CD28, 4-1BB (CD137), OX40 (CD134), CD30, CD40, PD-1, ICOS (CD278), CTLA4, LFA-1, CD2, CD7, LIGHT, TRIM, LCK3, SLAM, DAP10, LAG3, HVEM, CD54 (ICAM), CD152 (CTLA4), CD273 (PD-L2), CD274 (PD-L1), NKD2C, and CD83. In some embodiments, the one or more intracellular costimulatory signaling domains are selected from the group consisting of CD54 (ICAM), CD134 (OX40), CD137 (41BB), CD152 (CTLA4), CD273 (PD-L2), CD274 (PD-L1), and CD278 (ICOS). In some examples, the group consists of CD28, CD134, and CD137. In one embodiment, a CAR includes one or more costimulatory signaling domains selected from the group consisting of CD28, CD137, CD134, and a CD3ζ primary signaling domain.

In one embodiment, a CAR includes an scFv that binds an alpha folate receptor (FRα), αᵥβ₆ integrin, B cell maturation antigen (BCMA), B7-13 (CD276), B7-H6, carbonic anhydrase IX (CAIX), CD16, CD19, CD20, CD22, CD30, CD33, CD37, CD38, CD44, CD44v6, CD44v7/8, CD70, CD79a, CD79b, CD123, CD133, CD138, CD171, carcinoembryonic antigen (CEA), claudin 6, (CLDN6), claudin 18 isoform 2 (CLDN18.2), C-type lectin-like molecule-1 (CLL-1), CD2 subset 1 (CS-1), chondroitin sulfate proteoglycan 4 (CSPG4), cutaneous T cell lymphoma-associated antigen 1 (CTAGE1), delta like canonical Notch ligand 3 (DLL3), epidermal growth factor receptor (EGFR), epidermal growth factor receptor variant III (EGFRvIII), epithelial glycoprotein 2 (EGP2), epithelial glycoprotein 40 (EGP40), epithelial cell adhesion molecule (EPCAM), ephrin type-A receptor 2 (EPHA2), erb-b2 receptor tyrosine kinase 4 (ERBB4), fibroblast activation protein (FAP), Fc Receptor Like 5 (FCRL5), fetal acetylcholinesterase receptor (AchR), ganglioside G2 (GD2), ganglioside G3 (GD3), Glypican-3 (GPC3), EGFR family including ErbB2 (HER2), HER2 p95, IL-10Rα, IL-13Rα2, Kappa, cancer/testis antigen 2 (LAGE-1A), Lambda, Lewis-Y (LeY), L1 cell adhesion molecule (L1-CAM), melanoma antigen gene (MAGE)-A1, MAGE-A3, MAGE-A4, MAGE-A6, MAGEA10, melanoma antigen recognized by T cells 1 (MelanA or MART1), Mesothelin (MSLN), MUC1, MUC16, MHC class I chain related proteins A (MICA), MHC class I chain related proteins B (MICB), neural cell adhesion molecule (NCAM), cancer/testis antigen 1 (NY-ESO-1), polysialic acid; placenta-specific 1 (PLAC1), preferentially expressed antigen in melanoma (PRAME), prostate stem cell antigen (PSCA), prostate-specific membrane antigen (PSMA), receptor tyrosine kinase-like orphan receptor 1 (ROR1), synovial sarcoma, X breakpoint 2 (SSX2), Survivin, tumor associated glycoprotein 72 (TAG72), tumor endothelial marker 1 (TEM1/CD248), tumor endothelial marker 7-related (TEM7R), trophoblast glycoprotein (TPBG), UL16-binding protein (ULBP) 1, ULBP2, ULBP3, ULBP4, ULBP5, ULBP6, vascular endothelial growth factor receptor 2 (VEGFR2), or Wilms tumor 1 (WT-1) polypeptide; a transmembrane domain derived from a polypeptide selected from the group consisting of: CD8α; CD4, CD45, PD-1, and CD152; and one or more intracellular costimulatory signaling domains selected from the group consisting of: CD28, CD54, CD134, CD137, CD152, CD273, CD274, and CD278; and a CD3ζ primary signaling domain.

In another embodiment, a CAR includes an scFv that binds an alpha folate receptor (FRα), αᵥβ₆ integrin, B cell maturation antigen (BCMA), B7-H3 (CD276), B7-H6, carbonic anhydrase IX (CAIX), CD16, CD19, CD20, CD22, CD30, CD33, CD37, CD38, CD44, CD44v6, CD44v7/8, CD70, CD79a, CD79b, CD123, CD133, CD138, CD171, carcinoembryonic antigen (CEA), claudin 6, (CLDN6), claudin 18 isoform 2 (CLDN18.2), C-type lectin-like molecule-1 (CLL-1), CD2 subset 1 (CS-1), chondroitin sulfate proteoglycan 4 (CSPG4), cutaneous T cell lymphoma-associated antigen 1 (CTAGE1), delta like canonical Notch ligand 3 (DLL3), epidermal growth factor receptor (EGFR), epidermal growth factor receptor variant III (EGFRvIII), epithelial glycoprotein 2 (EGP2), epithelial glycoprotein 40 (EGP40), epithelial cell adhesion molecule (EPCAM), ephrin type-A receptor 2 (EPHA2), erb-b2 receptor tyrosine kinase 4 (ERBB4), fibroblast activation protein (FAP), Fc Receptor Like 5 (FCRL5), fetal acetylcholinesterase receptor (AchR), ganglioside G2 (GD2), ganglioside G3 (GD3), Glypican-3 (GPC3), EGFR family including ErbB2 (HER2), HER2 p95, IL-10Rα, IL-13Rα2, Kappa, cancer/testis antigen 2 (LAGE-1A), Lambda, Lewis-Y (LeY), L1 cell adhesion molecule (L1-CAM), melanoma antigen gene (MAGE)-A1, MAGE-A3, MAGE-A4, MAGE-A6, MAGEA10, melanoma antigen recognized by T cells 1 (MelanA or MART1), Mesothelin (MSLN), MUC1, MUC16, MHC class I chain related proteins A (MICA), MHC class I chain related proteins B (MICB), neural cell adhesion molecule (NCAM), cancer/testis antigen 1 (NY-ESO-1), polysialic acid; placenta-specific 1 (PLAC1), preferentially expressed antigen in melanoma (PRAME), prostate stem cell antigen (PSCA), prostate-specific membrane antigen (PSMA), receptor tyrosine kinase-like orphan receptor 1 (ROR1), synovial sarcoma, X breakpoint 2 (SSX2), Survivin, tumor associated glycoprotein 72 (TAG72), tumor endothelial marker 1 (TEM1/CD248), tumor endothelial marker 7-related (TEM7R), trophoblast glycoprotein (TPBG), UL16-binding protein (ULBP) 1, ULBP2, ULBP3, ULBP4, ULBP5, ULBP6, vascular endothelial growth factor receptor 2 (VEGFR2), or Wilms tumor 1 (WT-1); a hinge domain selected from the group consisting of IgG1 hinge/CH2/CH3 and CD8α, and CD8α; a transmembrane domain derived from a polypeptide selected from the group consisting of CD8α; CD4, CD45, PD-1, and CD152; and one or more intracellular costimulatory signaling domains selected from the group consisting of CD28, CD134, and CD137; and a CD3ζ primary signaling domain.

In yet another embodiment, a CAR includes an scFv, further including a linker, that binds an alpha folate receptor (FRα), αᵥβ₆ integrin, B cell maturation antigen (BCMA), B7-H3 (CD276), B7-H6, carbonic anhydrase IX (CAIX), CD16, CD19, CD20, CD22, CD30, CD33, CD37, CD38, CD44, CD44v6, CD44v7/8, CD70, CD79a, CD79b, CD123, CD133, CD138, CD171, carcinoembryonic antigen (CEA), claudin 6, (CLDN6), claudin 18 isoform 2 (CLDN18.2), C-type lectin-like molecule-1 (CLL-1), CD2 subset 1 (CS-1), chondroitin sulfate proteoglycan 4 (CSPG4), cutaneous T cell lymphoma-associated antigen 1 (CTAGE1), delta like canonical Notch ligand 3 (DLL3), epidermal growth factor receptor (EGFR), epidermal growth factor receptor variant III (EGFRvIII), epithelial glycoprotein 2 (EGP2), epithelial glycoprotein 40 (EGP40), epithelial cell adhesion molecule (EPCAM), ephrin type-A receptor 2 (EPHA2), erb-b2 receptor tyrosine kinase 4 (ERBB4), fibroblast activation protein (FAP), Fc Receptor Like 5 (FCRL5), fetal acetylcholinesterase receptor (AchR), ganglioside G2 (GD2), ganglioside G3 (GD3), Glypican-3 (GPC3), EGFR family including ErbB2 (HER2), HER2 p95, IL-10Rα, IL-13Rα2, Kappa, cancer/testis antigen 2 (LAGE-1A), Lambda, Lewis-Y (LeY), L1 cell adhesion molecule (L1-CAM), melanoma antigen gene (MAGE)-A1, MAGE-A3, MAGE-A4, MAGE-A6, MAGEA10, melanoma antigen recognized by T cells 1 (MelanA or MART1), Mesothelin (MSLN), MUC, MUC16, MHC class I chain related proteins A (MICA), MHC class I chain related proteins B (MICB), neural cell adhesion molecule (NCAM), cancer/testis antigen 1 (NY-ESO-1), polysialic acid; placenta-specific 1 (PLAC1), preferentially expressed antigen in melanoma (PRAME), prostate stem cell antigen (PSCA), prostate-specific membrane antigen (PSMA), receptor tyrosine kinase-like orphan receptor 1 (ROR1), synovial sarcoma, X breakpoint 2 (SSX2), Survivin, tumor associated glycoprotein 72 (TAG72), tumor endothelial marker 1 (TEM1/CD248), tumor endothelial marker 7-related (TEM7R), trophoblast glycoprotein (TPBG), UL16-binding protein (ULBP) 1, ULBP2, ULBP3, ULBP4, ULBP5, ULBP6, vascular endothelial growth factor receptor 2 (VEGFR2), or Wilms tumor 1 (WT-1) peptide; a hinge domain selected from the group consisting of IgG1 hinge/CH2/CH3 and CD8α, and CD8α; a transmembrane domain comprising a TM domain derived from a polypeptide selected from the group consisting of CD8α; CD4, CD45, PD-1, and CD152, and a short oligo- or polypeptide linker, for example between 1, 2, 3, 4, 5, 6, 7, 8, 9, or 10 amino acids in length that links the TM domain to the intracellular signaling domain of the CAR; and one or more intracellular costimulatory signaling domains selected from the group consisting of: CD28, CD134, and CD137; and a CD3ζ primary signaling domain.

In a particular embodiment, a CAR includes an scFv that binds an alpha folate receptor (FRα), αᵥβ₆ integrin, B cell maturation antigen (BCMA), B7-H3 (CD276), B7-H6, carbonic anhydrase IX (CAIX), CD16, CD19, CD20, CD22, CD30, CD33, CD37, CD38, CD44, CD44v6, CD44v7/8, CD70, CD79a, CD79b, CD123, CD133, CD138, CD171, carcinoembryonic antigen (CEA), claudin 6, (CLDN6), claudin 18 isoform 2 (CLDN18.2), C-type lectin-like molecule-1 (CLL-1), CD2 subset 1 (CS-1), chondroitin sulfate proteoglycan 4 (CSPG4), cutaneous T cell lymphoma-associated antigen 1 (CTAGE1), delta like canonical Notch ligand 3 (DLL3), epidermal growth factor receptor (EGFR), epidermal growth factor receptor variant III (EGFRvIII), epithelial glycoprotein 2 (EGP2), epithelial glycoprotein 40 (EGP40), epithelial cell adhesion molecule (EPCAM), ephrin type-A receptor 2 (EPHA2), erb-b2 receptor tyrosine kinase 4 (ERBB4), fibroblast activation protein (FAP), Fc Receptor Like 5 (FCRL5), fetal acetylcholinesterase receptor (AchR), ganglioside G2 (GD2), ganglioside G3 (GD3), Glypican-3 (GPC3), EGFR family including ErbB2 (HER2), HER2 p95, IL-10Rα, IL-13Rα2, Kappa, cancer/testis antigen 2 (LAGE-1A), Lambda, Lewis-Y (LeY), L1 cell adhesion molecule (L1-CAM), melanoma antigen gene (MAGE)-A1, MAGE-A3, MAGE-A4, MAGE-A6, MAGEA10, melanoma antigen recognized by T cells 1 (MelanA or MART1), Mesothelin (MSLN), MUC1, MUC16, MHC class I chain related proteins A (MICA), MHC class I chain related proteins B (MICB), neural cell adhesion molecule (NCAM), cancer/testis antigen 1 (NY-ESO-1), polysialic acid; placenta-specific 1 (PLAC1), preferentially expressed antigen in melanoma (PRAME), prostate stem cell antigen (PSCA), prostate-specific membrane antigen (PSMA), receptor tyrosine kinase-like orphan receptor 1 (ROR1), synovial sarcoma, X breakpoint 2 (SSX2), Survivin, tumor associated glycoprotein 72 (TAG72), tumor endothelial marker 1 (TEM1/CD248), tumor endothelial marker 7-related (TEM7R), trophoblast glycoprotein (TPBG), UL16-binding protein (ULBP) 1, ULBP2, ULBP3, ULBP4, ULBP5, ULBP6, vascular endothelial growth factor receptor 2 (VEGFR2), or Wilms tumor 1 (WT-1) polypeptide; a hinge domain comprising a CD8α polypeptide; a CD8α transmembrane domain comprising a polypeptide linker of about 3 amino acids; one or more intracellular costimulatory signaling domains selected from the group consisting of CD28, CD134, and CD137; and a CD3ζ primary signaling domain.

### C. Dimerizing Agent Regulated Immunoreceptor Complexes (DARICs)

The immune effector cell manufacturing methods contemplated herein include modifying T cells or other immune effector cells to express one or more DARICs as contemplated herein. In various embodiments, the present disclosure provides immune effector cells (e.g., T cells) genetically engineered with vectors designed to express DARICs that redirect cytotoxicity toward tumor cells. DARIC is a drug-regulated CAR platform that i) provides control over long-term toxicity of CAR T treatment; ii) lessens persistent T cell activation and subsequent exhaustion; and iii) regulates CAR T cell activity to potentially control cytokine release syndrome. The DARIC platform separates the antigen recognition and signaling functions of a CAR into two distinct polypeptides that are further engineered to contain t small-molecule regulated dimerization domains (e.g., a human-derived FKPB12 and FRB protein). In the absence of the dimerizing drug (e.g., rapamycin or the non-immunosuppressive rapalog AP21967) the DARIC system lacks signaling activity. However, the addition of dimerizing agent drives the interaction of the two DARIC subunits, fully restoring CAR function. A discussion of DARICs is provided in Leung et al., Blood, 130 (Supplement 1):1910, 2017, WO 2019/118895 and WO 2019/118885.

In some examples, a DARIC includes: (a) a first polypeptide comprising: an FRB multimerization domain polypeptide or variant thereof, a first transmembrane domain; a first costimulatory domain; and/or a primary signaling domain; and (b) a second polypeptide comprising: one or more antibodies or antigen binding fragments thereof, an FKBP multimerization domain polypeptide or variant thereof, a second transmembrane domain; and optionally a second costimulatory domain; wherein a bridging factor promotes the formation of a polypeptide complex on the non-natural cell surface with the bridging factor associated with and disposed between the multimerization domains of the first and second polypeptides.

In some examples, a DARIC includes (a) a first polypeptide comprising: an FKBP multimerization domain polypeptide or variant thereof, a first transmembrane domain; a costimulatory domain; and/or a primary signaling domain; and (b) a second polypeptide comprising: one or more antibodies or antigen binding fragments thereof, an FRB multimerization domain polypeptide or variant thereof; a second transmembrane domain; and a second costimulatory domain; wherein a bridging factor promotes the formation of a polypeptide complex on the non-natural cell surface with the bridging factor associated with and disposed between the multimerization domains of the first and second polypeptides.

In some examples, the bridging factor is AP21967, sirolimus, everolimus, novolimus, pimecrolimus, ridaforolimus, tacrolimus, temsirolimus, umirolimus, or zotarolimus.

In some examples, the FRB multimerization domain is FRB T2098L; the FKBP multimerization domain is FKBP12; and the bridging factor is sirolimus or AP21967.

In some examples, the first transmembrane domain and the second transmembrane domain are independently selected from a polypeptide selected from the group consisting of: alpha, beta, gamma, or delta chain of the T-cell receptor, CD36, CD3E, CD37, CD3ζ, CD4, CD5, CD8α, CD9, CD 16, CD22, CD27, CD28, CD33, CD37, CD45, CD64, CD80, CD86, CD 134, CD137, CD152, CD154, AMN, and PDCD1.

In some examples, the first costimulatory domain and the second costimulatory domain are isolated from a costimulatory molecule selected from the group consisting of: Toll-like receptor 1 (TLR1), TLR2, TLR3, TLR4, TLR5, TLR6, TLR7, TLR8, TLR9, TLR10, caspase recruitment domain family member 11 (CARD 11), CD2, CD7, CD27, CD28, CD30, CD40, CD54 (ICAM), CD83, CD94, CD134 (OX40), CD137 (4-1BB), CD278 (ICOS), DNAX-Activation Protein 10 (DAP10), Linker for activation of T-cells family member 1 (LAT), SH2 Domain-Containing Leukocyte Protein Of 76 kD (SLP76), T cell receptor associated transmembrane adaptor 1 (TRAT1), TNFR2, TNFRS14, TNFRS18, TNFRS25, and zeta chain of T cell receptor associated protein kinase 70 (ZAP70).

In some examples, the primary signaling domain is isolated from a CD3ζ polypeptide.

In some examples, the one or more antibodies or antigen binding fragments thereof are independently selected is from the group consisting of: a camel Ig, a llama Ig, a shark Ig, an alpaca Ig, Ig NAR, a Fab′ fragment, a F(ab′)₂ fragment, a bispecific Fab dimer (Fab2), a trispecific Fab trimer (Fab3), an Fv, an single chain Fv protein (“scFv”), a bis-scFv, (scFv)₂, a minibody, a diabody, a triabody, a tetrabody, a disulfide stabilized Fv protein (“dsFv”), and a single-domain antibody (sdAb, a camelid VHH, nanobody).

In some examples, the one or more antibodies or antigen binding fragments thereof independently bind an antigen selected from the group consisting of: alpha folate receptor (FRα), αᵥβ₆ integrin, B cell maturation antigen (BCMA), B7-H3 (CD276), B7-H6, carbonic anhydrase IX (CAIX), CD16, CD19, CD20, CD22, CD30, CD33, CD37, CD38, CD44, CD44v6, CD44v7/8, CD70, CD79a, CD79b, CD123, CD133, CD138, CD171, carcinoembryonic antigen (CEA), claudin 6, (CLDN6), claudin 18 isoform 2 (CLDN18.2), C-type lectin-like molecule-1 (CLL-1), CD2 subset 1 (CS-1), chondroitin sulfate proteoglycan 4 (CSPG4), cutaneous T cell lymphoma-associated antigen 1 (CTAGE1), delta like canonical Notch ligand 3 (DLL3), epidermal growth factor receptor (EGFR), epidermal growth factor receptor variant Ill (EGFRvIII), epithelial glycoprotein 2 (EGP2), epithelial glycoprotein 40 (EGP40), epithelial cell adhesion molecule (EPCAM), ephrin type-A receptor 2 (EPHA2), erb-b2 receptor tyrosine kinase 4 (ERBB4), fibroblast activation protein (FAP), Fc Receptor Like 5 (FCRL5), fetal acetylcholinesterase receptor (AchR), ganglioside G2 (GD2), ganglioside G3 (GD3), Glypican-3 (GPC3), EGFR family including ErbB2 (HER2), HER2 p95, IL-10Rα, IL-13Rα2, Kappa, cancer/testis antigen 2 (LAGE-1A), Lambda, Lewis-Y (LeY), L1 cell adhesion molecule (L1-CAM), melanoma antigen gene (MAGE)-A1, MAGE-A3, MAGE-A4, MAGE-A6, MAGEA10, melanoma antigen recognized by T cells 1 (MelanA or MART1), Mesothelin (MSLN), MUC1, MUC16, MHC class I chain related proteins A (MICA), MHC class I chain related proteins B (MICB), neural cell adhesion molecule (NCAM), cancer/testis antigen 1 (NY-ESO-1), polysialic acid, placenta-specific 1 (PLAC1), preferentially expressed antigen in melanoma (PRAME), prostate stem cell antigen (PSCA), prostate-specific membrane antigen (PSMA), receptor tyrosine kinase-like orphan receptor 1 (ROR1), synovial sarcoma, X breakpoint 2 (SSX2), Survivin, tumor associated glycoprotein 72 (TAG72), tumor endothelial marker 1 (TEM1/CD248), tumor endothelial marker 7-related (TEM7R), trophoblast glycoprotein (TPBG), UL16-binding protein (ULBP) 1, ULBP2, ULBP3, ULBP4, ULBP5, ULBP6, vascular endothelial growth factor receptor 2 (VEGFR2), and Wilms tumor 1 (WT-1).

In some examples, the one or more antibodies or antigen binding fragments thereof independently bind an antigen, such as BCMA, CD33, CD123, or CLL-1.

### D. Polypeptides

The present disclosure contemplates, in part, engineered TCR, DARIC, and CAR polypeptides and fragments thereof, cells and compositions comprising the same, and vectors that express polypeptides. “Polypeptide,” “polypeptide fragment,” “peptide” and “protein” are used interchangeably, unless specified to the contrary, and according to conventional meaning, i.e., as a sequence of amino acids. Polypeptides are not limited to a specific length, e.g., they can include a full-length protein sequence or a fragment of a full length protein, and may include post-translational modifications of the polypeptide, for example, glycosylations, acetylations, phosphorylations and the like, as well as other modifications, both naturally occurring and non-naturally occurring. The polypeptides contemplated herein can include a signal (or leader) sequence at the N-terminal end of the protein, which cotranslationally or post-translationally directs transfer of the protein. Illustrative examples of suitable signal sequences useful in disclosed herein include, but are not limited to the IgG1 heavy chain signal sequence and the CD8α signal sequence. Polypeptides can be prepared using any of a variety recombinant and/or synthetic techniques. Polypeptides contemplated herein specifically encompass the CARs of the present disclosure, or sequences that have deletions from, additions to, and/or substitutions of one or more amino acid of a polypeptide as contemplated herein.

Polypeptides include “polypeptide variants.” Polypeptide variants may differ from a naturally occurring polypeptide in one or more substitutions, deletions, additions and/or insertions. Such variants may be naturally occurring or may be synthetically generated, for example, by modifying one or more of the above polypeptide sequences. For example, in particular embodiments, it may be desirable to improve the binding affinity and/or other biological properties of the engineered TCRs or CARs by introducing one or more substitutions, deletions, additions and/or insertions. In some examples, polypeptides of the disclosure include polypeptides having at least about 65%, 70%, 75%, 85%, 90%, 95%, 98%, or 99% amino acid identity thereto.

Polypeptides include “polypeptide fragments.” Polypeptide fragments refer to a polypeptide, which can be monomeric or multimeric, that has an amino-terminal deletion, a carboxyl-terminal deletion, and/or an internal deletion or substitution of a naturally-occurring or recombinantly-produced polypeptide. In certain embodiments, a polypeptide fragment can comprise an amino acid chain at least 5 to about 500 amino acids long. It will be appreciated that in certain embodiments, fragments are at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 150, 200, 250, 300, 350, 400, or 450 amino acids long.

The polypeptide may also be fused in-frame or conjugated to a linker or other sequence for ease of synthesis, purification or identification of the polypeptide (e.g., poly-His), or to enhance binding of the polypeptide to a solid support.

As noted above, polypeptides of the disclosure may be altered in various ways including amino acid substitutions, deletions, truncations, and insertions. Methods for such manipulations are generally known in the art. For example, amino acid sequence variants of a reference polypeptide can be prepared by mutations in the DNA. Methods for mutagenesis and nucleotide sequence alterations are known. See, for example, Kunkel (1985, Proc. Natl. Acad. Sci. USA. 82: 488-492), Kunkel et al., (1987, Methods in Enzymol, 154: 367-382), U.S. Pat. No. 4,873,192, Watson, J. D. et al., (Molecular Biology of the Gene, Fourth Edition, Benjamin/Cummings, Menlo Park, Calif., 1987) and the references cited therein. Guidance as to appropriate amino acid substitutions that do not affect biological activity of the protein of interest may be found in the model of Dayhoff et al., (1978) Atlas of Protein Sequence and Structure (Natl. Biomed. Res. Found., Washington, D.C.).

In certain embodiments, a variant contains conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. Modifications may be made in the structure of the polynucleotides and polypeptides of the present disclosure and still obtain a functional molecule that encodes a variant or derivative polypeptide with desirable characteristics.

Polypeptide variants further include glycosylated forms, aggregative conjugates with other molecules, and covalent conjugates with unrelated chemical moieties (e.g., pegylated molecules). Covalent variants can be prepared by linking functionalities to groups which are found in the amino acid chain or at the N- or C-terminal residue, as is known in the art. Variants also include allelic variants, species variants, and muteins. Truncations or deletions of regions which do not affect functional activity of the proteins are also variants.

In one embodiment, where expression of two or more polypeptides is desired, the polynucleotide sequences encoding them can be separated by and IRES sequence as discussed elsewhere herein. In another embodiment, two or more polypeptides can be expressed as a fusion protein that comprises one or more self-cleaving polypeptide sequences.

Polypeptides of the present disclosure include fusion polypeptides. In preferred embodiments, fusion polypeptides and polynucleotides encoding fusion polypeptides are provided. Fusion polypeptides and fusion proteins refer to a polypeptide having at least two, three, four, five, six, seven, eight, nine, or ten or more polypeptide segments. Fusion polypeptides are typically linked C-terminus to N-terminus, although they can also be linked C-terminus to C-terminus, N-terminus to N-terminus, or N-terminus to C-terminus. The polypeptides of the fusion protein can be in any order or a specified order. Fusion polypeptides or fusion proteins can also include conservatively modified variants, polymorphic variants, alleles, mutants, subsequences, and interspecies homologs, so long as the desired transcriptional activity of the fusion polypeptide is preserved. Fusion polypeptides may be produced by chemical synthetic methods or by chemical linkage between the two moieties or may generally be prepared using other standard techniques. Ligated DNA sequences comprising the fusion polypeptide are operably linked to suitable transcriptional or translational control elements as discussed elsewhere herein.

In one embodiment, a fusion partner includes a sequence that assists in expressing the protein (an expression enhancer) at higher yields than the native recombinant protein. Other fusion partners may be selected so as to increase the solubility of the protein or to enable the protein to be targeted to desired intracellular compartments or to facilitate transport of the fusion protein through the cell membrane.

Fusion polypeptides may further include a polypeptide cleavage signal between each of the polypeptide domains described herein. In addition, polypeptide site can be put into any linker peptide sequence. Exemplary polypeptide cleavage signals include polypeptide cleavage recognition sites such as protease cleavage sites, nuclease cleavage sites (e.g., rare restriction enzyme recognition sites, self-cleaving ribozyme recognition sites), and self-cleaving viral oligopeptides (see deFelipe and Ryan, 2004. Traffic, 5(8); 616-26).

Suitable protease cleavages sites and self-cleaving peptides are known (see, e.g., in Ryan et al., 1997. J. Gener. Virol. 78, 699-722; Scymczak et al. (2004) Nature Biotech. 5, 589-594). Exemplary protease cleavage sites include, but are not limited to the cleavage sites of potyvirus NIa proteases (e.g., tobacco etch virus protease), potyvirus HC proteases, potyvirus P1 (P35) proteases, byovirus NIa proteases, byovirus RNA-2-encoded proteases, aphthovirus L proteases, enterovirus 2A proteases, rhinovirus 2A proteases, picorna 3C proteases, comovirus 24K proteases, nepovirus 24K proteases, RTSV (rice tungro spherical virus) 3C-like protease, PYVF (parsnip yellow fleck virus) 3C-like protease, heparin, thrombin, factor Xa and enterokinase. Due to its high cleavage stringency, TEV (tobacco etch virus) protease cleavage sites are in one embodiment, e.g., EXXYXQ(G/S) (SEQ ID NO: 5), for example, ENLYFQG (SEQ ID NO: 6) and ENLYFQS (SEQ ID NO: 7), wherein X represents any amino acid (cleavage by TEV occurs between Q and G or Q and S).

In a particular embodiment, self-cleaving peptides include those polypeptide sequences obtained from potyvirus and cardiovirus 2A peptides, FMDV (foot-and-mouth disease virus), equine rhinitis A virus, Thosea asigna virus and porcine teschovirus.

In certain embodiments, the self-cleaving polypeptide site includes a 2A or 2A-like site, sequence or domain (Donnelly et al., 2001. J. Gen. Virol. 82:1027-1041).

### E. Polynucleotides

In particular embodiments, polynucleotides encoding one or more engineered TCR, DARIC, or CAR polypeptides contemplated herein are provided. As used herein, the terms “polynucleotide” or “nucleic acid” refers to messenger RNA (mRNA), RNA, genomic RNA (gRNA), plus strand RNA (RNA(+)), minus strand RNA (RNA(−)), genomic DNA (gDNA), complementary DNA (cDNA) or recombinant DNA. Polynucleotides include single and double stranded polynucleotides. In one example, polynucleotides of the disclosure include polynucleotides or variants having at least about 50%, 55%, 60%, 65%, 70%, 75%, 80%, 85%, 90%, 95%, 96%, 97%, 98%, 99% or 100% sequence identity to any of the reference sequences described herein (see, e.g., Sequence Listing), typically where the variant maintains at least one biological activity of the reference sequence. In various illustrative embodiments, the present disclosure contemplates, in part, polynucleotides comprising expression vectors, viral vectors, and transfer plasmids, and compositions, and cells comprising the same.

In particular embodiments, polynucleotides encode at least about 5, 10, 25, 50, 100, 150, 200, 250, 300, 350, 400, 500, 1000, 1250, 1500, 1750, or 2000 or more contiguous amino acid residues of a polypeptide of the disclosure, as well as all intermediate lengths. It will be readily understood that “intermediate lengths,” in this context, means any length between the quoted values, such as 6, 7, 8, 9, etc., 101, 102, 103, etc.; 151, 152, 153, etc.; 201, 202, 203, etc.

As used herein, the terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms include polynucleotides in which one or more nucleotides have been added or deleted, or replaced with different nucleotides compared to a reference polynucleotide. In this regard, it is well understood in the art that certain alterations inclusive of mutations, additions, deletions and substitutions can be made to a reference polynucleotide whereby the altered polynucleotide retains the biological function or activity of the reference polynucleotide.

The recitations “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Included are nucleotides and polypeptides having at least about 50%, 55%, 60%, 65%, 70%, 75%, 80%, 85%, 90%, 95%, 96%, 97%, 98%, 99% or 100% sequence identity to any of the reference sequences described herein, typically where the polypeptide variant maintains at least one biological activity of the reference polypeptide.

Terms used to describe sequence relationships between two or more polynucleotides or polypeptides include “reference sequence,” “comparison window,” “sequence identity,” “percentage of sequence identity,” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, WI, USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., Current Protocols in Molecular Biology, John Wiley & Sons Inc, 1994-1998, Chapter 15.

The polynucleotides of the present disclosure, regardless of the length of the coding sequence itself, may be combined with other DNA sequences, such as promoters and/or enhancers, untranslated regions (UTRs), Kozak sequences, polyadenylation signals, additional restriction enzyme sites, multiple cloning sites, internal ribosomal entry sites (TRES), recombinase recognition sites (e.g., LoxP, FRT, and Att sites), termination codons, transcriptional termination signals, and polynucleotides encoding self-cleaving polypeptides, epitope tags, as disclosed elsewhere herein or as known in the art, such that their overall length may vary considerably. It is therefore contemplated that a polynucleotide fragment of almost any length may be employed, with the total length for example being limited by the ease of preparation and use in the intended recombinant DNA protocol.

Polynucleotides can be prepared, manipulated and/or expressed using any of a variety of well-established techniques known and available in the art. In order to express a desired polypeptide, a nucleotide sequence encoding the polypeptide, can be inserted into appropriate vector. Examples of vectors are plasmid, autonomously replicating sequences, and transposable elements. Additional exemplary vectors include, without limitation, plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Examples of categories of animal viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40). Examples of expression vectors are pClneo vectors (Promega) for expression in mammalian cells; pLenti4/V5-DEST™, pLenti6/V5-DEST™, and pLenti6.2/V5-GW/lacZ (Invitrogen) for lentivirus-mediated gene transfer and expression in mammalian cells. In particular embodiments, the coding sequences of the chimeric proteins disclosed herein can be ligated into such expression vectors for the expression of the chimeric protein in mammalian cells.

The “control elements” or “regulatory sequences” present in an expression vector are those non-translated regions of the vector—origin of replication, selection cassettes, promoters, enhancers, translation initiation signals (Shine Dalgarno sequence or Kozak sequence) introns, a polyadenylation sequence, 5′ and 3′ untranslated regions—which interact with host cellular proteins to carry out transcription and translation. Such elements may vary in their strength and specificity. Depending on the vector system and host utilized, any number of suitable transcription and translation elements, including ubiquitous promoters and inducible promoters may be used.

In particular embodiments, a vector for use in practicing the disclosure including, but not limited to expression vectors and viral vectors, include exogenous, endogenous, or heterologous control sequences such as promoters and/or enhancers. An “endogenous” control sequence is one which is naturally linked with a given gene in the genome. An “exogenous” control sequence is one which is placed in juxtaposition to a gene by means of genetic manipulation (i.e., molecular biological techniques) such that transcription of that gene is directed by the linked enhancer/promoter. A “heterologous” control sequence is an exogenous sequence that is from a different species than the cell being genetically manipulated.

The term “promoter” as used herein refers to a recognition site of a polynucleotide (DNA or RNA) to which an RNA polymerase binds. An RNA polymerase initiates and transcribes polynucleotides operably linked to the promoter. In particular embodiments, promoters operative in mammalian cells comprise an AT-rich region located approximately 25 to 30 bases upstream from the site where transcription is initiated and/or another sequence found 70 to 80 bases upstream from the start of transcription, a CNCAAT region where N may be any nucleotide.

The term “enhancer” refers to a segment of DNA which contains sequences capable of providing enhanced transcription and in some instances can function independent of their orientation relative to another control sequence. An enhancer can function cooperatively or additively with promoters and/or other enhancer elements. The term “promoter/enhancer” refers to a segment of DNA which contains sequences capable of providing both promoter and enhancer functions.

The term “operably linked”, refers to a juxtaposition wherein the components described are in a relationship permitting them to function in their intended manner. In one embodiment, the term refers to a functional linkage between a nucleic acid expression control sequence (such as a promoter, and/or enhancer) and a second polynucleotide sequence, e.g., a polynucleotide-of-interest, wherein the expression control sequence directs transcription of the nucleic acid corresponding to the second sequence.

As used herein, the term “constitutive expression control sequence” refers to a promoter, enhancer, or promoter/enhancer that continually or continuously allows for transcription of an operably linked sequence. A constitutive expression control sequence may be a “ubiquitous” promoter, enhancer, or promoter/enhancer that allows expression in a wide variety of cell and tissue types or a “cell specific,” “cell type specific,” “cell lineage specific,” or “tissue specific” promoter, enhancer, or promoter/enhancer that allows expression in a restricted variety of cell and tissue types, respectively.

Illustrative ubiquitous expression control sequences suitable for use in particular embodiments of the disclosure include, but are not limited to, a cytomegalovirus (CMV) immediate early promoter, a viral simian virus 40 (SV40) (e.g., early or late), a Moloney murine leukemia virus (MoMLV) LTR promoter, a Rous sarcoma virus (RSV) LTR, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus (Irions et al., Nature Biotechnology 25, 1477-1482 (2007)), a Ubiquitin C promoter (UBC), a phosphoglycerate kinase-1 (PGK) promoter, a cytomegalovirus enhancer/chicken β-actin (CAG) promoter, a β-actin promoter and a myeloproliferative sarcoma virus enhancer, negative control region deleted, dl587rev primer-binding site substituted (MND) promoter (Challita et al., J Virol. 69(2):748-55 (1995)).

In a particular embodiment, it may be desirable to express a polynucleotide comprising an engineered TCR, DARIC, or CAR from a promoter that provides stable and long-term expression in T cells or other immune effector cells and at sufficient levels to redirect the T cells (or other immune effector cells) to cells expressing the target antigen. In one embodiment, the promoter is an EF1α promoter or an MND promoter.

As used herein, “conditional expression” may refer to any type of conditional expression including, but not limited to, inducible expression; repressible expression; expression in cells or tissues having a particular physiological, biological, or disease state, etc. This definition is not intended to exclude cell type or tissue specific expression. Certain embodiments of the disclosure provide conditional expression of a polynucleotide-of-interest, e.g., expression is controlled by subjecting a cell, tissue, organism, etc., to a treatment or condition that causes the polynucleotide to be expressed or that causes an increase or decrease in expression of the polynucleotide encoded by the polynucleotide-of-interest.

Illustrative examples of inducible promoters/systems include, but are not limited to, steroid-inducible promoters such as promoters for genes encoding glucocorticoid or estrogen receptors (inducible by treatment with the corresponding hormone), metallothionine promoter (inducible by treatment with various heavy metals), MX-1 promoter (inducible by interferon), the “GeneSwitch” mifepristone-regulatable system (Sirin et al., 2003, Gene, 323:67), the cumate inducible gene switch (WO 2002/088346), tetracycline-dependent regulatory systems, etc.

Conditional expression can also be achieved by using a site-specific DNA recombinase. According to certain embodiments of the disclosure the vector comprises at least one (typically two) site(s) for recombination mediated by a site-specific recombinase. As used herein, the terms “recombinase” or “site specific recombinase” include excisive or integrative proteins, enzymes, cofactors or associated proteins that are involved in recombination reactions involving one or more recombination sites (e.g., two, three, four, five, seven, ten, twelve, fifteen, twenty, thirty, fifty, etc.), which may be wild-type proteins (see Landy, Current Opinion in Biotechnology 3:699-707 (1993)), or mutants, derivatives (e.g., fusion proteins containing the recombination protein sequences or fragments thereof), fragments, and variants thereof.

Illustrative examples of recombinases suitable for use in particular embodiments of the present disclosure include, but are not limited to: Cre, Int, IHF, Xis, Flp, Fis, Hin, Gin, ΦC31, Cin, Tn3 resolvase, TndX, XerC, XerD, TnpX, Hjc, Gin, SpCCE1, and ParA.

### F. Viral Vectors

In particular embodiments, an immune effector cell (e.g., T cell) is transduced with a retroviral vector, e.g., a lentiviral vector, encoding an engineered TCR, DARIC, or CAR as contemplated herein. The transduced immune effector cell (e.g., T cell) cells elicit a stable, long-term, and persistent immune effector cell (e.g., T cell) response.

As used herein, the term “retrovirus” refers to an RNA virus that reverse transcribes its genomic RNA into a linear double-stranded DNA copy and subsequently covalently integrates its genomic DNA into a host genome. Illustrative retroviruses suitable for use in particular embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) and lentivirus.

As used herein, the term “lentivirus” refers to a group (or genus) of complex retroviruses. Illustrative lentiviruses include, but are not limited to: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In one embodiment, HIV based vector backbones (i.e., HIV cis-acting sequence elements) are used.

A vector refers to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include, e.g., replication defective retroviruses and lentiviruses.

The term “viral vector” is widely used to refer either to a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s).

The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, including LTRs that are primarily derived from a lentivirus. The term “hybrid vector” refers to a vector, LTR or other nucleic acid containing both retroviral, e.g., lentiviral, sequences and non-lentiviral viral sequences. In one embodiment, a hybrid vector refers to a vector or transfer plasmid comprising retroviral e.g., lentiviral, sequences for reverse transcription, replication, integration and/or packaging.

In particular embodiments, the terms “lentiviral vector,” “lentiviral expression vector” may be used to refer to lentiviral transfer plasmids and/or infectious lentiviral particles. Where reference is made herein to elements such as cloning sites, promoters, regulatory elements, heterologous nucleic acids, etc., it is to be understood that the sequences of these elements are present in RNA form in the lentiviral particles of the disclosure and are present in DNA form in the DNA plasmids of the disclosure.

At each end of the provirus are structures called “long terminal repeats” or “LTRs.” The term “long terminal repeat (LTR)” refers to domains of base pairs located at the ends of retroviral DNAs which, in their natural sequence context, are direct repeats and contain U3, R and U5 regions. LTRs generally provide functions fundamental to the expression of retroviral genes (e.g., promotion, initiation and polyadenylation of gene transcripts) and to viral replication. The LTR contains numerous regulatory signals including transcriptional control elements, polyadenylation signals and sequences needed for replication and integration of the viral genome. The viral LTR is divided into three regions called U3, R and U5. The U3 region contains the enhancer and promoter elements. The U5 region is the sequence between the primer binding site and the R region and contains the polyadenylation sequence. The R (repeat) region is flanked by the U3 and U5 regions. The LTR composed of U3, R and U5 regions and appears at both the 5′ and 3′ ends of the viral genome. Adjacent to the 5′ LTR are sequences necessary for reverse transcription of the genome (the tRNA primer binding site) and for efficient packaging of viral RNA into particles (the Psi site).

As used herein, the term “packaging signal” or “packaging sequence” refers to sequences located within the retroviral genome which are required for insertion of the viral RNA into the viral capsid or particle, see e.g., Clever et al., 1995. J. of Virology, Vol. 69, No. 4; pp. 2101-2109. Several retroviral vectors use the minimal packaging signal (also referred to as the psi [Ψ] sequence) needed for encapsidation of the viral genome. Thus, as used herein, the terms “packaging sequence,” “packaging signal,” “psi” and the symbol “Ψ,” are used in reference to the non-coding sequence required for encapsidation of retroviral RNA strands during viral particle formation.

In various embodiments, vectors include modified 5′ LTR and/or 3′ LTRs. Either or both of the LTR may include one or more modifications including, but not limited to, one or more deletions, insertions, or substitutions. Modifications of the 3′ LTR are often made to improve the safety of lentiviral or retroviral systems by rendering viruses replication-defective. As used herein, the term “replication-defective” refers to virus that is not capable of complete, effective replication such that infective virions are not produced (e.g., replication-defective lentiviral progeny). The term “replication-competent” refers to wild-type virus or mutant virus that is capable of replication, such that viral replication of the virus is capable of producing infective virions (e.g., replication-competent lentiviral progeny).

“Self-inactivating” (SIN) vectors refers to replication-defective vectors, e.g., retroviral or lentiviral vectors, in which the right (3′) LTR enhancer-promoter region, known as the U3 region, has been modified (e.g., by deletion or substitution) to prevent viral transcription beyond the first round of viral replication. This is because the right (3′) LTR U3 region is used as a template for the left (5′) LTR U3 region during viral replication and, thus, the viral transcript cannot be made without the U3 enhancer-promoter. In a further embodiment of the disclosure, the 3′ LTR is modified such that the U5 region is replaced, for example, with an ideal poly(A) sequence. It should be noted that modifications to the LTRs such as modifications to the 3′ LTR, the 5′ LTR, or both 3′ and 5′ LTRs, are also included in the disclosure.

An additional safety enhancement is provided by replacing the U3 region of the 5′ LTR with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. Typical promoters are able to drive high levels of transcription in a Tat-independent manner. This replacement reduces the possibility of recombination to generate replication-competent virus because there is no complete U3 sequence in the virus production system. In certain embodiments, the heterologous promoter has additional advantages in controlling the manner in which the viral genome is transcribed. For example, the heterologous promoter can be inducible, such that transcription of all or part of the viral genome will occur only when the induction factors are present. Induction factors include, but are not limited to, one or more chemical compounds or the physiological conditions such as temperature or pH, in which the host cells are cultured.

In some embodiments, viral vectors comprise a TAR element. The term “TAR” refers to the “trans-activation response” genetic element located in the R region of lentiviral (e.g., HIV) LTRs. This element interacts with the lentiviral trans-activator (tat) genetic element to enhance viral replication. However, this element is not required in embodiments wherein the U3 region of the 5′ LTR is replaced by a heterologous promoter.

The “R region” refers to the region within retroviral LTRs beginning at the start of the capping group (i.e., the start of transcription) and ending immediately prior to the start of the poly A tract. The R region is also defined as being flanked by the U3 and U5 regions. The R region plays a role during reverse transcription in permitting the transfer of nascent DNA from one end of the genome to the other.

“FLAP element” refers to a nucleic acid whose sequence includes the central polypurine tract and central termination sequences (cPPT and CTS) of a retrovirus, e.g., HIV-1 or HIV-2. Suitable FLAP elements are described in U.S. Pat. No. 6,682,907 and in Zennou, et al., 2000, Cell, 101:173. During HIV-1 reverse transcription, central initiation of the plus-strand DNA at the central polypurine tract (cPPT) and central termination at the central termination sequence (CTS) lead to the formation of a three-stranded DNA structure: the HIV-1 central DNA flap. While not wishing to be bound by any theory, the DNA flap may act as a cis-active determinant of lentiviral genome nuclear import and/or may increase the titer of the virus. In particular embodiments, the retroviral or lentiviral vector backbones comprise one or more FLAP elements upstream or downstream of the heterologous genes of interest in the vectors. For example, in particular embodiments a transfer plasmid includes a FLAP element. In one embodiment, a vector of the disclosure comprises a FLAP element isolated from HIV-1.

In one embodiment, retroviral or lentiviral transfer vectors comprise one or more export elements. The term “export element” refers to a cis-acting post-transcriptional regulatory element which regulates the transport of an RNA transcript from the nucleus to the cytoplasm of a cell. Examples of RNA export elements include, but are not limited to, the human immunodeficiency virus (HIV) rev response element (RRE) (see e.g., Cullen et al., 1991. J. Virol. 65: 1053; and Cullen et al., 1991. Cell 58: 423), and the hepatitis B virus post-transcriptional regulatory element (HPRE). Generally, the RNA export element is placed within the 3′ UTR of a gene, and can be inserted as one or multiple copies.

In particular embodiments, expression of heterologous sequences in viral vectors is increased by incorporating posttranscriptional regulatory elements, efficient polyadenylation sites, and optionally, transcription termination signals into the vectors. A variety of posttranscriptional regulatory elements can increase expression of a heterologous nucleic acid at the protein, e.g., woodchuck hepatitis virus posttranscriptional regulatory element (WPRE; Zufferey et al., 1999, J. Virol., 73:2886); the posttranscriptional regulatory element present in hepatitis B virus (HPRE) (Huang et al., Mol. Cell. Biol., 5:3864); and the like (Liu et al., 1995, Genes Dev., 9:1766). In particular embodiments, vectors of the disclosure comprise a posttranscriptional regulatory element such as a WPRE or HPRE.

In particular embodiments, vectors lack or do not comprise a posttranscriptional regulatory element such as a WPRE or HPRE because in some instances these elements increase the risk of cellular transformation and/or do not substantially or significantly increase the amount of mRNA transcript or increase mRNA stability. Therefore, in some embodiments, vectors lack or do not include a WPRE or HPRE as an added safety measure.

Elements directing the efficient termination and polyadenylation of the heterologous nucleic acid transcripts increases heterologous gene expression. Transcription termination signals are generally found downstream of the polyadenylation signal. In particular embodiments, vectors comprise a polyadenylation sequence 3′ of a polynucleotide encoding a polypeptide to be expressed. The term “polyA site” or “polyA sequence” as used herein denotes a DNA sequence which directs both the termination and polyadenylation of the nascent RNA transcript by RNA polymerase II. Polyadenylation sequences can promote mRNA stability by addition of a polyA tail to the 3′ end of the coding sequence and thus, contribute to increased translational efficiency. Efficient polyadenylation of the recombinant transcript is desirable as transcripts lacking a poly A tail are unstable and are rapidly degraded. Illustrative examples of polyA signals that can be used in a vector of the disclosure, includes an ideal polyA sequence (e.g., AATAAA, ATTAAA, AGTAAA), a bovine growth hormone polyA sequence (BGHpA), a rabbit β-globin polyA sequence (rβgpA), or another suitable heterologous or endogenous polyA sequence known in the art.

In various embodiments, the vectors of the disclosure comprise a promoter operably linked to a polynucleotide encoding an engineered TCR, DARIC, or CAR polypeptide. The vectors may have one or more LTRs, wherein either LTR comprises one or more modifications, such as one or more nucleotide substitutions, additions, or deletions. The vectors may further comprise one of more accessory elements to increase transduction efficiency (e.g., a cPPT/FLAP), viral packaging (e.g., a Psi (Ψ) packaging signal, RRE), and/or other elements that increase therapeutic gene expression (e.g., poly (A) sequences), and may optionally comprise a WPRE or HPRE. The skilled artisan would appreciate that many other different embodiments can be fashioned from the existing embodiments of the disclosure.

A “host cell” includes cells transfected, infected, or transduced in vivo, ex vivo, or in vitro with a recombinant vector or a polynucleotide of the disclosure. Host cells may include packaging cells, producer cells, and cells infected with viral vectors. In particular embodiments, host cells infected with viral vector of the disclosure are administered to a subject in need of therapy. In certain embodiments, the term “target cell” is used interchangeably with host cell and refers to transfected, infected, or transduced cells of a desired cell type. In preferred embodiments, the target cell is an immune effector cell (e.g., T cell).

Large scale viral particle production is often necessary to achieve a reasonable viral titer. Viral particles are produced by transfecting a transfer vector into a packaging cell line that comprises viral structural and/or accessory genes, e.g., gag, pol, env, tat, rev, vif, vpr, vpu, vpx, or nef genes or other retroviral genes.

As used herein, the term “packaging vector” refers to an expression vector or viral vector that lacks a packaging signal and comprises a polynucleotide encoding one, two, three, four or more viral structural and/or accessory genes. Typically, the packaging vectors are included in a packaging cell, and are introduced into the cell via transfection, transduction or infection. Methods for transfection, transduction or infection are known. A retroviral/lentiviral transfer vector of the present disclosure can be introduced into a packaging cell line, via transfection, transduction or infection, to generate a producer cell or cell line. The packaging vectors of the present disclosure can be introduced into human cells or cell lines by standard methods including, e.g., calcium phosphate transfection, lipofection or electroporation. In some embodiments, the packaging vectors are introduced into the cells together with a dominant selectable marker, such as neomycin, hygromycin, puromycin, blastocidin, zeocin, thymidine kinase, DHFR, Gln synthetase or ADA, followed by selection in the presence of the appropriate drug and isolation of clones. A selectable marker gene can be linked physically to genes encoding by the packaging vector, e.g., by IRES or self-cleaving viral peptides.

Viral envelope proteins (env) determine the range of host cells which can ultimately be infected and transformed by recombinant retroviruses generated from the cell lines. In the case of lentiviruses, such as HIV-1, HIV-2, SIV, FIV and EIV, the env proteins include gp41 and gp120. In some examples, the viral env proteins expressed by packaging cells of the disclosure are encoded on a separate vector from the viral gag and pol genes, as has been previously described.

Illustrative examples of retroviral-derived env genes which can be employed in the disclosure include, but are not limited to: MLV envelopes, 10A1 envelope, BAEV, FeLV-B, RD114, SSAV, Ebola, Sendai, FPV (Fowl plague virus), and influenza virus envelopes. Similarly, genes encoding envelopes from RNA viruses (e.g., RNA virus families of Picornaviridae, Calciviridae, Astroviridae, Togaviridae, Flaviviridae, Coronaviridae, Paramyxoviridae, Rhabdoviridae, Filoviridae, Orthomyxoviridae, Bunyaviridae, Arenaviridae, Reoviridae, Birnaviridae, Retroviridae) as well as from the DNA viruses (families of Hepadnaviridae, Circoviridae, Parvoviridae, Papovaviridae, Adenoviridae, Herpesviridae, Poxyiridae, and Iridoviridae) may be utilized. Representative examples include FeLV, VEE, HFVW, WDSV, SFV, Rabies, ALV, BIV, BLV, EBV, CAEV, SNV, ChTLV, STLV, MPMV, SMRV, RAV, FuSV, MH2, AEV, AMV, CT10, and EIAV.

In other embodiments, envelope proteins for pseudotyping a virus of present disclosure include, but are not limited to any of the following virus: Influenza A such as HINI, H1N2, H3N2 and H5N1 (bird flu), Influenza B, Influenza C virus, Hepatitis A virus, Hepatitis B virus, Hepatitis C virus, Hepatitis D virus, Hepatitis E virus, Rotavirus, any virus of the Norwalk virus group, enteric adenoviruses, parvovirus, Dengue fever virus, Monkey pox, Mononegavirales, Lyssavirus such as rabies virus, Lagos bat virus, Mokola virus, Duvenhage virus, European bat virus 1 & 2 and Australian bat virus, Ephemerovirus, Vesiculovirus, Vesicular Stomatitis Virus (VSV), Herpesviruses such as Herpes simplex virus types 1 and 2, varicella zoster, cytomegalovirus, Epstein-Bar virus (EBV), human herpesviruses (HHV), human herpesvirus type 6 and 8, Human immunodeficiency virus (HIV), papilloma virus, murine gammaherpesvirus, Arenaviruses such as Argentine hemorrhagic fever virus, Bolivian hemorrhagic fever virus, Sabia-associated hemorrhagic fever virus, Venezuelan hemorrhagic fever virus, Lassa fever virus, Machupo virus, Lymphocytic choriomeningitis virus (LCMV), Bunyaviridiae such as Crimean-Congo hemorrhagic fever virus, Hantavirus, hemorrhagic fever with renal syndrome causing virus, Rift Valley fever virus, Filoviridae (filovirus) including Ebola hemorrhagic fever and Marburg hemorrhagic fever, Flaviviridae including Kaysanur Forest disease virus, Omsk hemorrhagic fever virus, Tick-borne encephalitis causing virus and Paramyxoviridae such as Hendra virus and Nipah virus, variola major and variola minor (smallpox), alphaviruses such as Venezuelan equine encephalitis virus, eastern equine encephalitis virus, western equine encephalitis virus, SARS-associated coronavirus (SARS-CoV), West Nile virus, any encephaliltis causing virus.

In one embodiment, the disclosure provides packaging cells which produce recombinant retrovirus, e.g., lentivirus, pseudotyped with the VSV-G glycoprotein.

The terms “pseudotype” or “pseudotyping” as used herein, refer to a virus whose viral envelope proteins have been substituted with those of another virus possessing preferable characteristics. For example, HIV can be pseudotyped with vesicular stomatitis virus G-protein (VSV-G) envelope proteins, which allows HIV to infect a wider range of cells because HIV envelope proteins (encoded by the env gene) normally target the virus to CD4+ presenting cells. In one embodiment, lentiviral envelope proteins are pseudotyped with VSV-G. In one embodiment, packaging cells are provided which produce recombinant retrovirus, e.g., lentivirus, pseudotyped with the VSV-G envelope glycoprotein.

As used herein, the term “packaging cell lines” is used in reference to cell lines that do not contain a packaging signal, but do stably or transiently express viral structural proteins and replication enzymes (e.g., gag, pol and env) which are necessary for the correct packaging of viral particles. Any suitable cell line can be employed to prepare packaging cells of the disclosure. Generally, the cells are mammalian cells. In a particular embodiment, the cells used to produce the packaging cell line are human cells. Suitable cell lines which can be used include, for example, CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells. In preferred embodiments, the packaging cells are 293 cells, 293T cells, or A549 cells.

As used herein, the term “producer cell line” refers to a cell line which is capable of producing recombinant retroviral particles, comprising a packaging cell line and a transfer vector construct comprising a packaging signal. The production of infectious viral particles and viral stock solutions may be carried out using conventional techniques. Methods of preparing viral stock solutions are illustrated by, e.g., Y. Soneoka et al. (1995) Nucl. Acids Res. 23:628-633, and N. R. Landau et al. (1992) J. Virol. 66:5110-5113. Infectious virus particles may be collected from the packaging cells using conventional techniques. For example, the infectious particles can be collected by cell lysis, or collection of the supernatant of the cell culture, as is known in the art. Optionally, the collected virus particles may be purified if desired. Suitable purification techniques are known.

The delivery of a gene(s) or other polynucleotide sequence using a retroviral or lentiviral vector by means of viral infection rather than by transfection is referred to as “transduction.” In one embodiment, retroviral vectors are transduced into a cell through infection and provirus integration. In certain embodiments, a target cell, e.g., a T cell, is “transduced” if it comprises a gene or other polynucleotide sequence delivered to the cell by infection using a viral or retroviral vector. In particular embodiments, a transduced cell comprises one or more genes or other polynucleotide sequences delivered by a retroviral or lentiviral vector in its cellular genome.

In particular embodiments, immune effector cells (e.g., T cells) transduced with viral vector of the disclosure that expresses one or more polypeptides, are administered to a subject to treat and/or prevent a malignancy. Other methods relating to the use of viral vectors in gene therapy, which may be utilized according to certain embodiments of the present disclosure, can be found in, e.g., Kay, M. A. (1997) Chest 111(6 Supp.):138S-142S; Ferry, N. and Heard, J. M. (1998) Hum. Gene Ther. 9:1975-81; Shiratory, Y. et al. (1999) Liver 19:265-74; Oka, K. et al. (2000) Curr. Opin. Lipidol. 11:179-86; Thule, P. M. and Liu, J. M. (2000) Gene Ther. 7:1744-52; Yang, N. S. (1992) Crit. Rev. Biotechnol. 12:335-56; Alt, M. (1995) J. Hepatol. 23:746-58; Brody, S. L. and Crystal, R. G. (1994) Ann. N.Y. Acad. Sci. 716:90-101; Strayer, D. S. (1999) Expert Opin. Investig. Drugs 8:2159-2172; Smith-Arica, J. R. and Bartlett, J. S. (2001) Curr. Cardiol. Rep. 3:43-49; and Lee, H. C. et al. (2000) Nature 408:483-8.

### G. Compositions and Formulations

The compositions contemplated herein can include one or more polypeptides, polynucleotides, vectors comprising same, and immune effector cell (e.g., T cell) compositions, as contemplated herein. In some embodiments, the composition includes a population of immune effector cells generated using the disclosed methods. Such a population of immune effector cells can, in some examples, include a vector comprising an engineered antigen receptor (such as a TCR, DARIC, or CAR), and thus have increased respiratory capacity and/or mitochondrial mass as described herein. In certain embodiments, the immune effector cells are TILs. In various embodiments, a composition comprising a population of immune effector cells comprising a vector comprising an engineered antigen receptor (such as a TCR, DARIC, or CAR), produced using the culturing methods provided herein is provided. Such a composition can further include a physiologically acceptable excipient. In some examples, such a composition includes DMSO. In some examples, such a composition is frozen.

Compositions include, but are not limited to pharmaceutical compositions. A “pharmaceutical composition” refers to a composition formulated in pharmaceutically-acceptable or physiologically-acceptable solutions for administration to a cell or an animal, either alone, or in combination with one or more other modalities of therapy. It will also be understood that, if desired, the compositions of the disclosure may be administered in combination with other agents as well, such as, e.g., cytokines, growth factors, hormones, small molecules, chemotherapeutics, pro-drugs, drugs, antibodies, or other various pharmaceutically-active agents. There is virtually no limit to other components that may also be included in the compositions, provided that the additional agents do not adversely affect the ability of the composition to deliver the intended therapy.

Pharmaceutically acceptable carriers include compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations.

In particular embodiments, compositions of the present disclosure include an amount modified immune effector cell (e.g., T cell) manufactured by the methods contemplated herein. In some embodiments, the pharmaceutical T cell compositions comprises potent immune effector cells (e.g., T cells) having one or more of, or all of the following markers: CD62L, CD127, CD197, and CD38.

It can generally be stated that a pharmaceutical composition comprising the immune effector cells (e.g., T cells) manufactured by the methods contemplated herein may be administered at a dosage of 10² to 10¹⁰ cells/kg body weight, 10⁵ to 10⁹ cells/kg body weight, 10⁵ to 10⁸ cells/kg body weight, 10⁵ to 10⁷ cells/kg body weight, 10⁷ to 10⁹ cells/kg body weight, or 10⁷ to 10⁸ cells/kg body weight, including all integer values within those ranges. The number of cells will depend upon the ultimate use for which the composition is intended as will the type of cells included therein. For uses provided herein, the cells are generally in a volume of a liter or less, can be 500 mL or less, even 250 mL or 100 mL or less. Hence the density of the desired cells is typically greater than 10⁶ cells/ml and generally is greater than 10⁷ cells/ml, generally 10⁸ cells/ml or greater. The clinically relevant number of immune cells can be apportioned into multiple infusions that cumulatively equal or exceed 10⁵, 10⁶, 10⁷, 10⁸, 10⁹, 10¹⁰, 10¹¹, or 10¹² cells. In some aspects of the present disclosure, particularly since all the infused cells will be redirected to a particular target antigen (e.g., BCMA, CD33, CD123, or CLL1), lower numbers of cells, in the range of 10⁶/kilogram (10⁶-10¹¹ per patient) may be administered. T cells modified to express an engineered TCR, DARIC, or CAR may be administered multiple times at dosages within these ranges. The cells may be allogeneic, syngeneic, xenogeneic, or autologous to the patient undergoing therapy. If desired, the treatment may also include administration of mitogens (e.g., PHA) or lymphokines, cytokines, and/or chemokines (e.g., IFN-γ, IL-2, IL-7, IL-15, IL-12, TNF-alpha, IL-18, and TNF-beta, GM-CSF, IL-4, IL-13, Flt3-L, RANTES, MIP1α, etc.) as described herein to enhance engraftment and function of infused immune effector cell (e.g., T cell).

Generally, compositions comprising the immune effector cells (e.g., T cells) activated and expanded as described herein may be utilized in the treatment and prevention of diseases that arise in individuals who are immunocompromised. In particular, compositions comprising the modified immune effector cells (e.g., T cells) manufactured by the methods contemplated herein are used in the treatment of cancer. The modified immune effector cells (e.g., T cells) of the present disclosure may be administered either alone, or as a pharmaceutical composition in combination with carriers, diluents, excipients, and/or with other components such as IL-2, IL-7, and/or IL-15 or other cytokines or cell populations. In particular embodiments, pharmaceutical compositions contemplated herein comprise an amount of genetically modified immune effector cells (e.g., T cells), in combination with one or more pharmaceutically or physiologically acceptable carriers, diluents or excipients.

Pharmaceutical compositions comprising modified immune effector cells (e.g., T cells) contemplated herein may further include buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure can be formulated for parenteral administration, e.g., intravascular (intravenous or intraarterial), intraperitoneal or intramuscular administration.

The liquid pharmaceutical compositions, whether they be solutions, suspensions or other like form, may include one or more of the following: sterile diluents such as water for injection, saline solution, for example physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. An injectable pharmaceutical composition is in some examples sterile.

In a particular embodiment, compositions contemplated herein include an effective amount of an expanded modified immune effector cell (e.g., T cell) composition, alone or in combination with one or more therapeutic agents. Thus, the immune effector cell (e.g., T cell) compositions may be administered alone or in combination with other known cancer treatments, such as radiation therapy, chemotherapy, transplantation, immunotherapy, hormone therapy, photodynamic therapy, etc. The compositions may also be administered in combination with antibiotics. Such therapeutic agents may be accepted in the art as a standard treatment for a particular disease state as described herein, such as a particular cancer. Exemplary therapeutic agents contemplated include cytokines, growth factors, steroids, NSAIDs, DMARDs, anti-inflammatories, chemotherapeutics, radiotherapeutics, therapeutic antibodies, or other active and ancillary agents.

In certain embodiments, compositions including immune effector cells (e.g., T cells) contemplated herein may be administered in conjunction with any number of chemotherapeutic agents. Illustrative examples of chemotherapeutic agents include alkylating agents such as thiotepa and cyclophosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine resume; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2, 2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.J.) and doxetaxel (TAXOTERE®, Rhne-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylomithine (DMFO); retinoic acid derivatives such as Targretin™ (bexarotene), Panretin™ (alitretinoin); ONTAK™ (denileukin diftitox); esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

A variety of other therapeutic agents may be used in conjunction with the immune effector cell (e.g., T cell) compositions described herein. In one embodiment, the composition containing immune effector cells (e.g., T cells) is administered with an anti-inflammatory agent. Anti-inflammatory agents or drugs include, but are not limited to, steroids and glucocorticoids (including betamethasone, budesonide, dexamethasone, hydrocortisone acetate, hydrocortisone, hydrocortisone, methylprednisolone, prednisolone, prednisone, triamcinolone), nonsteroidal anti-inflammatory drugs (NSAIDS) including aspirin, ibuprofen, naproxen, methotrexate, sulfasalazine, leflunomide, anti-TNF medications, cyclophosphamide and mycophenolate.

Other exemplary NSAIDs are chosen from the group consisting of ibuprofen, naproxen, naproxen sodium, Cox-2 inhibitors such as VIOXX® (rofecoxib) and CELEBREX® (celecoxib), and sialylates. Exemplary analgesics are chosen from the group consisting of acetaminophen, oxycodone, tramadol of proporxyphene hydrochloride. Exemplary glucocorticoids are chosen from the group consisting of cortisone, dexamethasone, hydrocortisone, methylprednisolone, prednisolone, or prednisone. Exemplary biological response modifiers include molecules directed against cell surface markers (e.g., CD4, CD5, etc.), cytokine inhibitors, such as the TNF antagonists (e.g., etanercept (ENBREL®), adalimumab (HUMIRA®) and infliximab (REMICADE®), chemokine inhibitors and adhesion molecule inhibitors. The biological response modifiers include monoclonal antibodies as well as recombinant forms of molecules. Exemplary DMARDs include azathioprine, cyclophosphamide, cyclosporine, methotrexate, penicillamine, leflunomide, sulfasalazine, hydroxychloroquine, Gold (oral (auranofin) and intramuscular) and minocycline.

Illustrative examples of therapeutic antibodies suitable for combination with the TCR, CAR, or DARIC modified immune effector cells (e.g., T cells) contemplated herein, include but are not limited to, abagovomab, adecatumumab, afutuzumab, alemtuzumab, altumomab, amatuximab, anatumomab, arcitumomab, bavituximab, bectumomab, bevacizumab, bivatuzumab, blinatumomab, brentuximab, cantuzumab, catumaxomab, cetuximab, citatuzumab, cixutumumab, clivatuzumab, conatumumab, daratumumab, drozitumab, duligotumab, dusigitumab, detumomab, dacetuzumab, dalotuzumab, ecromeximab, elotuzumab, ensituximab, ertumaxomab, etaracizumab, farietuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab, iratumumab, labetuzumab, lexatumumab, lintuzumab, lorvotuzumab, lucatumumab, mapatumumab, matuzumab, milatuzumab, minretumomab, mitumomab, moxetumomab, narnatumab, naptumomab, necitumumab, nimotuzumab, nofetumomab, ocaratuzumab, ofatumumab, olaratumab, onartuzumab, oportuzumab, oregovomab, panitumumab, parsatuzumab, patritumab, pemtumomab, pertuzumab, pintumomab, pritumumab, racotumomab, radretumab, rilotumumab, rituximab, robatumumab, satumomab, sibrotuzumab, siltuximab, simtuzumab, solitomab, tacatuzumab, taplitumomab, tenatumomab, teprotumumab, tigatuzumab, tositumomab, trastuzumab, tucotuzumab, ublituximab, veltuzumab, vorsetuzumab, votumumab, zalutumumab, CC49 and 3F8.

In one example, the TCR, CAR, or DARIC modified immune effector cells (e.g., T cells) are administered in conjunction with a biologic, such as a PD-1 antagonist; a PD-L1 antagonist; a CTLA4 antagonist; a T cell agonist; or combinations thereof. In one example, the PD-1 antagonist, PD-L1 antagonist, CTLA4 antagonist, and T cell agonist, are mAbs, such as Atezolizumab, MPDL3280A, BNS-936558 (Nivolumab), Pembrolizumab, Pidilizumab, CT011, AMP-224, AMP-514, MEDI-0680, BMS-936559, BMS935559, MEDI-4736, MPDL-3280A, MSB-0010718C, MGA-271, Indoximod, Epacadostat, BMS-986016, MEDI-4736, MEDI-4737, MK-4166, BMS-663513, PF-05082566 (PF-2566), Lirilumab, or Durvalumab. The cells can be administered before, during or after the other agents. Exemplary T cell agonists include agonists of 4-1BB, agonists of OX40, and agonists GITR, such as a mAb, aptamer, or ligand for these receptors. Exemplary agonists of 4-1BB that can be administered mAbs, such as PF-05082566 (utomilumab) or BMS-663513 (Urelumab), or a ligand (e.g., 4-1BBL or SA-4-1BBL). Exemplary agonists of OX40 that can be administered include a mAb (e.g., PF-04518600, MEDI6469, MEDI0562, MEDI6383, MOXR0916, BMS 986178, or GSK3174998), or a ligand (e.g., OX40L). Exemplary agonists of GITR that can be administered include a GITR agonist mAb, such as DTA-1, TRX518, MK-4166, MK-1248, AMG 228, INCAGN01876, GWN323 (from Novartis), CK-302 (from Checkpoint Therapeutics) or BMS-986156. Exemplary agonists of GITR that can be administered include a GITR ligand (GITRL), such as a natural GITRL or a multivalent GITR ligand fusion protein, such as MEDI1873.

In certain embodiments, the compositions containing the disclosed immune effector cells (e.g., T cells) described herein are administered in conjunction with a cytokine. By “cytokine” as used herein is meant a generic term for proteins released by one cell population that act on another cell as intercellular mediators. Examples of such cytokines are lymphokines, monokines, chemokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1alpha, IL-2, IL-3, IL-4, IL-5, IL-6, IL-7, IL-8, IL-9, IL-10, IL-11, IL-12; IL-15, a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of the native sequence cytokines.

In some examples, the subject administered the compositions containing the disclosed immune effector cells (e.g., T cells) described herein are administered is also administered an effective amount of nonmyeloablative chemotherapy or radiotherapy. For example, the subject may receive an effective amount of nonmyeloablative chemotherapy, such as administration of one or more of cisplatin, fludarabine, idarubicin, melphalan, ara-C, 2-chlorodeoxyadenosine, antithymocyte globulin, and cyclophosphamide (such as 10 to 50 mg/kg body weight). In some examples, the subject receives an effective amount of solid tumor irradiation, thymic irradiation, or total body irradiation (e.g., 2 Gy), or combinations thereof.

In particular embodiments, a composition includes TCR, CAR, or DARIC modified immune effector cells (e.g., T cells) contemplated herein express one or more of the following markers: CD3, CD4, CD8, CD28, CD45RA, CD45RO, CD62, CD127, and HLA-DR can be further isolated by positive or negative selection techniques. In one embodiment, a composition comprises a specific subpopulation of T cells, expressing one or more of the markers selected from the group consisting of CD62L, CCR7, CD28, CD27, CD122, CD127, CD197; and CD38 or CD62L, CD127, CD197, and CD38, is further isolated by positive or negative selection techniques. In various embodiments, compositions do not express or do not substantially express one or more of the following markers: CD57, CD244, CD160, PD-1, CTLA4, TIM3, and LAG3.

Also provided are containers that include a composition disclosed herein, such as one containing immune effector cells (e.g., T cells) generated using the methods provided herein. In some embodiments, the container is a syringe. In some examples, the syringe includes a needle. The plunger in a syringe can have a stopper to prevent the plunger from being accidentally removed during aspiration. Disposable syringes generally contain a single dose of immune effector cells (e.g., T cells). The syringe can have a tip cap to seal the tip prior to attachment of a needle. In non-limiting examples, the tip cap is made of rubber, such as a butyl rubber.

In some embodiments, the container is a vial. In some examples, the vial is made of glass, such as a colorless glass, for example borosilicate. In other examples, the vial is made of plastic. The vial can include a stopper, such as a rubber stopper, or a cap, such as cap adapted to enable insertion of a syringe. In some examples, the vial includes a single dose of the composition. In other examples, the vial includes multiples doses of the composition, such as 2, 3, 4, 5, 6, 7, 8, 9 or 10 or more doses of the composition. Generally, the vial is sterilized prior to adding the composition.

Also provided are kits that include immune effector cells (e.g., T cells) generated using the methods provided herein, for example a container of the immune effector cells (e.g., T cells). Such kits can include additional reagents, such as one or more anti-cancer reagents, such as a chemotherapeutic, therapeutic antibody, or combination thereof. Examples of such compounds are provided herein. In some examples, in a kit, the immune effector cells (e.g., T cells) and anti-cancer reagents are present in separate containers. The compositions can be in a suspension, such as in PBS or other pharmaceutically acceptable carrier. Alternatively, the compositions can be in a dried or powered form, such as lyophilized or freeze dried, which can then be reconstituted by an end user (for example with PBS or other pharmaceutically acceptable carrier). In some examples the containers can include a pharmaceutically acceptable carrier, such as PBS, or the pharmaceutically acceptable carrier, such as PBS, can be in a separate container (for example if the compositions are freeze-dried or lyophilized). In some examples, the containers in the kit further include one or more stabilizers. In some examples, the kits also include a device that permits administration of the composition to a subject. Examples of such devices include a syringe. A kit can be packaged (for example, in the same box) with a leaflet including details of the composition, such as instructions for administration and/or details of the immune effector cells (e.g., T cells) within the composition.

### VI. Target Cells and Antigens

The present disclosure contemplates, in part, genetically modified immune effector cells redirected to a target cell, e.g., a tumor or cancer cell, and that comprise engineered TCRs, DARICs, or CARs having a binding domain that binds to target antigens on the cells. Cancer cells can also spread to other parts of the body through the blood and lymph systems. There are several main types of cancer. Carcinoma is a cancer that begins in the skin or in tissues that line or cover internal organs. Sarcoma is a cancer that begins in bone, cartilage, fat, muscle, blood vessels, or other connective or supportive tissue. Leukemia is a cancer that starts in blood-forming tissue such as the bone marrow, and causes large numbers of abnormal blood cells to be produced and enter the blood. Lymphoma and multiple myeloma are cancers that begin in the cells of the immune system. Central nervous system cancers are cancers that begin in the tissues of the brain and spinal cord.

In one embodiment, the target cell expresses an antigen, e.g., target antigen, which is not substantially found on the surface of other normal (desired) cells. In one embodiment, the target cell is a pancreatic parenchymal cell, pancreatic duct cell, hepatic cell, cardiac muscle cell, skeletal muscle cell, osteoblast, skeletal myoblast, neuron, vascular endothelial cell, pigment cell, smooth muscle cell, glial cell, fat cell, bone cell, chondrocyte, pancreatic islet cell, CNS cell, PNS cell, liver cell, adipose cell, renal cell, lung cell, skin cell, ovary cell, follicular cell, epithelial cell, immune cell, or an endothelial cell.

In certain embodiments, the target cell is part of a pancreatic tissue, neural tissue, cardiac tissue, bone marrow, muscle tissue, bone tissue, skin tissue, liver tissue, hair follicles, vascular tissue, adipose tissue, lung tissue, and kidney tissue.

In a particular embodiment, the target cell is a tumor cell. In another particular embodiment, the target cell is a cancer cell, such as a cell in a patient with cancer. Exemplary cells that can be killed with the disclosed methods include cells of the following tumors: a liquid tumor such as a leukemia, including acute leukemia (such as acute lymphocytic leukemia, acute myelocytic leukemia, and myeloblastic, promyelocytic, myelomonocytic, monocytic and erythroleukemia), chronic leukemias (such as chronic myelocytic (granulocytic) leukemia and chronic lymphocytic leukemia), polycythemia vera, lymphoma, Hodgkin's disease, non-Hodgkin's lymphoma, multiple myeloma, Waldenstrom's macroglobulinemia, heavy chain disease).

In another embodiment, the cell is a solid tumor cell, such as sarcomas and carcinomas, fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, pancreatic cancer, breast cancer, ovarian cancer, prostate cancer, hepatocellular carcinoma, lung cancer, colorectal cancer, squamous cell carcinoma, basal cell carcinoma, adenocarcinoma (for example adenocarcinoma of the pancreas, colon, ovary, lung, breast, stomach, prostate, cervix, or esophagus), sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, bladder carcinoma, CNS tumors (such as a glioma, astrocytoma, medulloblastoma, craniopharyogioma, ependymoma, pinealoma, hemangioblastoma, acoustic neuroma, oligodendroglioma, menangioma, melanoma, neuroblastoma and retinoblastoma).

In one embodiment, the cancer is selected from the group consisting of. Wilms' tumor, Ewing sarcoma, a neuroendocrine tumor, a glioblastoma, a neuroblastoma, a melanoma, skin cancer, breast cancer, colon cancer, rectal cancer, prostate cancer, liver cancer, renal cancer, pancreatic cancer, lung cancer, biliary cancer, cervical cancer, endometrial cancer, esophageal cancer, gastric cancer, head and neck cancer, medullary thyroid carcinoma, ovarian cancer, glioma, lymphoma, leukemia, myeloma, acute lymphoblastic leukemia, acute myelogenous leukemia, chronic lymphocytic leukemia, chronic myelogenous leukemia, Hodgkin's lymphoma, non-Hodgkin's lymphoma, and urinary bladder cancer.

In one embodiment, the target cell is a malignant cell of the liver, pancreas, lung, breast, bladder, brain, bone, thyroid, kidney, skin, and hematopoietic system. In another embodiment, the target cell is a cell in a liver cancer, pancreatic cancer, lung cancer, breast cancer, bladder cancer, brain cancer, bone cancer, thyroid cancer, kidney cancer, skin cancer, or hematological cancer.

In one example, the target cell is a hematological malignancy, such as multiple myeloma (MM), chronic lymphocytic leukemia (CLL), acute myeloid leukemia (AML), or non-Hodgkin's lymphoma (NHL) cell. In some such examples, the engineered antigen receptor comprises an extracellular binding domain that binds BCMA, CD33, CD123, or CLL1.

In one embodiment, the target cell is a cancer cell infected by a virus, including but not limited to CMV, HPV, hepatitis C virus (HCV) and EBV. Thus, methods for treating a cancer associated with or caused by a virus (such as CMV, HPV, HCV, and EBV) are provided.

In one embodiment, the target antigen is an epitope of alpha folate receptor (FRα), αᵥβ₆ integrin, B cell maturation antigen (BCMA), B7-H3 (CD276), B7-H6, carbonic anhydrase IX (CAIX), CD16, CD19, CD20, CD22, CD30, CD33, CD37, CD38, CD44, CD44v6, CD44v7/8, CD70, CD79a, CD79b, CD123, CD133, CD138, CD171, carcinoembryonic antigen (CEA), claudin 6, (CLDN6), claudin 18 isoform 2 (CLDN18.2), C-type lectin-like molecule-1 (CLL-A), CD2 subset 1 (CS-1), chondroitin sulfate proteoglycan 4 (CSPG4), cutaneous T cell lymphoma-associated antigen 1 (CTAGE1), delta like canonical Notch ligand 3 (DLL3), epidermal growth factor receptor (EGFR), epidermal growth factor receptor variant III (EGFRvIII), epithelial glycoprotein 2 (EGP2), epithelial glycoprotein 40 (EGP40), epithelial cell adhesion molecule (EPCAM), ephrin type-A receptor 2 (EPHA2), erb-b2 receptor tyrosine kinase 4 (ERBB4), fibroblast activation protein (FAP), Fc Receptor Like 5 (FCRL5), fetal acetylcholinesterase receptor (AchR), ganglioside G2 (GD2), ganglioside G3 (GD3), Glypican-3 (GPC3), EGFR family including ErbB2 (HER2), HER2 p95, IL-10Rα, IL-13Rα2, Kappa, cancer/testis antigen 2 (LAGE-1A), Lambda, Lewis-Y (LeY), L1 cell adhesion molecule (L1-CAM), melanoma antigen gene (MAGE)-A1, MAGE-A3, MAGE-A4, MAGE-A6, MAGEA10, melanoma antigen recognized by T cells 1 (MelanA or MART1), Mesothelin (MSLN), MUC1, MUC16, MHC class I chain related proteins A (MICA), MHC class I chain related proteins B (MICB), neural cell adhesion molecule (NCAM), cancer/testis antigen 1 (NY-ESO-1), polysialic acid; placenta-specific 1 (PLAC1), preferentially expressed antigen in melanoma (PRAME), prostate stem cell antigen (PSCA), prostate-specific membrane antigen (PSMA), receptor tyrosine kinase-like orphan receptor 1 (ROR1), synovial sarcoma, X breakpoint 2 (SSX2), Survivin, tumor associated glycoprotein 72 (TAG72), tumor endothelial marker 1 (TEM1/CD248), tumor endothelial marker 7-related (TEM7R), trophoblast glycoprotein (TPBG), UL16-binding protein (ULBP) 1, ULBP2, ULBP3, ULBP4, ULBP5, ULBP6, vascular endothelial growth factor receptor 2 (VEGFR2), or Wilms tumor 1 (WT-1).

### VII. Therapeutic Methods

The modified immune effector cells (e.g., T cells) manufactured by the methods provided herein provide improved adoptive immunotherapy for use in the treatment of various conditions including, without limitation, cancer, infectious disease, autoimmune disease, inflammatory disease, and immunodeficiency. In particular embodiments, the specificity of a primary T cell is redirected to tumor or cancer cells by genetically modifying the primary T cell with an engineered antigen receptor (e.g., TCR, DARIC, or CAR) contemplated herein. In one embodiment, the present disclosure includes a type of cellular therapy where immune effector cells (e.g., T cells) are modified to express an engineered antigen receptor (e.g., TCR, DARIC, or CAR) that targets cancer cells that express a target antigen, and the modified immune effector cells (e.g., T cells) are infused to a recipient in need thereof. The infused cells can kill tumor cells in the recipient. Unlike antibody therapies, engineered antigen receptor (e.g., TCR, DARIC, or CAR) modified immune effector cells (e.g., T cells) are able to replicate in vivo; thus, contributing to long-term persistence that can lead to sustained cancer therapy.

In one embodiment, the engineered antigen receptor (e.g., TCR, DARIC, or CAR) immune effector cells (e.g., T cells) of the disclosure can undergo robust in vivo immune effector cell (e.g., T cell) expansion and can persist for an extended amount of time. In another embodiment, the engineered antigen receptor (e.g., TCR, DARIC, or CAR) immune effector cells (e.g., T cells) of the disclosure evolve into specific memory T cells that can be reactivated to inhibit any additional tumor formation or growth.

In various certain embodiments, methods of treating a cancer, infectious disease, autoimmune disease, inflammatory disease, or immunodeficiency in a subject in need thereof, which include administering to the subject a therapeutically effect amount of an immune effector cell composition provided herein, are provided. In particular embodiments, the cancer is selected from the group consisting of Wilms' tumor, Ewing sarcoma, a neuroendocrine tumor, a glioblastoma, a neuroblastoma, a melanoma, skin cancer, breast cancer, colon cancer, rectal cancer, prostate cancer, liver cancer, renal cancer, pancreatic cancer, lung cancer, biliary cancer, cervical cancer, endometrial cancer, esophageal cancer, gastric cancer, head and neck cancer, medullary thyroid carcinoma, ovarian cancer, glioma, lymphoma, leukemia, myeloma, acute lymphoblastic leukemia, acute myelogenous leukemia, chronic lymphocytic leukemia, chronic myelogenous leukemia, Hodgkin's lymphoma, non-Hodgkin's lymphoma, and urinary bladder cancer.

In particular embodiments, compositions including an immune effector cell genetically modified with a vector comprising a promoter operably linked to a polynucleotide encoding a TCR, DARIC, or CAR are used in the treatment of solid tumors or cancers including, without limitation, liver cancer, pancreatic cancer, lung cancer, breast cancer, bladder cancer, brain cancer, bone cancer, thyroid cancer, kidney cancer, or skin cancer. In some embodiments, the cancer is associated with or caused by a viral infection, e.g., infection by CMV, HPV, or EBV.

In particular embodiments, compositions comprising an immune effector cell genetically modified with a vector comprising a promoter operably linked to a polynucleotide encoding an engineered antigen receptor (e.g., TCR, DARIC, or CAR) that comprises an antigen-specific binding domain that binds an epitope, for example, of PSCA, EPHA2, EGFRvIII, CSPG4, BCMA, CD33, CD123, CLL1, GD2, HER2, or MUC1. In some examples, the extracellular binding domain of the engineered antigen receptor binds an epitope of PSCA or MUC1 and the immune effector cells are used in the treatment of various cancers, including but not limited to, pancreatic, bladder, and lung. In one embodiment, the cancer is pancreatic cancer and the extracellular binding domain of the engineered antigen receptor binds an epitope of PSCA or MUC1. In some embodiments, the cancer is bladder cancer and the extracellular binding domain of the engineered antigen receptor binds an epitope of PSCA or MUC1. In further embodiments, the cancer is glioblastoma multiforme and the extracellular binding domain of the engineered antigen receptor binds an epitope of EPHA2, EGFRvIII, or CSPG4. In particular embodiments, the cancer is lung cancer and the extracellular binding domain of the engineered antigen receptor binds an epitope of PSCA or GD2. In certain embodiments, the cancer is breast cancer and the extracellular binding domain of the engineered antigen receptor binds an epitope of CSPG4 or HER2. In additional embodiments, the cancer is melanoma and the extracellular binding domain of the engineered antigen receptor binds an epitope of CSPG4 or GD2. In particular embodiments, the cancer is a hematological malignancy and the binding domain of the engineered antigen receptor binds an epitope of BCMA CD33, CD123, or CLL1. The engineered antigen receptor can be anti-BCMA02, wherein the engineered antigen receptor binds an epitope of BCMA.

In particular embodiments, compositions comprising an immune effector cell genetically modified with a vector comprising a promoter operably linked to a polynucleotide encoding an engineered antigen receptor (e.g., TCR, DARIC, or CAR) are used in the treatment of liquid tumors, including but a leukemia, including acute leukemia (e.g., ALL, AML, and myeloblastic, promyelocytic, myelomonocytic, monocytic and erythroleukemia), chronic leukemias (e.g., CLL, SLL, CML, HCL), polycythemia vera, lymphoma, Hodgkin's disease, non-Hodgkin's lymphoma, multiple myeloma, Waldenstrom's macroglobulinemia, and heavy chain disease. Thus, in some examples, a hematological malignancy is treated, such as multiple myeloma (MM), chronic lymphocytic leukemia (CLL), acute myeloid leukemia (AML), or non-Hodgkin's lymphoma (NHL). In particular embodiments, the MM is overt multiple myeloma, smoldering multiple myeloma, plasma cell leukemia, non-secretory myeloma, IgD myeloma, osteosclerotic myeloma, solitary plasmacytoma of bone, or extramedullary plasmacytoma. In certain embodiments, the NHL is: Burkitt lymphoma, chronic lymphocytic leukemia/small lymphocytic lymphoma (CLL/SLL), diffuse large B-cell lymphoma, follicular lymphoma, immunoblastic large cell lymphoma, precursor B-lymphoblastic lymphoma, or mantle cell lymphoma. In some such examples, the engineered antigen receptor includes an extracellular binding domain that binds BCMA, CD33, CD123, or CLL1.

In particular embodiments, compositions comprising an immune effector cell genetically modified with a vector comprising a promoter operably linked to a polynucleotide encoding an engineered antigen receptor (e.g., TCR, DARIC, or CAR) are used in the treatment of B-cell malignancies, including but not limited to multiple myeloma (MM), non-Hodgkin's lymphoma (NHL), and chronic lymphocytic leukemia (CLL).

Multiple myeloma is a B-cell malignancy of mature plasma cell morphology characterized by the neoplastic transformation of a single clone of these types of cells. These plasma cells proliferate in BM and may invade adjacent bone and sometimes the blood. Variant forms of multiple myeloma include overt multiple myeloma, smoldering multiple myeloma, plasma cell leukemia, non-secretory myeloma, IgD myeloma, osteosclerotic myeloma, solitary plasmacytoma of bone, and extramedullary plasmacytoma (see, for example, Braunwald, et al. (eds), Harrison's Principles of Internal Medicine, 15th Edition (McGraw-Hill 2001)).

Non-Hodgkin lymphoma encompasses a large group of cancers of lymphocytes (white blood cells). Non-Hodgkin lymphomas can occur at any age and are often marked by lymph nodes that are larger than normal, fever, and weight loss. There are many different types of non-Hodgkin lymphoma. For example, non-Hodgkin's lymphoma can be divided into aggressive (fast-growing) and indolent (slow-growing) types. Although non-Hodgkin lymphomas can be derived from B-cells and T-cells, as used herein, the term “non-Hodgkin lymphoma” and “B-cell non-Hodgkin lymphoma” are used interchangeably. B-cell non-Hodgkin lymphomas (NHL) include Burkitt lymphoma, chronic lymphocytic leukemia/small lymphocytic lymphoma (CLL/SLL), diffuse large B-cell lymphoma, follicular lymphoma, immunoblastic large cell lymphoma, precursor B-lymphoblastic lymphoma, and mantle cell lymphoma. Lymphomas that occur after bone marrow or stem cell transplantation are usually B-cell non-Hodgkin lymphomas.

Chronic lymphocytic leukemia (CLL) is an indolent (slow-growing) cancer that causes a slow increase in immature white blood cells called B lymphocytes, or B cells. Cancer cells spread through the blood and bone marrow, and can also affect the lymph nodes or other organs such as the liver and spleen. CLL eventually causes the bone marrow to fail. Sometimes, in later stages of the disease, the disease is called small lymphocytic lymphoma.

In particular embodiments, methods comprising administering a therapeutically effective amount of modified immune effector cells (e.g., T cells) contemplated herein or a composition comprising the same, to a patient in need thereof, alone or in combination with one or more therapeutic agents, are provided. In certain embodiments, the cells of the disclosure are used in the treatment of patients at risk for developing a cancer. Thus, the present disclosure provides methods for the treatment or prevention of a cancer comprising administering to a subject in need thereof, a therapeutically effective amount of the modified immune effector cells (e.g., T cells) of the disclosure.

In one embodiment, a method of treating a cancer in a subject in need thereof includes administering an effective amount, e.g., therapeutically effective amount of a composition comprising genetically modified immune effector cells contemplated herein. The quantity and frequency of administration will be determined by such factors as the condition of the patient, and the type and severity of the patient's disease, although appropriate dosages may be determined by clinical trials.

In one embodiment, the amount of immune effector cells (e.g., T cells) in the composition (generated using this disclosed methods) administered to a subject is at least 0.1×10⁵ cells, at least 0.5×10⁵ cells, at least 1×10⁵ cells, at least 5×10⁵ cells, at least 1×10⁶ cells, at least 0.5×10⁷ cells, at least 1×10⁷ cells, at least 0.5×10⁸ cells, at least 1×10⁸ cells, at least 0.5×10⁹ cells, at least 1×10⁹ cells, at least 2×10⁹ cells, at least 3×10⁹ cells, at least 4×10⁹ cells, at least 5×10⁹ cells, or at least 1×10¹⁰ cells. In particular embodiments, about 1×10⁷ CAR, TRC, or DARIC immune effector cells (e.g., T cells) to about 1×10⁹ CAR, TRC, or DARIC immune effector cells (e.g., T cells), about 2×10⁷ CAR, TRC, or DARIC immune effector cells (e.g., T cells) to about 0.9×10⁹ CAR, TRC, or DARIC immune effector cells (e.g., T cells), about 3×10⁷ CAR, TRC, or DARIC immune effector cells (e.g., T cells) to about 0.8×10⁹ CAR, TRC, or DARIC immune effector cells (e.g., T cells), about 4×10⁷ CAR, TRC, or DARIC immune effector cells (e.g., T cells) to about 0.7×10⁹ CAR, TRC, or DARIC immune effector cells (e.g., T cells), about 5×10⁷ CAR, TRC, or DARIC immune effector cells (e.g., T cells) to about 0.6×10⁹ CAR, TRC, or DARIC immune effector cells (e.g., T cells), or about 5×10⁷ CAR, TRC, or DARIC immune effector cells (e.g., T cells) to about 0.5×10⁹ CAR, TRC, or DARIC immune effector cells (e.g., T cells) are administered to a subject.

In one embodiment, the amount of T cells in the composition (generated using this disclosed methods) administered to a subject is at least 0.1×10⁴ cells/kg of bodyweight, at least 0.5×10⁴ cells/kg of bodyweight, at least 1×10⁴ cells/kg of bodyweight, at least 5×10⁴ cells/kg of bodyweight, at least 1×10⁵ cells/kg of bodyweight, at least 0.5×10⁶ cells/kg of bodyweight, at least 1×10⁶ cells/kg of bodyweight, at least 0.5×10⁷ cells/kg of bodyweight, at least 1×10⁷ cells/kg of bodyweight, at least 0.5×10⁸ cells/kg of bodyweight, at least 1×10⁸ cells/kg of bodyweight, at least 2×10⁸ cells/kg of bodyweight, at least 3×10⁸ cells/kg of bodyweight, at least 4×10⁸ cells/kg of bodyweight, at least 5×10⁸ cells/kg of bodyweight, or at least 1×10⁹ cells/kg of bodyweight. In particular embodiments, about 1×10⁶ CAR, TRC, or DARIC immune effector cells (e.g., T cells)/kg of bodyweight to about 1×10⁸ CAR, TRC, or DARIC immune effector cells (e.g., T cells)/kg of bodyweight, about 2×10⁶ CAR, TRC, or DARIC immune effector cells (e.g., T cells)/kg of bodyweight to about 0.9×10⁸ CAR, TRC, or DARIC immune effector cells (e.g., T cells)/kg of bodyweight, about 3×10⁶ CAR, TRC, or DARIC immune effector cells (e.g., T cells)/kg of bodyweight to about 0.8×10⁸ CAR, TRC, or DARIC immune effector cells (e.g., T cells)/kg of bodyweight, about 4×10⁶ CAR, TRC, or DARIC immune effector cells (e.g., T cells)/kg of bodyweight to about 0.7×10⁸ CAR, TRC, or DARIC immune effector cells (e.g., T cells)/kg of bodyweight, about 5×10⁶ CAR, TRC, or DARIC immune effector cells (e.g., T cells)/kg of bodyweight to about 0.6×10⁸ CAR, TRC, or DARIC immune effector cells (e.g., T cells)/kg of bodyweight, or about 5×10⁶ CAR, TRC, or DARIC immune effector cells (e.g., T cells)/kg of bodyweight to about 0.5×10⁸ CAR, TRC, or DARIC immune effector cells (e.g., T cells)/kg of bodyweight are administered to a subject.

One of ordinary skill in the art would recognize that multiple administrations of the compositions of the disclosure may be required to effect the desired therapy. For example a composition may be administered 1, 2, 3, 4, 5, 6, 7, 8, 9, or 10 or more times over a span of 1 week, 2 weeks, 3 weeks, 1 month, 2 months, 3 months, 4 months, 5 months, 6 months, 1 year, 2 years, 5, years, 10 years, or more.

In certain embodiments, it may be desirable to administer activated immune effector cells (e.g., T cells) to a subject and then subsequently redraw blood (or have an apheresis performed), activate T cells therefrom according to the present disclosure, and reinfuse the patient with these activated and expanded immune effector cells (e.g., T cells). This process can be carried out multiple times every few weeks. In certain embodiments, immune effector cells (e.g., T cells) can be activated from blood draws of from 10 cc to 400 cc. In certain embodiments, T cells are activated from blood draws of 20 cc, 30 cc, 40 cc, 50 cc, 60 cc, 70 cc, 80 cc, 90 cc, 100 cc, 150 cc, 200 cc, 250 cc, 300 cc, 350 cc, or 400 cc or more. Not to be bound by theory, using this multiple blood draw/multiple reinfusion protocol may serve to select out certain populations of immune effector cells (e.g., T cells).

The administration of the compositions contemplated herein may be carried out in any convenient manner, including by aerosol inhalation, injection, ingestion, transfusion, implantation or transplantation. In a preferred embodiment, compositions are administered parenterally. The phrases “parenteral administration” and “administered parenterally” as used herein refers to modes of administration other than enteral and topical administration, usually by injection, and includes, without limitation, intravascular, intravenous, intramuscular, intraarterial, intrathecal, intracapsular, intraorbital, intratumoral, intracardiac, intradermal, intraperitoneal, transtracheal, subcutaneous, subcuticular, intraarticular, subcapsular, subarachnoid, intraspinal and intrasternal injection and infusion. In one embodiment, the compositions contemplated herein are administered to a subject by direct injection into a tumor, lymph node, or site of infection.

In one embodiment, a subject in need thereof is administered an effective amount of a composition to increase a cellular immune response to a cancer in the subject. The immune response may include cellular immune responses mediated by cytotoxic T cells capable of killing infected cells, regulatory T cells, and helper T cell responses. Humoral immune responses, mediated primarily by helper T cells capable of activating B cells thus leading to antibody production, may also be induced. A variety of techniques may be used for analyzing the type of immune responses induced by the compositions of the present disclosure, for example see Current Protocols in Immunology, Edited by: John E. Coligan, Ada M. Kruisbeek, David H. Margulies, Ethan M. Shevach, Warren Strober (2001) John Wiley & Sons, NY, N.Y.

In the case of T cell-mediated killing, CAR-ligand binding initiates CAR signaling to the T cell, resulting in activation of a variety of T cell signaling pathways that induce the T cell to produce or release proteins capable of inducing target cell apoptosis by various mechanisms. These T cell-mediated mechanisms include (but are not limited to) the transfer of intracellular cytotoxic granules from the T cell into the target cell, T cell secretion of pro-inflammatory cytokines that can induce target cell killing directly (or indirectly via recruitment of other killer effector cells), and up regulation of death receptor ligands (e.g. FasL) on the T cell surface that induce target cell apoptosis following binding to their cognate death receptor (e.g. Fas) on the target cell.

In one embodiment, the disclosure provides a method of treating a subject diagnosed with a cancer, comprising removing immune effector cells from the subject, genetically modifying said immune effector cells with a vector comprising a nucleic acid encoding an engineered TCR, DARIC, or CAR as contemplated herein, thereby producing a population of modified immune effector cells, and administering the population of modified immune effector cells to the same subject. In a one embodiment, the immune effector cells comprise T cells.

In certain embodiments, the present disclosure also provides methods for stimulating an immune effector cell mediated immune modulator response to a target cell population in a subject comprising the steps of administering to the subject an immune effector cell population expressing a nucleic acid construct encoding an engineered TCR, DARIC, or CAR molecule.

The methods for administering the cell compositions described herein includes any method which is effective to result in reintroduction of ex vivo genetically modified immune effector cells that either directly express an engineered TCR, DARIC, or CAR in the subject or on reintroduction of the genetically modified progenitors of immune effector cells that on introduction into a subject differentiate into mature immune effector cells that express the engineered TCR, DARIC, or CAR. One method comprises transducing peripheral blood immune effector cells (e.g., T cells) ex vivo with a nucleic acid construct in accordance with the disclosure and returning the transduced cells into the subject.

In some examples, the methods provided herein include administration of one or more additional therapeutic agents, such as one or more anti-cancer agents provided herein. In some examples, the one or more additional therapeutic agents is a therapeutically effective amount of one or more T cell agonists, such as an agonist of 4-1BB (CD137), OX40, and/or GITR. In one example, an OX40 agonist is an anti-OX40 antibody, such as a monoclonal antibody (mAb) (e.g., PF-04518600, MEDI-6469, MEDI-0562, MEDI-6383, MOXR-0916, BMS 986178, or GSK3174998). Mimicking the natural OX40 ligand (OX40L), anti-OX40 monoclonal antibody selectively binds to and activates the OX40 receptor. In one example, an OX40 agonist is an OX40 ligand, OX40L, such as a natural ligand (such as a human OX40L). In one example, an OX40 agonist is an OX40 aptamer. In one example, a 4-1BB agonist is a 4-1BB agonist antibody, such as a mAb. Specific agonist mAbs that can be used with the disclosed methods include PF-05082566 (utomilumab), and BMS-663513 (Urelumab). In one example, a 4-1BB agonist is a 4-1BB ligand (4-1BBL), such as a natural 4-1BBL (such as the human 4-1IBBL) or a streptavidinated 4-1BBL (SA-4-1BBL) complex. In one example, a 4-1BB agonist is a 4-1BB aptamer. GITR (glucocorticoid-induced tumor necrosis factor (TNF) receptor, or TNFRSF18) is a type I transmembrane protein with homology to other TNF receptor family members such as OX40, CD27, and 4-1BB. In one example, a GITR agonist is a GITR agonist antibody, such as a mAb. Specific GITR agonist mAbs that can be used with the disclosed methods include DTA-1, TRX518, MK-4166, MK-1248, AMG 228, INCAGN01876, GWN323 (from Novartis), CK-302 (Checkpoint Therapeutics) and BMS-986156. In one example, a GITR agonist is a GITR ligand (GITRL), such as a natural GITRL or a multivalent GITR ligand fusion protein. In one example, the GITR agonist is MEDI1873, a hexameric GITRL molecule with a human IgG1 Fc domain. In one example, GITR agonist is a GITR aptamer.

The disclosed methods can treat a tumor in the subject by reducing the volume or weight of the tumor, reducing the number of metastases, reducing the size or weight of a metastasis, or combinations thereof. In some examples a metastasis is cutaneous or subcutaneous. Thus, in some examples, administration of the disclosed immune effector cells (e.g., T cells) (alone or in combination with another anti-cancer therapy) treats a tumor in a subject by reducing the size or volume of the tumor by at least 10%, at least 20%, at least 30%, at least 40%, at least 50%, at least 60%, at least 70%, at least 75%, at least 80%, at least 90%, at least 95%, at least 98% or at least 99%, for example as compared to no administration of the disclosed immune effector cells (e.g., T cells) or administration of immune effector cells (e.g., T cells) not generated using the disclosed methods (e.g., immune effector cells cultured in the absence of the one or more of the metabolic enhancers). In some examples, administration of disclosed immune effector cells (e.g., T cells) treats a tumor in a subject by reducing the weight of the tumor by at least 10%, at least 20%, at least 30%, at least 40%, at least 50%, at least 60%, at least 70%, at least 75%, at least 80%, at least 90%, at least 95%, at least 98% or at least 99%, for example as compared to no administration of the disclosed immune effector cells (e.g., T cells) or administration of immune effector cells (e.g., T cells) not generated using the disclosed methods (e.g., immune effector cells cultured in the absence of the one or more of the metabolic enhancers). In some examples, administration of the disclosed immune effector cells (e.g., T cells) treats a tumor in a subject by reducing the size or volume of a metastasis by at least 10%, at least 20%, at least 30%, at least 40%, at least 50%, at least 60%, at least 70%, at least 75%, at least 80%, at least 90%, at least 95%, at least 98% or at least 99% for example as compared to no administration of the disclosed immune effector cells (e.g., T cells) or administration of immune effector cells (e.g., T cells) not generated using the disclosed methods (e.g., immune effector cells cultured in the absence of the one or more of the metabolic enhancers). In some examples, administration of the disclosed immune effector cells (e.g., T cells) treats a tumor in a subject by reducing the number of metastases by at least 10%, at least 20%, at least 30%, at least 40%, at least 50%, at least 60%, at least 70%, at least 75%, at least 80%, at least 90%, at least 95%, at least 98% or at least 99%, for example as compared to no administration of the disclosed immune effector cells (e.g., T cells) or administration of immune effector cells (e.g., T cells) not generated using the disclosed methods (e.g., immune effector cells cultured in the absence of the one or more of the metabolic enhancers). In some examples, administration of the disclosed immune effector cells (e.g., T cells) increases the survival time of a subject s by at least 10%, at least 20%, at least 30%, at least 40%, at least 50%, at least 60%, at least 70%, at least 75%, at least 80%, at least 90%, at least 95%, at least 98%, at least 99%, at least 150%, at least 200%, or at least 500%, for example as compared to no administration of the disclosed immune effector cells (e.g., T cells) or administration of immune effector cells (e.g., T cells) not generated using the disclosed methods (e.g., immune effector cells cultured in the absence of the one or more of the metabolic enhancers). In some examples, combinations of these effects are achieved.

In some examples, the methods include monitoring tumor growth in response to treatment.

### EXAMPLES

The following examples are provided to illustrate certain particular features and/or embodiments. These examples should not be construed to limit the disclosure to the particular features or embodiments described. Those of skill in the art will readily recognize a variety of noncritical parameters that could be changed or modified to yield essentially similar results. One skilled in the art will appreciate that other immune effector cells can be used in place of the T cells described below.

### Example 1

### Materials and Methods

This example provides materials and methods used in the examples below.

### A. Apparatus and Methods for Measuring Respiratory Capacity in a Microplate Containing a Cell Culture

The methods provided herein can include measurement of plural extracellular solutes and adjacent living cells disposed, for example, in a well of a multiwell microplate. In one example, this data can be obtained using materials from Seahorse BioScience, Inc., of Billerica, Mass., under the trademark CellDoctor™.

This apparatus can include an array of submersible sensors that enable rapid coordinated sensor stabilization within multiple cell-containing wells. It also includes compound storage and delivery apparatus, a pneumatic multiplexer, a structure for adding fluids to subsets or all of the multiple wells simultaneously, and a sensor structure permitting non-destructive measurement of the effect of addition of exogenous fluid to the respective wells, in combination with the ability to make and repeat measurements rapidly. Furthermore, the apparatus is designed to exploit a cartridge structure which permits repeated use of the apparatus for disparate cellular assays without requiring intermediate cleaning, and while eliminating the possibility of cross contamination between tests.

In use, the first step in a measurement sequence with the Seahorse XF instrument is the exchange of cell media to a type containing neatly no bicarbonate or other pH buffer in order to maximize the pH change caused by cellular proton flux.

Next, measurement probes fitted with an optical oxygen and pH sensor were placed in each well, and were raised and lowered to mix the cell media for approximately five minutes. Third, each probe was lowered into the cell media to stop at a precise point above the cells such that approximately 25 μL of media was sequestered in a way that impeded the diffusion of oxygen molecules and protons to the larger volume of media in the well. Fourth, a series of optical measurements of the two sensors on the probe bottom (in contact with the small volume of media above the cells) were made at intervals of 8 seconds and for a period of five minutes. Typically, the dissolved oxygen level decreased by approximately 10% and the pH decreased by approximately 0.1 units. A simple linear fit to the rate of change of oxygen and pH were used to determine the OCR and ECAR for each well.

Next, the probes were elevated and then reciprocated to re-mix the depleted media with the larger residual volume in the well. Typically, both the dissolved oxygen level and pH of the mixed media returned to the starting values. This basal rate measurement was typically repeated. T cells were typically seeded in 96-well tissue culture microplates at a density of 10,000 cells per well.

In cases where PDHK1 inhibitors were to be used, for example, 100 μL of a 10× compound solution was added to the cell media while the sensor probe was elevated but resident in the cell media. A mix cycle and measurement cycle were then performed and both were repeated again. In some cases, second and third drug compounds were added with mix and measure cycles for each. Baseline metabolic rates were reported in pmol/min for OCR and mpH/min for ECAR. At the completion of each assay, cells were suspended from the well bottoms using Trypsin, and a viable cell number count was obtained using an automated Trypan Blue dye exclusion type cell counter.

### B. Tumor Models

Mice were injected with B16.F10 melanoma (1.25×10⁵ cells) or A549 (1.25×10⁵ cells). Tumors were measured regularly with digital calipers and tumor volume calculated. Tumors and lymph nodes were harvested for analysis. TILs were prepared using a Percoll® density gradient from tumor samples after mechanical disruption. Therapeutic B116 experiments were conducted by injecting 1.25×10⁵ B16 melanoma cells i.d. and waiting until tumors were palpable (5 days).

### C. Signaling Analysis

For flow cytometry, cells were stimulated with anti-CD3e/anti-CD28 coated beads and either purified with conventional T cells or Sema4a-Ig beads for various times, then fixed with 1% PFA for 15 minutes at 37 °C. Cells were then permeabilized in ice-cold 90% MeOH for 20 min at −20 °C. After extensive washing in PBS, cells were blocked with 10% normal mouse serum in PBS for 10 minutes at RT. Cells were then stained with antibodies in 1% BSA, in PBS (pAkt (T308), pAkt (S473)) for 1 hour at RIT in the dark. Finally, cells were stained with appropriate secondary antibodies for 30 minutes at R.T in the dark, then washed and analyzed. For immunoblot analysis, cells were expanded with 1 ng/mL phorbol-13-myristol acetate and 10 ng/mL ionomycin with 500 U rhIL-2 for 3 days, then washed extensively with media, and expanded to 10× volume in 500 U rhIL-2. After an overnight rest with no IL-2, cells were stimulated with plate-bound anti-CD3, soluble anti-CD28 and bead-bound Sema4a-Ig for 3 hours, then lysed in whole cell lysis buffer (1% NP40, 5 mM EDTA, 5 mM EGTA, TWEEN®-20) for 15 min on ice. In some experiments, 3×10⁶ DCA treated pmel T cells were lysed in a larger volume, and cleared lysates were incubated with Protein G beads for 3 hours to “preclear” the lysate. IFN-γ+ was immunoprecipitated using a polyclonal anti-IFN-γ+ antibody overnight followed by a 3 hour incubation with Protein G beads. Beads were washed with lysis buffer before elution and reduction prior to immunoblotting. Briefly, precipitates or input lysates were incubated at 100 °C with 2-mercaptoethanol and 4×LDS sample buffer (Life Technologies), then loaded into 4-12% Bis-Tris NuPAGE® gels (Life Technologies), and run for 1 hour at 200V Separated gels were electrotransferred to PVDF membranes using the Criterion® Gel Blotting System (Biorad), and blocked for 1 hour at room temperature with 3% BSA in TBS supplemented with 0.1% TWEEN®-20. In an alternate approach, flow cytometry measurements were analyzed using the MitoTracker® Deep Red FM device as described in Example 8 below.

### D. Human Cells

Human peripheral blood was obtained from leukapharesis rings isolated from healthy donors by Vitalant (Pittsburgh, PA).

Patient samples and PBMC samples were obtained from melanoma patients who were enrolled on institutional review board approved protocols Cells from 5 different patients were used. PBMC were cryopreserved in 90% FBS plus 10% DMSO (Sigma, St. Louis, MO).

The following CD19-expressing immortalized cell lines were used: A549 (lung carcinoma). All cell lines were maintained in R10 medium.

E. Standard Mouse T Cell Expansion with DCA

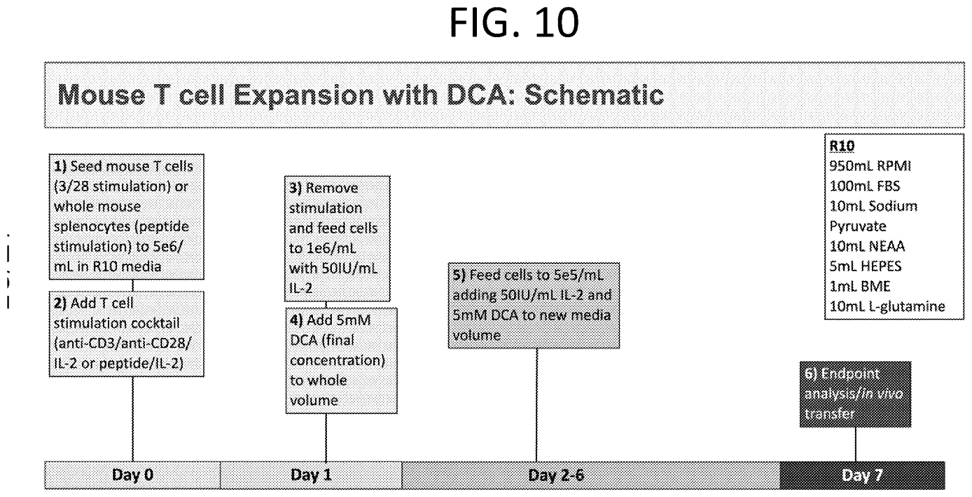

Peripheral blood mononuclear (PBMCs) were harvested from normal donors and labeled with a fluorescent dye (CellTrace® Violet, Molecular Probes), whose intensity was progressively diluted with each cell division. The labeled PBMCs were used as a source of cells for T cell expansion. As shown in FIG. 10, mouse T cells were then expanded with DCA as follows. On the first day (“day 0”), 1) mouse T cells were seeded with CD3 and CD28 antibodies (Miltenyi Biotec) or whole mouse splenocytes (peptide stimulation) to a concentration of 5×10⁶ cells/mL in R10 media, and 2) T cell stimulation cocktail (anti-CD3, anti-CD28, IL-2 and/or peptide IL-2) was added. After 24 hours (“day 1”), 3) stimulation was removed and cells were fed to a concentration of 1×10⁶ cells/mL with 50 IU/mL IL-2 (CellGenix), and 4) DCA was added to a final concentration of 5 mM DCA. On days 2-6, 5) cells were fed to a concentration of 5×10⁵ cells/mL adding 50 IU/mL IL-2 and 5 mM DCA to new media. On day 7, 6) endpoint analysis and in vivo transfer were carried out. Standard R10 media culture includes: 950 mL RPMI, 100 mL FBS, 10 mL Sodium Pyruvate, 10 mL NEAA, 5 mL HEPES, 1 mL BME, 10 mL and L-glutamine.

F. Standard Human CAR-T Cell Production with DCA

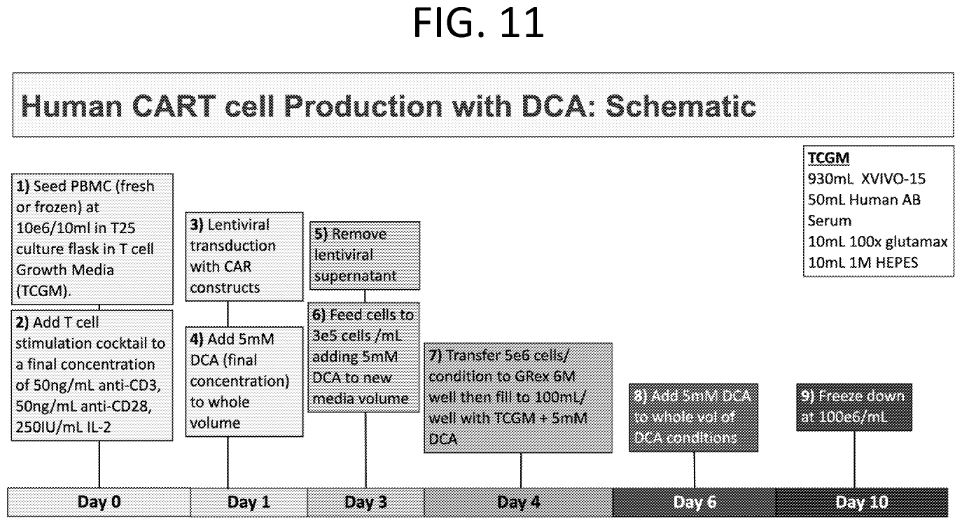

As shown in FIG. 11, human CAR-T cells can be expanded with DCA as follows. On the first day (“day 0”), 1) fresh or frozen peripheral blood mononuclear (PBMCs) are seeded at 10×10⁶ cells/10 mL in a T25 culture flask in T Cell Growth Media (TCGM), and 2) T cell stimulation cocktail is added to a final concentration of 50 ng/mL anti-CD3, 50 ng/mL anti-CD28, and 250 IU/mL IL-2. After 24 hours (“day 1”), 3) lentiviral transduction is carried out with CAR constructs, and 4) DCA is added to a final concentration of 5 mM DCA. On day 3, 5) lentiviral supernatant is removed, and 6) cells are fed with new media to a concentration of 3×10⁵ cells/mL and 5 mM DCA. On day 4, 7) a transfer of 5×10⁶ cells/condition is made to G-Rex® 6 multi-well plates, filling TCGM+5 mM DCA to a volume of 100 mL/well. On day 6, 8) DCA is added to a final concentration of 5 mM DCA. On day 10, 9) cells are frozen down to a concentration of 100×10⁶ cells/mL. Standard T Cell Growth Media includes: 930 mL X-VIVO®-15 cell medium, 50 mL Human AB Serum, 10 mL 100× GlutaMAX® supplement, and 10 mL 1M HEPES.

### Example 2

### T Cells Expanded in DCA Show Improved Survival

This experiment determined the effect of PDHK1 inhibition on T cell survival.

The impact of a PDHK1 inhibitor (DCA) on T cell survival was assessed by measuring expression of anti-apoptotic BLC2. Peripheral blood mononuclear (PBMCs) were harvested from normal donors and labeled with a fluorescent dye (CellTrace® Violet, Molecular Probes), whose intensity is progressively diluted with each cell division. The labeled PBMCs were used as a source of cells for T cell expansion. The T cells were activated and expanded as described in Example 1, except the labeled PBMCs were cultured in media containing IL-2 for 48 hours, and the T cells were cultured in DCA or control conditions for 10 days. Experimental expansion conditions included culturing in 5 mM DCA or 10 mM DCA. Control conditions included 5 mM of glycolysis inhibitor 2-deoxyglucose (2DG), low glucose media, or PBS. The impact of DCA on T cell survival was assayed by evaluating CellTrace® Violet dilution with flow cytometry. Fresh X-VIVO®-15-based culture medium containing IL-2 and DCA or 2DG was added to the T cell cultures every two to three days for a total of ten days to permit outgrowth and expansion of the T cells.

Surprisingly, DCA significantly increased T cell survival after ten days of expansion. Enhanced T cell survival correlated with enhanced expression of the anti-apoptotic factor BCL2 (FIG. 3). A pairwise T test was performed on all day ten cultures (each concentration of DCA was compared to 0 mM DCA: *p<0.05, **p<0.01).

### Example 3

### DCA Promotes a Superior Metabolic State in T Cells

Metabolic flux rate experiments were performed to interrogate mitochondrial respiratory capacity and mitochondrial health in T cells expanded in PDHK1 inhibitor (DCA).

Metabolic flux rate measurements were performed using the fluid distribution system CellDoctor™ as described in Example 1. The CellDoctor™ device was configured to measure oxygen consumption rate (OCR) and extracellular acidification rate (ECAR), corresponding to proton flux, of cells that are adherent to the bottom of 24 well microplates. T cells in the experimental group (cultured in 5 mM DCA) were activated and expanded as described above.

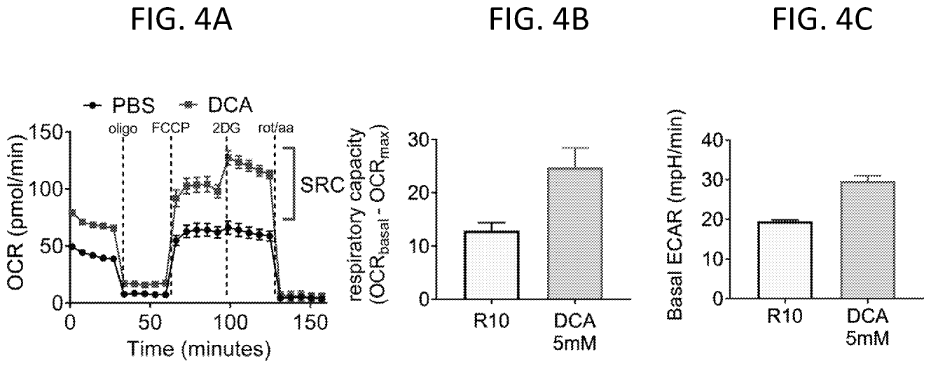

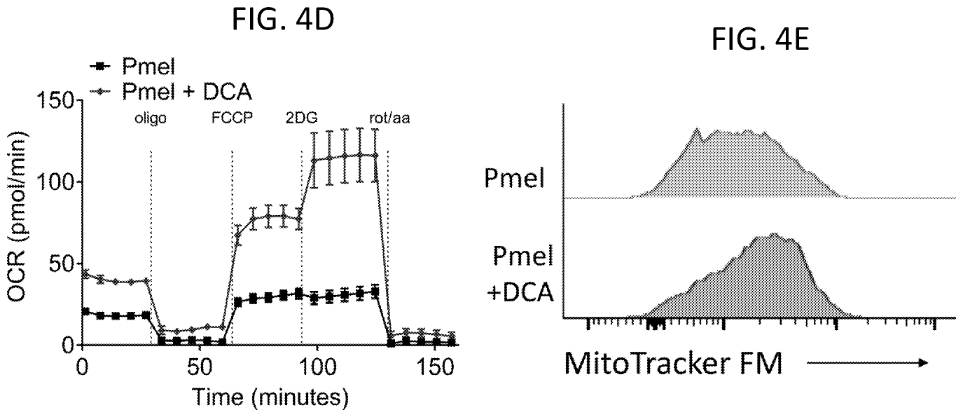

As depicted in FIG. 4A, FCCP, and 2-deoxyglucose (2DG) and were injected sequentially into wells containing T cells expanded in the presence or absence of 5 mM DCA. FIG. 4A shows that, as metabolic stress in culture was limited via the sequential injection of FCCP and 2DG, an improved metabolic phenotype was observed (e.g., OCR increased) in the DCA-treated cells versus the control cells. Similar results are shown in FIG. 4D.

FIGS. 4B-4C show a substantial increase in OCR and ECAR in T cells expanded in 5 mM DCA. Strikingly, an increase of at least 30% in OCR over baseline was observed in T cells expanded in 5 mM DCA (FIG. 4B). As shown in FIG. 4C, an increase of at least 10 mpH/min in ECAR was also observed over baseline for T cells in expanded in 5 mM DCA As shown in FIG. 4E, consistent with the observed increased respiratory capacity, mitochondrial mass (as measured by MitoTracker™) increased after culture in 5 mM DCA.

### Example 4

### Modeling an Adoptive T Cell Therapy with Melanoma Antigen

To interrogate the impact of expanding T cells in DCA on tumor growth and survival rates, an adoptive T cell therapy with melanoma antigen was developed.

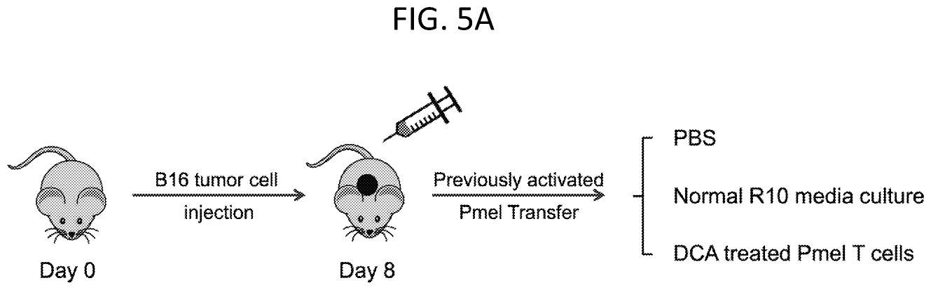

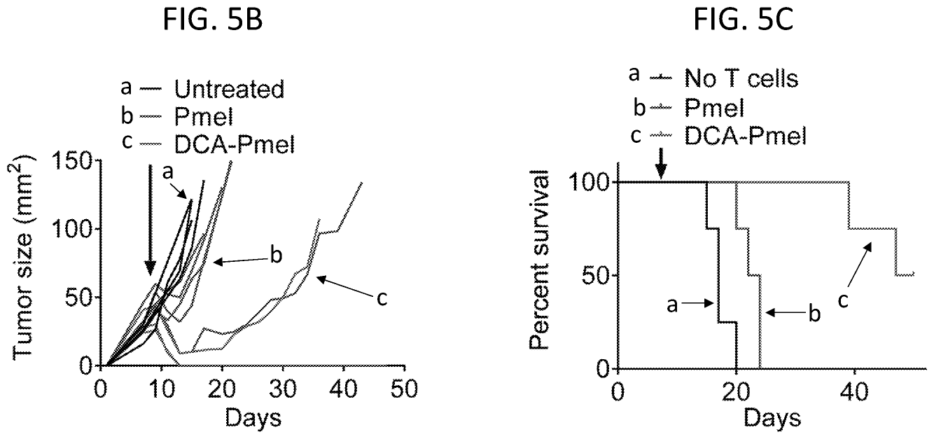

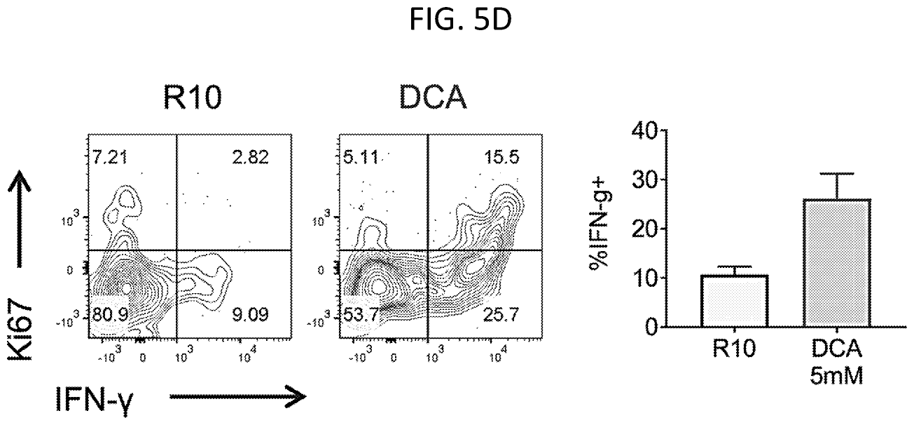

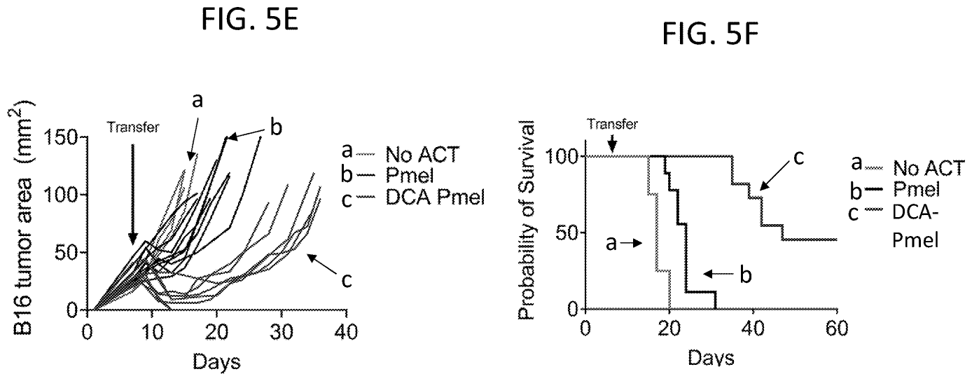

Immunodeficient mice were inoculated with human B16 tumor cells and adoptive T cell transfers were carried out after 8 days. Adoptive transfers included DCA treated pmel T cells, RO media culture (standard T cell expansion), and PBS. The Pmel T cells treated with DCA (5 mM DCA) were activated and expanded as described above. Tumor growth and survival in mice and their littermate controls was then assessed (FIGS. 5A-5B) as described above. Tumors were measured with calipers and the percent survival of mice were measured at intervals extending out to 50 days.

As shown in FIGS. 5B-5C, pmel T cells expanded in DCA (5 mM DCA) resulted in a superior metabolic phenotype. Significantly delayed tumor growth and enhanced survival was observed in mice with DCA treated Pmel T cells. Strikingly, at least 50% of the mice survived long term and produced memory to the tumor (see also, FIGS. 5E and 5F). FIG. 5D demonstrates that this observation is associated with a superior effector phenotype in the T cells that infiltrate the tumor. Specifically, T cells that infiltrate the tumor experience increased proliferation and reduced apoptosis, as assessed by Ki67 expression. In addition, T cells that infiltrate the tumor experience increased cytokine production, as assessed by intra-tumoral IFN-gamma proliferation. Signaling analysis were performed as described above.

### Example 5

### DCA Promotes Metabolic Reprogramming in Human T Cells

Metabolic flux rate experiments were performed to determine whether human T cells expanded in DCA show improved mitochondrial respiratory capacity. This example identically replicates the methods described in Example 3, except human T cells were used (derived from human healthy donor blood).

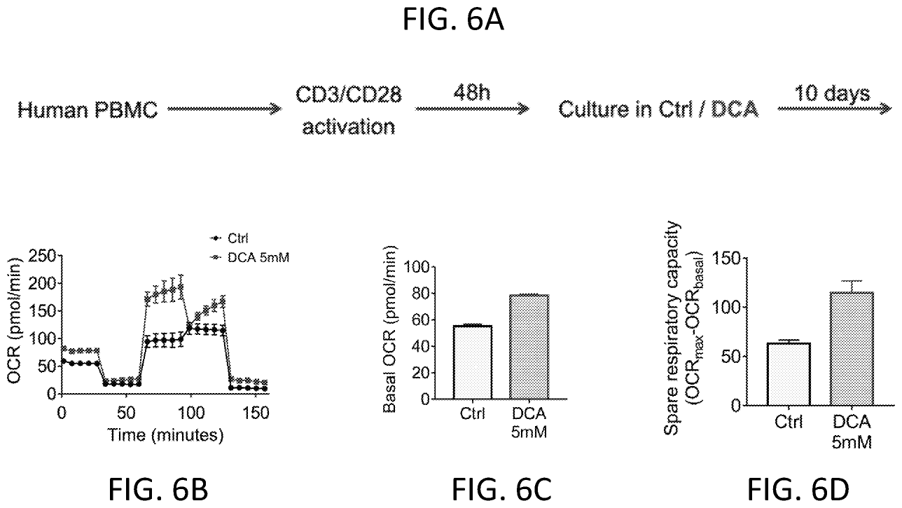

Metabolic flux rate measurements were performed using the CellDoctor™ device and methods described above. The CellDoctor™ device was configured to measure oxygen consumption rate (OCR) and extracellular acidification rate (ECAR), corresponding to proton flux, of cells that are adherent to the bottom of 24 well microplates. As described above, flux rate experiments were performed in a human T cell line expanded in either 5 mM DC-A or a control group without DCA. FIG. 6A illustrates the use of an activation step (CD3/CD28) for 48 hours and a culturing step (Ctrl/DCA) for 10 days as described in Example 2.

As depicted in FIG. 6B, a dramatic increase in OCR occurred in T cells expanded in 5 mM DCA versus control. As shown in FIG. 6C, a substantial increase of at least 20-pmol/min in OCR over baseline is observed in T cells expanded in 5 mM DCA. Strikingly, FIG. 6D shows a Spare Respiratory Capacity (“SRC”) of at least 60 (OCRmax−OCRbasal) for T cells expanded in 5 mM DCA versus control.

### Example 6

### DCA Metabolically Rescues Tumor-Infiltrating T Cells of Cancer Patients when Expanded In Vitro

Metabolic flux rate experiments were performed in T cells expanded in TILs from cancer patients after expansion in PDHK1 inhibitory conditions.

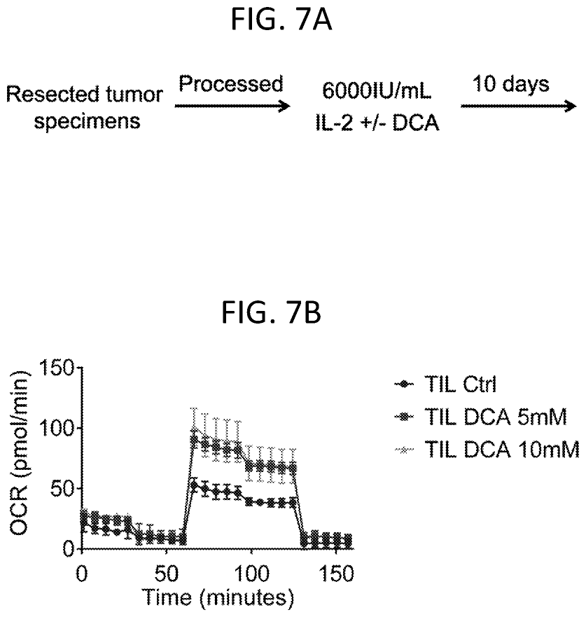

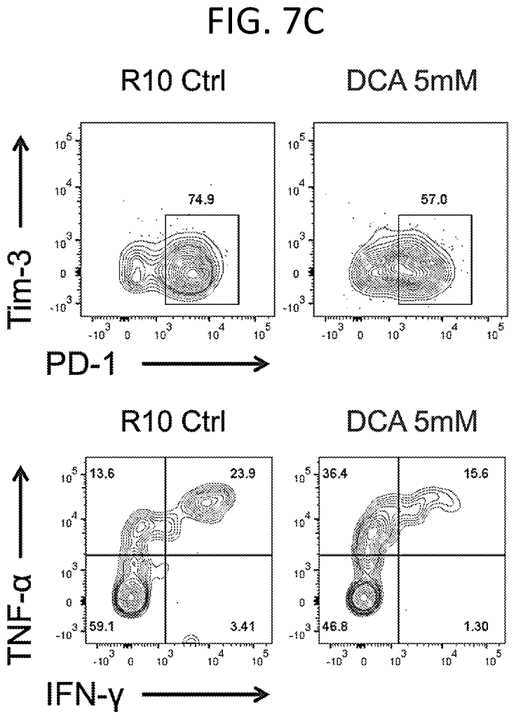

This method included obtaining a sample of tumor from a patient suffering from metastatic melanoma, making a single cell suspension, and expanding the T cells in a cell culture medium with IL-2 present in a concentration of 6000 ILU/mL+/−DCA in addition to anti-CD3 anti-CD28 antibodies for a period of 10 days. FIG. 7A shows a schematic of this in vivo experimental protocol.

Performing metabolic flux rate experiments as described above, DCA was shown to rescue tumor-infiltrating T cells. As depicted in FIG. 7B, OCR was evaluated at serial time points and it was observed that DCA-treated T cells show a superior metabolic phenotype at 5 mM DCA and 10 mM DCA relative to TIL control.

FIG. 7C also shows DCA-treated cells to have a superior metabolic phenotype as evidenced by an enhanced immune response and decreased apoptosis signaling. Decreased apoptosis signaling is reflected by an increasing ratio of: PD-1 negative T cells to PD-1 positive T cells and, Tim-3 negative T cells to Tim-3 positive T cells. Further, an enhanced immune response is reflected by an increase in cytokine production, as assessed by enhanced intra-tumoral IFN-gamma proliferation and TNF-alpha proliferation. Signaling analysis were performed as described above.

Notably, the TIL control was prepared as described above. Metabolic flux rate experiments measuring OCR were also performed as described above.

### Example 7

### DCA Metabolically Enhances HCD19 Car-T Cells and Provides Superior Efficacy in an Engineered HCD19-Expressing Solid Tumor

This example demonstrates that human anti-CD19 CAR-T cells expanded in the presence of PDHK1 inhibitor (DCA) shows improved efficacy in a solid tumor mouse model.

FMC63 (human CD19 targeted CAR) human T cells were generated in the presence or absence of DCA. These cells were used to treat mice bearing A549 lung cancer tumors that were engineered to express hCD19.

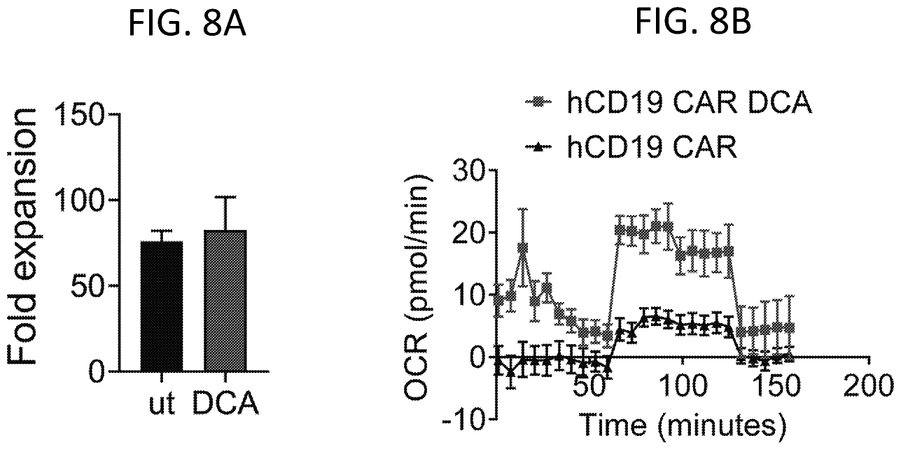

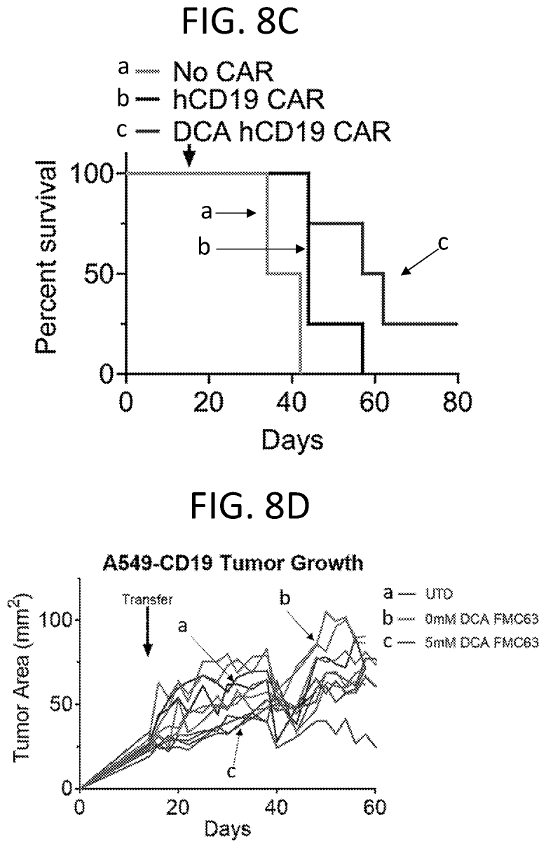

FIG. 8A shows the least 10-fold enhancement in respiratory capacity achieved for mice with T cells expanded in 5 mM DCA. OCR was evaluated at serial time points and it was found that DCA-treated human anti-CD19 CAR-T cells showed a superior metabolic and therapeutic phenotype at 5 mM DCA relative to hCD19 CAR (FIG. 8B). Further, FIG. 8C shows that this observation is associated with enhanced survival. Strikingly, a substantial proportion of the mice survived long term and produced memory to the tumor.

Controls were prepared and human anti-CD19 CAR-T cells were cultured as described above. Metabolic flux rate experiments measuring OCR were also performed as described above.

As shown in FIG. 8D, A549-CD19 cancers in mice were reduced to a greater extent when T cells exposed to DCA are administered (as compared to administration of untreated (UTD) or T cells without exposure to DCA). For example, at about 40-45 days following administration of the treated T cells, tumor volume reduced by about 30-40% on average when DCA treated T cells were used. In addition, there was a significant percentage of complete regressions (12% DCA vs 0% UTD).

### Example 8

### DCA can Metabolically Enhance a Solid-Tumor Targeted Car

This example demonstrates that EGFR BBz CAR-T cells expanded in the presence of PDHK1 inhibitor (DCA) show improved respiratory capacity and efficacy in a solid tumor mouse model.

Immunodeficient mice with a solid tumor (unmodified A549) were treated with adoptively transferred EGFR BBz CAR T cells that were expanded either in the presence or absence of DCA.

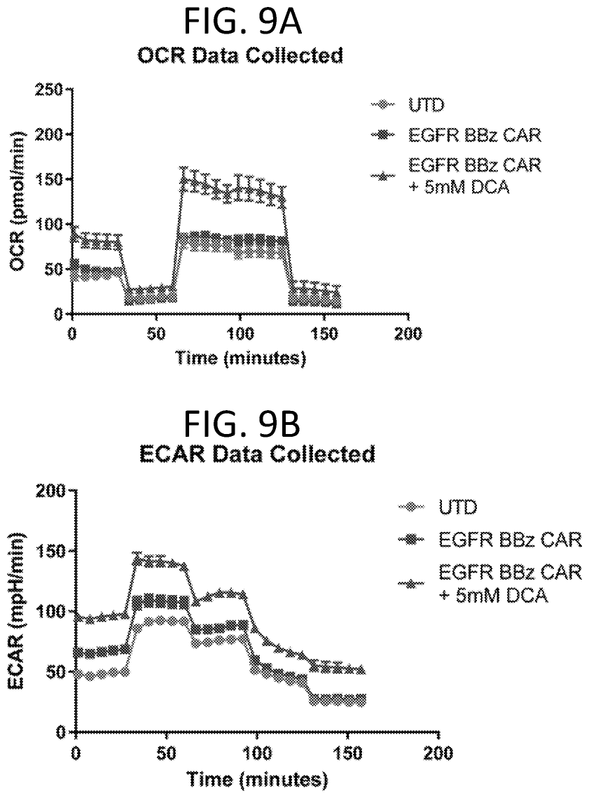

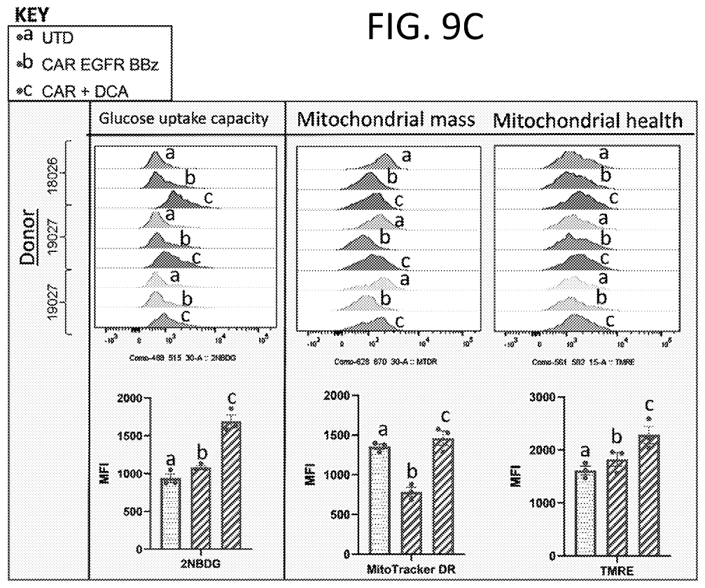

FIGS. 9A-9C depict metabolic flux rate experiments and flow cytometry measurements, showing that DCA can metabolically enhance a solid-tumor targeted CAR. FIG. 8A shows that DCA-treated EGFR BBz CAR-T cells display a superior metabolic phenotype at 5 mM DCA and 10 mM DCA relative to UTD control as measured by OCR analysis. FIG. 9B shows that DCA-treated EGFR BBz CAR-T cells display a superior metabolic phenotype at 5 mM DCA and 10 mM DCA relative to UTD control as measured by ECAR analysis.

FIG. 9C demonstrates that the observations in FIGS. 9A-9B are associated with a superior effector phenotype for T cells infiltrating the tumor, including a reduction of mitochondrial mass, improved mitochondrial function, and enhanced glucose uptake. To assay the metabolic capacity of tumor-infiltrating T cells, mitochondrial function and mass are measured by flow cytometry using MitoTracker® Deep Red FM (a membrane permeable, carbocyanine-based dye for mitochondria) and competency for glucose uptake using fluorescent 2-NBD-glucose (2NBDG) in T cells infiltrating implantable tumors (FIG. 8C). MitoTracker® Deep Red is highly resistant to collapse of membrane potential, especially compared to tetra-methylrhodamine ester (TMRE), a well-known membrane-potential sensitive dye. Mice were inoculated with A549, and at day 10, lymph node and tumor preparations were pulsed with 2-NBDG and stained for flow cytometric analysis. T cells in the lymph nodes effectively took up glucose and had relatively high MitoTracker® FM staining.

The UTD control was prepared as described above. Metabolic flux rate experiments and T cell preparation were performed as described above.

### Example 9

### DCA Treatment Improves Functional Memory and T-Cell Persistence

This example demonstrates that DCA treatment improves T cell functional memory, persistence, and viability post-infusion, in a solid tumor mouse model.

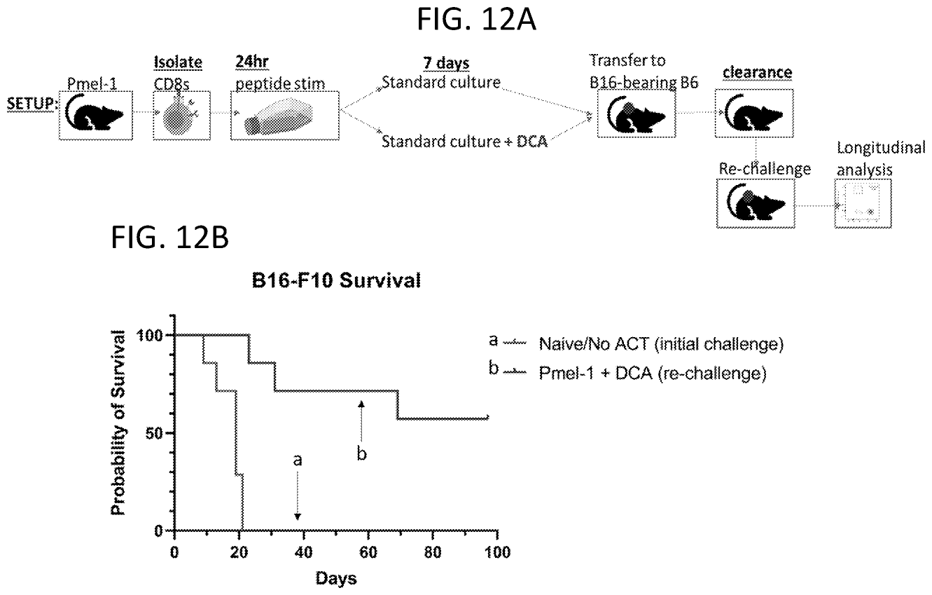

Pmel T cells were isolated and expanded as described in Example 1 in the presence or absence of DCA. After 7 days of culture, T cells were transferred to B16 tumor-bearing mice. After tumor clearance, mice were rechallenged with B16 tumors (see, FIG. 12A). The percent survival of the mice was measured at intervals extending out to 100 days (FIG. 12B). Rechallenged mice that received Pmel-1+DCA treatment showed improved survival. For example, about 70% of the treated mice were alive at 40-70 days, and about 60% of the mice were alive at 80-100 days, as compared to none of the untreated mice. Thus, mice cured with DCA expanded Pmel T cells, and subsequently rechallenged with B16, had slower tumor growth and continued survival. This demonstrates that DCA expanded T cells provide robust immunologic memory to the tumor.

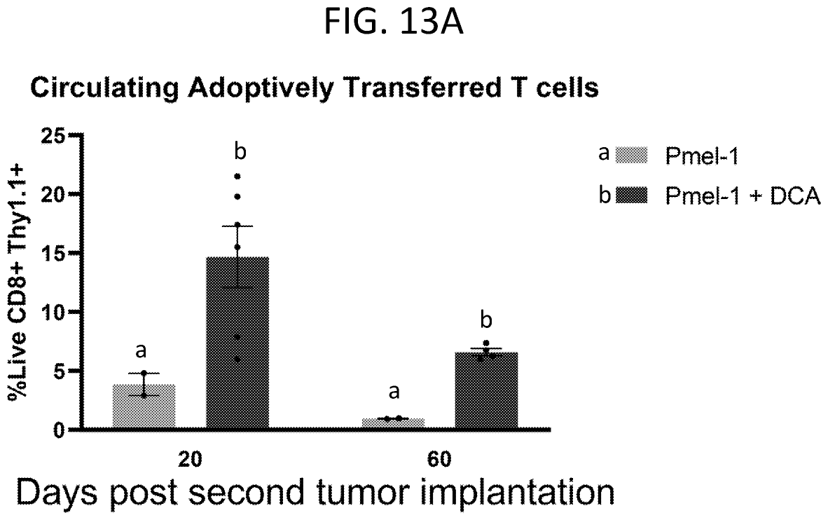

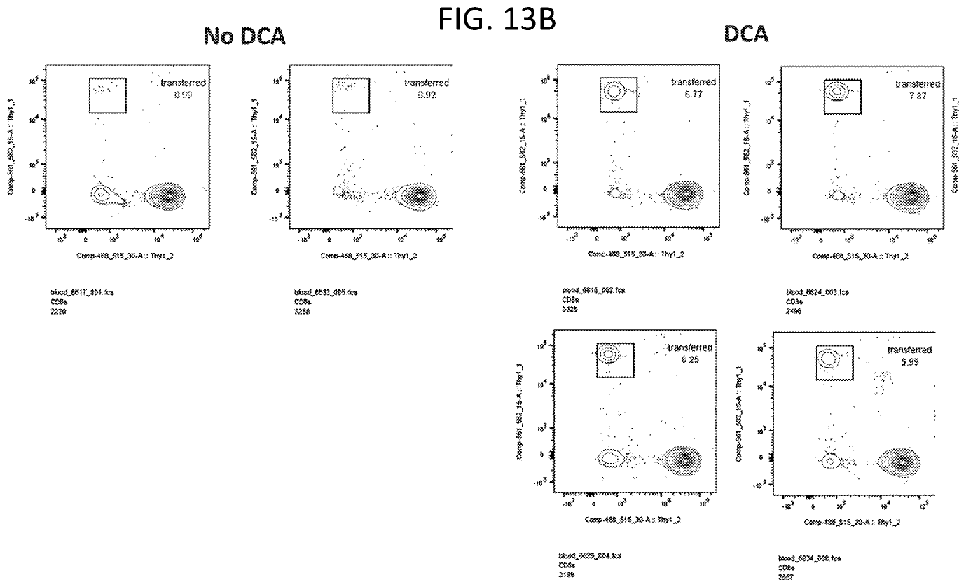

The persistence of the therapeutic T cells was measured by congenic marker staining (Thy1.1). In mice cured with DCA expanded T cells (DCA), the therapeutic T cells persisted within the blood at much higher levels (5-15% of all CD8 T cells) compared to control Pmel cells, well after tumors were cleared. Circulating adoptively transferred T cells were also significantly higher in mice treated with Pmel+DCA (FIGS. 13A and 13B). For example, about 10-18% (such as about 15%) of circulating adoptively transferred T cells treated with DCA were alive 20 days after the second tumor implantation, as compared to about 3-5% of circulating adoptively transferred T cells not treated with DCA. Along the same trend, about 5-8% (such as 6-7%) of circulating adoptively transferred T cells treated with DCA were alive 60 days after the second tumor implantation, as compared to less than 1% of circulating adoptively transferred T cells not treated with DCA. The results demonstrate that DCA treatment improves functional T cell memory and persistence in vivo.

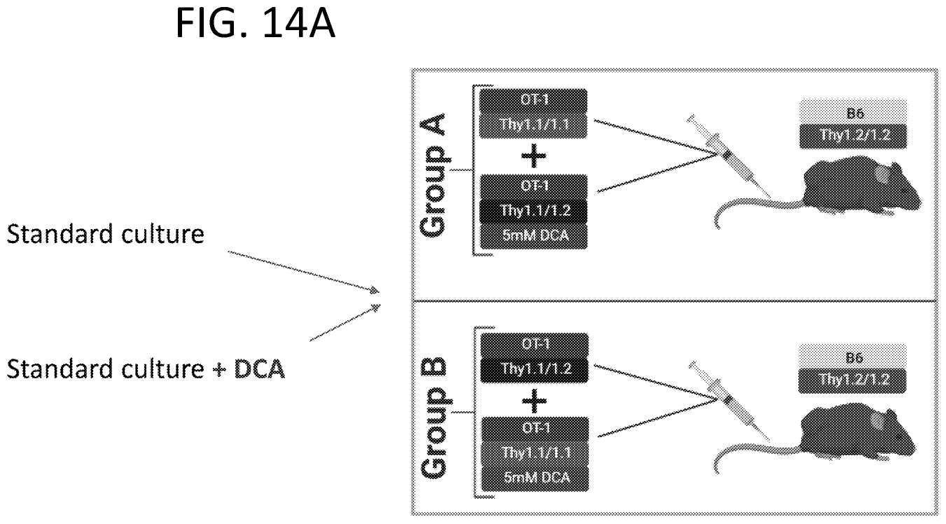

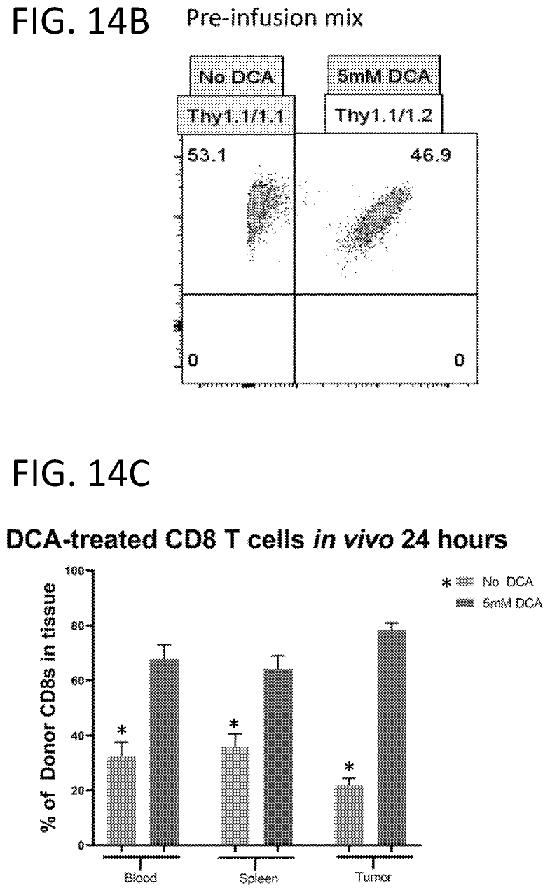

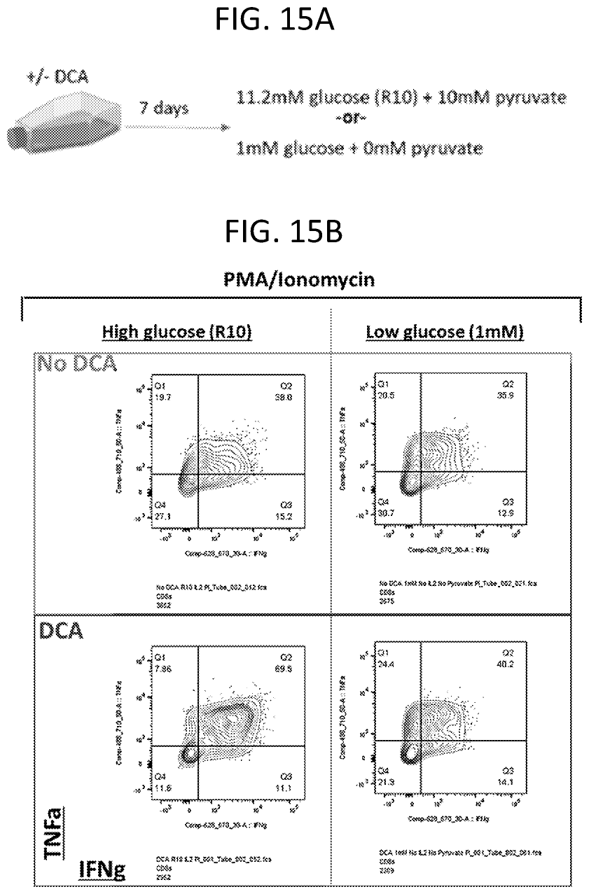

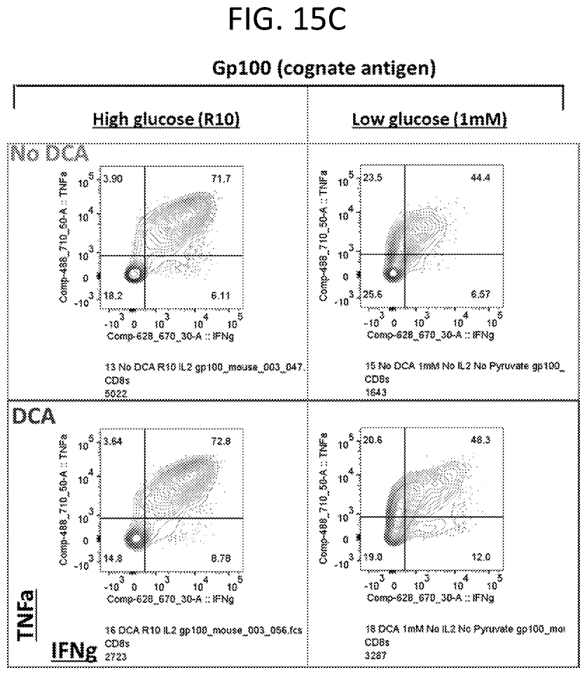

FIG. 14A provides an overview of the method used to demonstrate that DCA expanded T cells show immediate increase in viability post-infusion. Congenically marked (Thy1.1 vs Thy1.1/Thy1.2) T cells expanded with or without 5 mM DCA were mixed 50:50 and transferred to B16 tumor-bearing mice. DCA expanded T cells had an immediate numerical advantage in blood, lymphoid organs, and within the tumor (see, FIGS. 14B-14C). For example, about 60-80% of the donor CD8 cells in the tissue tested were DCA-treated CD8 T cells, while only about 20-40% of the donor CD8 cells in the tissue tested were untreated CD8 T cells.

In view of the many possible embodiments to which the principles of the disclosure may be applied, it should be recognized that the illustrated embodiments are only examples of the invention and should not be taken as limiting the scope of the invention. Rather, the scope of the invention is defined by the following claims. We therefore claim as our invention all that comes within the scope and spirit of these claims.

1. A method of treating cancer, infectious disease, autoimmune disease, inflammatory disease, or immunodeficiency in a subject, comprising administering to the subject a therapeutically effective amount of immune effector cells,
wherein the immune effector cells are prepared by:
(a) activating a population of immune effector cells, thereby producing activated immune effector cells;
(b) transducing the activated immune effector cells with a vector comprising a polynucleotide encoding an engineered antigen receptor, thereby producing transduced immune effector cells; and
(c) expanding the transduced immune effector cells in the presence of a metabolic enhancer selected from the group consisting of a pyruvate dehydrogenase kinase 1 (PDHK1) inhibitor, a pyruvate dehydrogenase phosphatase (PDP) activator, a peroxisome proliferator-activated receptor gamma coactivator 1-alpha (PGC1α) polypeptide or a variant thereof, and a PGC1α agonist.

2. The method of claim 1, wherein the method further comprises isolating or obtaining peripheral blood mononuclear cells (PBMCs) as a source of the population of immune effector cells.

3. The method of claim 1, wherein the PDHK1 inhibitor comprises a peptide, a PDHK1 antibody or antibody fragment, a small molecule, a PDHK1 inhibitory RNA (RNAi) molecule, or a PDHK1 aptamer;
the PDP activator comprises a PDP protein, peptide, an enzyme, a PDP antibody, a PDP antibody fragment, PDP coding sequence, or a small molecule; or
the PGC1α polypeptide or variant thereof is:
(i) a full length PGC1α polypeptide, a PGC1α polypeptide comprising one or more amino acid insertions, deletions, or substitutions, or a biologically active PGC1α polypeptide fragment; or
(2) a biologically active PGC1α polypeptide fragment comprising amino acids 1-270 of PGC1α, amino acids 1-290 of PGC1α, no more than about the first (N-terminal) 270 amino acids of PGC1α or no more than about the first (N-terminal) 290 amino acids of PGC1α.

4. The method of claim 1, wherein the PDHK1 inhibitor comprises dichloroacetic acid (DCA) or AZD7545.

5. The method of claim 1, wherein the PDHK1 inhibitor comprises dichloroacetic acid (DCA).

6. The method of claim 1, wherein the PDP activator comprises insulin, PEP, or AMP.

7. The method of claim 1, wherein the metabolic enhancer comprises DCA and the method further comprises:
(i) electroporating the immune effector cells with an mRNA encoding a biologically active PGC1α polypeptide fragment selected from the group consisting of amino acids 1-270 of PGC1α, amino acids 1-290 of PGC1α, no more than about the first (N-terminal) 270 amino acids of PGC1α, or no more than about the first (N-terminal) 290 amino acids of PGC1α; or
(ii) transducing the immune effector cells with a vector comprising a polynucleotide encoding an engineered antigen receptor and a polynucleotide encoding a biologically active PGC1α polypeptide fragment selected from the group consisting of amino acids 1-270 of PGC1α, amino acids 1-290 of PGC1α, no more than about the first (N-terminal) 270 amino acids of PGC1α, or no more than about the first (N-terminal) 290 amino acids of PGC1α.

8. The method of claim 1, wherein the immune effector cells comprise or consist of T cells.

9. The method of claim 1, wherein the engineered antigen receptor is an engineered T cell receptor (TCR), a chimeric antigen receptor (CAR), or a dimerizing agent regulated immunoreceptor complex (DARIC).

10. The method of claim 1, wherein activating the population of immune effector cells comprises stimulating the population of immune effector cells with (i) interleukin 2 (IL-2); (ii) an anti-CD3 antibody or antigen binding fragment thereof; and (iii) an anti-CD28 antibody or antigen binding fragment thereof.

11. The method of claim 1, wherein expanding the transduced immune effector cells comprises culturing the transduced immune effector cells in the presence of the one or more metabolic enhancers for at least 6 days.

12. The method of claim 1, comprising treating cancer in the subject.

13. The method of claim 12, wherein the cancer is Wilms' tumor, Ewing sarcoma, a neuroendocrine tumor, a glioblastoma, a neuroblastoma, a melanoma, skin cancer, breast cancer, colon cancer, rectal cancer, prostate cancer, liver cancer, renal cancer, pancreatic cancer, lung cancer, biliary cancer, cervical cancer, endometrial cancer, esophageal cancer, gastric cancer, head and neck cancer, medullary thyroid carcinoma, ovarian cancer, glioma, bone cancer, lymphoma, leukemia, myeloma, acute lymphoblastic leukemia, acute myelogenous leukemia, chronic lymphocytic leukemia, chronic myelogenous leukemia, Hodgkin's lymphoma, non-Hodgkin's lymphoma, or urinary bladder cancer.

14. The method of claim 12, wherein the method increases survival of the subject by at least 20% as compared to administration of immune effector cells expanded in the absence of the metabolic enhancer.

15. The method of claim 12, wherein the method increases survival of the subject by at least 50% as compared to administration of immune effector cells expanded in the absence of the metabolic enhancer.

16. The method of claim 11, wherein expanding in the presence of the metabolic enhancer increases in vivo persistence of the immune effector cells as compared to administration of immune effector cells expanded in the absence of the metabolic enhancer.

17. The method of claim 1, wherein the subject is a mammal.

18. The method of claim 17, wherein the mammal is a human.


SEQUENCE LISTING (24 sequences)

SEQ ID NO: 1 - DNA, 2672 bp, Homo sapiens
  source 1..2672: mol_type = unassigned DNA
gaaacctggcgtactggctgtggcttctctagcgggactcggcatgaggctggcgcggctgcttcgcggagccgccttggccggcccgggcccggggctgcgcgccgccggcttcagccgcagcttcagctcggactcgggctccagcccggcgtccgagcgcggcgttccgggccaggtggacttctacgcgcgcttctcgccgtccccgctctccatgaagcagttcctggacttcggatcagtgaatgcttgtgaaaagacctcatttatgtttctgcggcaagagttgcctgtcagactggcaaatataatgaaagaaataagtctccttccagataatcttctcaggacaccatccgttcaattggtacaaagctggtatatccagagtcttcaggagcttcttgattttaaggacaaaagtgctgaggatgctaaagctatttatgactttacagatactgtgatacggatcagaaaccgacacaatgatgtcattcccacaatggcccagggtgtgattgaatacaaggagagctttggggtggatcctgtcaccagccagaatgttcagtactttttggatcgattctacatgagtcgcatttcaattagaatgttactcaatcagcactctttattgtttggtggaaaaggcaaaggaagtccatctcatcgaaaacacattggaagcataaatccaaactgcaatgtacttgaagttattaaagatggctatgaaaatgctaggcgtctgtgtgatttgtattatattaactctcccgaactagaacttgaagaactaaatgcaaaatcaccaggacagccaatacaagtggtttatgtaccatcccatctctatcacatggtgtttgaacttttcaagaatgcaatgagagccactatggaacaccatgccaacagaggtgtttacccccctattcaagttcatgtcacgctgggtaatgaggatttgactgtgaagatgagtgaccgaggaggtggcgttcctttgaggaaaattgacagacttttcaactacatgtattcaactgcaccaagacctcgtgttgagacctcccgcgcagtgcctctggctggttttggttatggattgcccatatcacgtctttacgcacaatacttccaaggagacctgaagctgtattccctagagggttacgggacagatgcagttatctacattaaggctctgtcaacagactcaatagaaagactcccagtgtataacaaagctgcctggaagcattacaacaccaaccacgaggctgatgactggtgcgtccccagcagagaacccaaagacatgacgacgttccgcagtgcctagacacacttgggacatcggaaaatccaaatgtggcttttgtattaaatttggaagtgtggcccagagttgctcagaattggagcagagcctgagacgtatctgcagatcctgtcatcagctggcaagtccaggagactgtgtcatttagagactgtgttgttagttatccctcaacatcttctaaggtggcaggaaataatattggaaataacattttaaagtaaaaattttaaagtttaaagaagagttttgccacttaaacaggggagctttgtctggaaaatacactgagttgaaacacttcatccttggaaggattatataagatgaacagttgtgataaatgtgtagattagagggatgtgaatgggcagttagtccagtgccctcatttaagaggccaagatcctgattcagaggaggcatcctttgcccagagctgcttagctaatctgaccaaatgttgggaaaaatgtctcacctaacccactattccttaattatggattttgtgaaaaacaatagaacatgttaatgagtaatttatattagttcgatgtattacaattttttagctttaaattacagttttcttataatgttgaaatgttttagaatcctttgaatctaagtatttgtttcctaaatgaaacatttgtacaacatttgatgtttttacttatgaaatattctcctcccccaagaaaatttaaactttttctctctatttaaaagctaagaaatgttttaaaggaaaaatgaaattatcttcctttagcttatttttaaggtaaaacagctttttactctgttattgtggtaatggacagaatattacatacaaaaatattctgggagagctttttcctagttggttttaaatcattgtgccacctgaaaggtttttagattttataggagctaatttgtccaccagcattaatgtaacacagtgtagttatgaaaatatattgaaggacaggaagtggacacgaagtgatttttgtaacctgagcagttaatgaatgtgccaacattttctaggaagggacagcaagaatattctgctctgtagttaaaatactggctggcttttgatgtcttcatgcttaattgtgatcactttcttgcactgtgatgtttttacgtgaatatgttgaagtagaagtctaccatattattttataaaatgttttctgtatggcaataaactgaaaacatggatcaacccttcttttgaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 2 - protein, 436 aa, Homo sapiens
MRLARLLRGAALAGPGPGLRAAGFSRSFSSDSGSSPASERGVPGQVDFYARFSPSPLSMKQFLDFGSVNACEKTSFMFLRQELPVRLANIMKEISLLPDNLLRTPSVQLVQSWYIQSLQELLDFKDKSAEDAKAIYDFTDTVIRIRNRHNDVIPTMAQGVIEYKESFGVDPVTSQNVQYFLDRFYMSRISIRMLLNQHSLLFGGKGKGSPSHRKHIGSINPNCNVLEVIKDGYENARRLCDLYYINSPELELEELNAKSPGQPIQVVYVPSHLYHMVFELFKNAMRATMEHHANRGVYPPIQVHVTLGNEDLTVKMSDRGGGVPLRKIDRLFNYMYSTAPRPRVETSRAVPLAGFGYGLPISRLYAQYFQGDLKLYSLEGYGTDAVIYIKALSTDSIERLPVYNKAAWKHYNTNHEADDWCVPSREPKDMTTFRSA

SEQ ID NO: 3 - DNA, 9146 bp, Homo sapiens
  source 1..9146: mol_type = unassigned DNA
ggcggccccgcacggtaggggagcagagtgggcaggccgggggtgagggctcgcgctccgggagctgcacggggctgcgtggaaagagcgccgagcggtggcgtcgttgtcgccccctcctcgtcgggaagaatcgtttggtctcctgccgtgcccggttcgtattccctactccctgccacgagccgccccgtccgggatcctccacccgtccaaagttgtgagggggcgccgggcgtgctcgcggatcggcggccgcgggcgtgcggagggctggacgagccctggagcgccaggtgcgcgccgggcgtccgggctgtggggcgggcgtggcggcgctgggggccgtcggggggctgggggcgggggcgggggcgccgtgcagccctagcgtcttccggcccctgcgctccagctgtcagcacgaagggcgccgcggggccggcggcagagtgagctccagggaggctgtagggctgcggcactgcctgagggtctccctgacgcaccccgggtcagggctggccccgctggcagcctgacaccggccggcccctgttcgtctggactgaaacgggcacccctggggcggggccgggccgggcagggcggggcgggcagcgggcggggctgcagtccatgggcttcgcgccccggctggatggaggggctggccctcggggcgcacccctgctcgcgcctgggccccgctgccttgggctgtggctttgcctccccggcggggtgggttggtttggttggcttccttgttctctccacccctttatccctcctccatcctcttcccccccacctcccagcccattcaccgggctggagcgagacctcgcccctcctccgctcccgcctccccgccgccgcctcctctaatgagcatctctgccttgctttcaatgggccggtgctgctgtcgctgctgctgcccgcgcgggttgtggatgttgtcggctccgtgttgtgatgacaggagaatgtgtgtgtgtcccgggcccagacgaattggtaggtattcacttaactacctgcgttgggtacccaggaaggatggtgacagatttttgtgtatggatggttttagatgaatcagcgtggtattaagggatgagatagattggaaaaagggacaaacgcgtcttggattgagatagctttgatgtctttgtgagtgcagtttttcaggtagcgacaaaggaaactaatcaaaaaaatactggcgtgtggtgcttcgtatctttcttcatttatgtaaaaagatagcaggagctgtgaaatggctaggcatttccagaaaaatgttgaaaaatcgcggatggtctttatgtagagtgtttaatgcaatcggattacataaactggggttccgacacattaaatgattagcatagcttttctttttcatttacatcagaagctttgcaaagctaaagggctctggagtcggtgccttaatagcatttgctaattaatttggtccgtgggtctattgatggagtggaaaaaatgtcttggcacaggctctgctaagcacatcagtaagaatggacggctctctttcctgtttctggttgcagtgcatctttctttattagtcattggcaaaatcagcccaatctgcctgggatgaagatgctacatacaacgtggctgagtctagatcatggaataggaaattttccgtgaaaattgtgatcaagaataacttgcaaatttgacactgctctgttttaccaacagttgtacctggatgctgttcttattgcctgatcaagagccttctacactaatttaacaagcttgtcagcaaaagttatgatgtagagtgttttcttgttgttgaatcagtggataatctgaaggatattaagagtttttcattgcttacttatttatgaaagcatttgaaagtactttgactcccaagggtatctgatctgatggagagaatgtatcacttgtaccgatgcggatagaacgctcattaattacagcacagttttcataaattccaacttccctaggaattttgtcttttgaaggcatgttggaagttggggcattaggatataatctcagtccatgtactaagcctctgcataactgaaagatagagtcaataaaattcactctctcattttttgtttttaatctcaagaatttttgccttgaaggaaaaatgtagtggagaatttgatgtttgaacgtaagtattgttaaaagcacctgctgtgtcaatcattgctttttgtcactagatttcattcattttgcagtcaattacaaaagtagaaaagatgtcataccaagccgggtgcagtggctcatgcctgtaatcccagcactttgggaggctgaggcgggtggatggcctgaggtcaggagttcgagaccaacctgaccaacatggtgaaaccccatctctactgaaaatacagaattagccgggcaggagaatcgcttgaaccagggaggcggaggttgcgatgagctgagatcacgccattgcacttcagcctgggcaacaagagcaaaattcctccccccgctgccctccctccccccccccggcaaaaaaatgtcgaagcaaaggaatttaaggaaagtgcaggtttccaggacttgttattccttaattaaacaatcaaaccctgttatgcatctatcaaccctccctcccccccacacaaatagtattttgagtcagtttacgttccatacaggcacattttcattgtttttttctgtaagattcttaagttttaaagaaagagcaaatgcagtcataaaattatgaagaaaaattcattaagattacattagtttacatttttaatgatagaaaacctttattcaaagcacaaaatgagttgatatgctttctgcacgcaaacctaaaaagagatatggtttaagtatggaagccactgtcatgtggcccaacatttgagcacatcttctggctcagtcatgtacacttaacctctggatgactttcaggtattcagtttgaatttcttttatttaaatggttactcctgatggattcccgtttttctggaatttctgtttttcattagtgcatttcagaatgcttttttagttacggattcttatttccaaaattttagccattactttctattgcctgatcttgtcttcattttcgtaaacaaatcccttcttagatagtgcagttaaaatgatggtactttctactacttgcagaagagaatctgtgtggctggcttatgcagtgcttattacagaaacaaacaagaatcagtgattattcagtgtttagttttctttgcttccctctaccccaatttattactttttctcaaattattcttcaaaaaaggtacatgaaaataatatatggctgacacatgctatgatatttgtttagccgtgggaaggaatagttgctattaactcagatggttttattttctgtgttcctttttgtttattgcatttgtttatttaaagtatttccaaagtgtttctttatagtacaaatgactattttgaagcagtatctcaagaattcagggaaaatatatgttattgaggaagaagagggattttttttcttgcccaaattatctagattaatgatatattcattaaatttcccaaataaatgcccagaaattattttcagcgcacatagtgagtgataggattttttttaagtttgtaattatattgctgtaccttttcaatccaaaccttagactattatttgactatactgcttccctggagcaagtaaataccttttttttttttttttaatgtgagagaaaacatttttggtcttttaaaatttatttttaatttacaattgacacataatattatatatttatggggtacaatgtgatgtttcagtgcatgtatatattggtagacattgtgtaatgatcaaatcagagtgattaccatatccaagaaaacatttttctaggggttaattctactttggatccatagtagatgaaaatacttctatgggttttccttatagctgctaagcagggaacattgttagtaggtaaattaagttctaggactgaaaccatttatgttactaacaaagctttagagctactgcaatacttaaactttttttttttttttaatcatgaagggcacggtagaattcaacagtgaataaatgaaacctgaattttatttaaaaacctcacttccataagcataaaggcatgaatcctagaagagccttagatagcatctgctctgttgatgttcctttacaggttagaagagtgagactcaaagatgtgagctgacttttccaagattatttgggagttggtggcagacctatgcccgttagcctggcaaattgccacagtggactagagaagcaagttttgcaagaccatctactggggtacaggagagaaatactggaatttctttttttatttaattatgtagaaatttttctatatatgttacaatgcatattactgtagtcatatatgtatatgatgtacacgtaattcataaatacacatatgtttggctgcctcttaacatttttggtcatagccatgtgtaatcatgccaaaaattgaccacaggtctatgtaaagggtcttcataaatggaagaagtccctgtgtaaaatgggagatttcaaggcattttttaagaatagggagggagaaggatgattggtcaggtagacggctactgcataatgttatataaccttcatctcctcagctttccccagccaaatctgttgtacattctgcacagtttgtactgcacaaaggacgacaaagaaaagagaaaattgaagtttgagtataggagccaggagttcagtcgtggttccacgaatgattgggagttgtgcctgccagaatataccctgtagaaatagtacatgatgtgtgtataaaatgtagtaccttctagcaaaggcttaacatgtagattgacttgcttaatatttgttataaaggcaaagctagattaagtatcctgaaatgtaactctttcctttgtgtacatctgtctaatttgctgtctaggaatcccagtcagaagttccagcctgccactgttctctgatgccatgccagcaccaactcaactgttttttcctctcatccgtaactgtgaactgagcaggatctatggcactgcatgttactgccaccacaaacatctctgttgttcctcatcgtacattcctcagagtcgactgagatacacacctcatccagcatatgctaccttttgcaggccaaaggagaactggtggcagtacacccaaggaaggagatatgcttccacaccacagaaattttacctcacacctccacaagtcaatagcatccttaaagctaatgaatacagtttcaaagtgccagaatttgacggcaaaaatgtcagttctatccttggatttgacagcaatcagctgcctgcaaatgcacccattgaggaccggagaagtgcagcaacctgcttgcagaccagagggatgcttttgggggtttttgatggccatgcaggttgtgcttgttcccaggcagtcagtgaaagactcttttattatattgctgtctctttgttaccccatgagactttgctagagattgaaaatgcagtggagagcggccgggcactgctacccattctccagtggcacaagcaccccaatgattactttagtaaggaggcatccaaattgtactttaacagcttgaggacttactggcaagagcttatagacctcaacactggtgagtcgactgatattgatgttaaggaggctctaattaatgccttcaagaggcttgataatgacatctccttggaggcgcaagttggtgatcctaattcttttctcaactacctggtgcttcgagtggcattttctggagccactgcttgtgtggcccatgtggatggtgttgaccttcatgtggccaatactggcgatagcagagccatgctgggtgtgcaggaagaggacggctcatggtcagcagtcacgctgtctaatgaccacaatgctcaaaatgaaagagaactagaacggctgaaattggaacatccaaagagtgaggccaagagtgtcgtgaaacaggatcggctgcttggcttgctgatgccatttagggcatttggagatgtaaagttcaaatggagcattgaccttcaaaagagagtgatagaatctggcccagaccagttgaatgacaatgaatataccaagtttattcctcctaattatcacacacctccttatctcactgctgagccagaggtaacttaccaccgattaaggccacaggataagtttctggtgttggctactgatgggttgtgggagactatgcataggcaggatgtggttaggattgtgggtgagtacctaactggcatgcatcaccaacagccaatagctgttggtggctacaaggtgactctgggacagatgcatggccttttaacagaaaggagaaccaaaatgtcctcggtatttgaggatcagaacgcagcaacccatctcattcgccacgctgtgggcaacaacgagtttgggactgttgatcatgagcgcctctctaaaatgcttagtcttcctgaagagcttgctcgaatgtacagagatgacattacaatcattgtagttcagttcaattctcatgttgtaggggcgtatcaaaaccaagaatagtgagtggctctttcactggcaattctcaaatgatatacatttaaagggcagattttttaaaaagatactactataataaacatttccagttggtcattctaagcatttacccttttgatactctagctagtcaggtactccaaattgactttgcagcagggtggcagggtcaggagagtctggtcctgcctagctcagatttcatggcacctgcacttgaagcaagtcacttctttatcacaggtgtcttgaaacattagcttcttttaccaacctgagaaaattaggatgacctggcaaataagatcttgaataggccaaaagcaagtatcttgctgtgtgtagtctcttggttaaagtgaagaaacagtactgttcacacctttcttcactgagattccagtgtacatgagaacatatatttattgcatgattttctagatacacagtctatgcattattcatatacatttattttagcctaaagtggttttcaaatccagttcttcaagccataaatgaccaagatccaagcaatctgaatttgtttttgtgattatttgactggaatgcttcttaagtggaataactatactccgttatccacccgatttcctaatgtaattgaaagattttctattttgccacacacttggagacaataagggtttttagttttatctactcttctattgaagttaaagaaagaaaaaaagatttttttatttgtattaatgaaaagctttagtttaaaataaggagatccagaataaaaagaagagactgatctcttcaattattgtcatctgtagccaccagcacatcactcttatgtaatccccaaaggcttggcatgccgtaagtgtgtggtgggtagactgctgccggggaatcgtacttcttatttagtaatgataagacttttcattatttttggaattttaaagatgacataaataagtttaaatatcaatttggggagtaaggtttaatattgccatcgggtattgagacaggaggaagtttctgtttttctccatttagacataggtcaattaaaatatttgggtttaaaatgactaaatgctttaaacatattgtagcttaagatatatgtgttaagatatatacatgagaaactttaaaaggtaactactgtgcatgcctgatgcttaatagaatacttagtggcatcaaatgtttgcagcagtctccataattatattcagtcccttctaatactgtatcaatgtaaatgaaataaatatattcaaattggctttttgatatgcatcaagtggcattttgttcctgtgtttaatagtgatctgtatacagctgtgcacatattgtcatcacttattctagcatcactgttaaggctgtgattatgtttgatattcacctggattttaatacaagccaatatcagcttcccattgtgtaataacttgggtgtttaggagtcttttcacattttttggggatatgaactagatgttcaagaactccttctggactgtggatactgaatcagtgtactattggctgcagaatttgtttcaattgaaaatagactcaggaagattgctgctcagaatatcatataatgtttattttttgaggtgtttttgtttttatttgtgtgtttttttttttttaagtcagcttggaacttttttcctgggtagtatttgggagagggaaaggctgtactatatatttatttctaaatgttttgactgggcatttttcttttaatgaaatatgtggactgctctagcaaaccctattttcagctactatttgaatattcttgaacaccaccactgaagagtttcatatacaccaaataatgtctcatctctatagtacagggaatataaaattggtttcctgtggtcatgatcaagatagtagtattattacacaagaaacttggtctgcagtctggaagcttgtctgctctatagaaatgaaaatgcagcatgaagttgacattgtggaaatgaaagtaattgggtattagaaatctgaaagtactgtcatctaaaagcaattgtgattttattgtaattggttgtcactgttgtacggtgtctagaattaaagaatacatgtaaactttcatggtatttagcctttcttaaatttttttaaaatttaaactttctaacctatgtattcaacttctgtatttatatttaatcagtggttcatgttatataatacacccttaactagttaaatggaatgttggtatggtacagagtaccatattgctaagaaaactgtcttataaaagatgtatatgtgtgaagacatgaaagtttaatgtacagaatggttggagaaatgcctatggtgaattaaagcttcatatctgctttctgaa

SEQ ID NO: 4 - protein, 537 aa, Homo sapiens
MPAPTQLFFPLIRNCELSRIYGTACYCHHKHLCCSSSYIPQSRLRYTPHPAYATFCRPKENWWQYTQGRRYASTPQKFYLTPPQVNSILKANEYSFKVPEFDGKNVSSILGFDSNQLPANAPIEDRRSAATCLQTRGMLLGVFDGHAGCACSQAVSERLFYYIAVSLLPHETLLEIENAVESGRALLPILQWHKHPNDYFSKEASKLYFNSLRTYWQELIDLNTGESTDIDVKEALINAFKRLDNDISLEAQVGDPNSFLNYLVLRVAFSGATACVAHVDGVDLHVANTGDSRAMLGVQEEDGSWSAVTLSNDHNAQNERELERLKLEHPKSEAKSVVKQDRLLGLLMPFRAFGDVKFKWSIDLQKRVIESGPDQLNDNEYTKFIPPNYHTPPYLTAEPEVTYHRLRPQDKFLVLATDGLWETMHRQDVVRIVGEYLTGMHHQQPIAVGGYKVTLGQMHGLLTERRTKMSSVFEDQNAATHLIRHAVGNNEFGTVDHERLSKMLSLPEELARMYRDDITIIVVQFNSHVVGAYQNQE

SEQ ID NO: 5 - skipped

SEQ ID NO: 6 - protein, 7 aa, synthetic construct
  REGION 1..7: note = Exemplary tobacco etch virus (TEV) protease cleavage sites
ENLYFQG

SEQ ID NO: 7 - protein, 7 aa, synthetic construct
  REGION 1..7: note = Exemplary tobacco etch virus (TEV) protease cleavage site
ENLYFQS

SEQ ID NO: 8 - protein, 5 aa, synthetic construct
  REGION 1..5: note = Exemplary linker
DGGGS

SEQ ID NO: 9 - protein, 5 aa, synthetic construct
  REGION 1..5: note = Exemplary linker
TGEKP

SEQ ID NO: 10 - protein, 4 aa, synthetic construct
  REGION 1..4: note = Exemplary linker
GGRR

SEQ ID NO: 11 - protein, 5 aa, synthetic construct
  REGION 1..5: note = Exemplary linker
  REPEAT 1..5: note = Sequence repeated 1, 2, 3, 4 or 5 times
GGGGS

SEQ ID NO: 12 - protein, 14 aa, synthetic construct
  REGION 1..14: note = Exemplary linker
EGKSSGSGSESKVD

SEQ ID NO: 13 - protein, 18 aa, synthetic construct
  REGION 1..18: note = Exemplary linker
KESGSVSSEQLAQFRSLD

SEQ ID NO: 14 - protein, 8 aa, synthetic construct
  REGION 1..8: note = Exemplary linker
GGRRGGGS

SEQ ID NO: 15 - protein, 9 aa, synthetic construct
  REGION 1..9: note = Exemplary linker
LRQRDGERP

SEQ ID NO: 16 - protein, 12 aa, synthetic construct
  REGION 1..12: note = Exemplary linker
LRQKDGGGSERP

SEQ ID NO: 17 - protein, 16 aa, synthetic construct
  REGION 1..16: note = Exemplary linker
LRQKDGGGSGGGSERP

SEQ ID NO: 18 - protein, 798 aa, Homo sapiens
MAWDMCNQDSESVWSDIECAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLTPESPNDPKGSPFENKTIERTLSVELSGTAGLTPPTTPPHKANQDNPFRASPKLKSSCKTVVPPPSKKPRYSESSGTQGNNSTKKGPEQSELYAQLSKSSVLTGGHEERKTKRPSLRLFGDHDYCQSINSKTEILINISQELQDSRQLENKDVSSDWQGQICSSTDSDQCYLRETLEASKQVSPCSTRKQLQDQEIRAELNKHFGHPSQAVFDDEADKTGELRDSDFSNEQFSKLPMFINSGLAMDGLFDDSEDESDKLSYPWDGTQSYSLFNVSPSCSSFNSPCRDSVSPPKSLFSQRPQRMRSRSRSFSRHRSCSRSPYSRSRSRSPGSRSSSRSCYYYESSHYRHRTHRNSPLYVRSRSRSPYSRRPRYDSYEEYQHERLKREEYRREYEKRESERAKQRERQRQKAIEERRVIYVGKIRPDTTRTELRDRFEVFGEIEECTVNLRDDGDSYGFITYRYTCDAFAALENGYTLRRSNETDFELYFCGRKQFFKSNYADLDSNSDDFDPASTKSKYDSLDFDSLLKEAQRSLRR

SEQ ID NO: 19 - protein, 803 aa, Homo sapiens
MDETSPRLEEDWKKVLQREAGWQCAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLTPESPNDPKGSPFENKTIERTLSVELSGTAGLTPPTTPPHKANQDNPFRASPKLKSSCKTVVPPPSKKPRYSESSGTQGNNSTKKGPEQSELYAQLSKSSVLTGGHEERKTKRPSLRLFGDHDYCQSINSKTEILINISQELQDSRQLENKDVSSDWQGQICSSTDSDQCYLRETLEASKQVSPCSTRKQLQDQEIRAELNKHFGHPSQAVFDDEADKTGELRDSDFSNEQFSKLPMFINSGLAMDGLFDDSEDESDKLSYPWDGTQSYSLFNVSPSCSSFNSPCRDSVSPPKSLFSQRPQRMRSRSRSFSRHRSCSRSPYSRSRSRSPGSRSSSRSCYYYESSHYRHRTHRNSPLYVRSRSRSPYSRRPRYDSYEEYQHERLKREEYRREYEKRESERAKQRERQRQKAIEERRVIYVGKIRPDTTRTELRDRFEVFGEIEECTVNLRDDGDSYGFITYRYTCDAFAALENGYTLRRSNETDFELYFCGRKQFFKSNYADLDSNSDDFDPASTKSKYDSLDFDSLLKEAQRSLRR

SEQ ID NO: 20 - DNA, 20 bp, synthetic construct
  misc_feature 1..20: note = Exemplary phosphorothioate antisense oligonucleotides
  source 1..20: mol_type = other DNA
agctggctggtggtcctggc

SEQ ID NO: 21 - DNA, 20 bp, synthetic construct
  misc_feature 1..20: note = Exemplary phosphorothioate antisense oligonucleotides
  source 1..20: mol_type = other DNA
tgaatgatgcccttgccgtg

SEQ ID NO: 22 - DNA, 21 bp, synthetic construct
  misc_feature 1..21: note = Exemplary PDK1 RNAi sequence
  source 1..21: mol_type = other DNA
gctctgtcaacagactcaata

SEQ ID NO: 23 - DNA, 21 bp, synthetic construct
  misc_feature 1..21: note = Exemplary PDK1 RNAi sequence
  source 1..21: mol_type = other DNA
catccgttcaattggtacaaa

SEQ ID NO: 24 - DNA, 21 bp, synthetic construct
  misc_feature 1..21: note = Exemplary PDK1 RNAi sequence
  source 1..21: mol_type = other DNA
cgtgaatatgttgaagtagaa